SEQUENCE LISTING (36 sequences)

SEQ ID NO: 1 - protein, 10 aa, synthetic construct
GYTFTSYNMH

SEQ ID NO: 2 - protein, 17 aa, synthetic construct
AIYPGNGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct [sequence omitted]

SEQ ID NO: 8 - protein, 106 aa, synthetic construct [sequence omitted]

SEQ ID NO: 9 - protein, 5 aa, synthetic construct
NYYIH

SEQ ID NO: 10 - protein, 17 aa, synthetic construct
WIYPGDGNTKYNEKFKG

SEQ ID NO: 11 - protein, 10 aa, synthetic construct
DSYSNYYFDY

SEQ ID NO: 12 - protein, 17 aa, synthetic construct
KSSQSLLNSRTRKNYLA

SEQ ID NO: 13 - protein, 7 aa, synthetic construct
WASTRES

SEQ ID NO: 14 - protein, 8 aa, synthetic construct
TQSFILRT

SEQ ID NO: 15 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 25 aa, synthetic construct [sequence omitted]

SEQ ID NO: 18 - protein, 14 aa, synthetic construct
WVRQAPGKGLEWVG

SEQ ID NO: 19 - protein, 32 aa, synthetic construct
RFTISVDKSKNTLYLQMNSLRAEDTAVYYCAR

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WYQQKPGKAPKPLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGYTFT

SEQ ID NO: 26 - protein, 14 aa, synthetic construct
WVRQAPGQGLEWIG

SEQ ID NO: 27 - protein, 32 aa, synthetic construct
RATLTADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQPPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0256809 A1
(43) Pub. Date: Sep. 3, 2026

(54) METHODS AND COMPOSITIONS FOR TREATING SYSTEMIC LUPUS ERYTHEMATOSUS (SLE) WITH MOSUNETUZUMAB

(71) Applicant: Genentech, Inc., South San Francisco, CA (US)

(72) Inventors: Jay Prakash GARG, Menlo Park, CA (US); Justine Colette MALLER, Half Moon Bay, CA (US); Hardik MODY, Collegeville, PA (US); Theodore Ari OMACHI, San Francisco, CA (US)

(21) Appl. No.: 19/551,018

(22) Filed: Feb. 26, 2026

Related U.S. Application Data
(60) Provisional application No. 63/765,150, filed on Feb. 28, 2025.
(60) Provisional application No. 63/816,829, filed on Jun. 3, 2025.
(60) Provisional application No. 63/944,625, filed on Dec. 19, 2025.

(51) Int. Cl. A61K 31/573; A61K 31/198; A61K 31/454; A61K 31/4709; A61K 31/4745; A61K 31/506

(52) U.S. Cl. CPC A61K 31/573; A61K 31/198; A61K 31/454; A61K 31/4709; A61K 31/4745; A61K 31/506

(57) ABSTRACT

The present invention relates to the treatment of individuals having systemic lupus erythematosus (SLE) using a bispecific antibody that targets CD20 and CD3 (e.g., mosunetuzumab). More specifically, the invention pertains to the treatment of individuals having SLE by subcutaneous administration of mosunetuzumab.

### SEQUENCE LISTING

The instant application contains a Sequence Listing which has been submitted electronically in XML format and is hereby incorporated by reference in its entirety. Said XML copy, created on Feb. 23, 2026, is named 50474-374004_Sequence_Listing_2_23_26.xml and is 33,995 bytes in size.

### FIELD OF THE INVENTION

The present invention relates to the treatment of systemic lupus erythematosus (SLE). More specifically, the invention pertains to treatment of an individual or individuals having SLE by administration of mosunetuzumab, a bispecific antibody that binds to anti-cluster of differentiation 20 (CD20) and anti-cluster of differentiation 3 (CD3). In some instances, mosunetuzumab is administered subcutaneously.

### BACKGROUND

Systemic lupus erythematosus (SLE) is an autoimmune rheumatic disease that occurs primarily in women of childbearing age. It is characterized by multisystem involvement and immunological abnormalities, including dysfunction of B cells and T cells. Much of the tissue damage is thought to occur through autoantibody formation and immune complex deposition, but the underlying cause remains unknown. Disease manifestations can range from mild to severe and potentially life-threatening and may display acute, subacute, and chronic patterns of progression. The protean and heterogeneous nature of SLE makes the diagnosis and management of SLE particularly challenging.

Current standard-of-care therapies for SLE include oral corticosteroids (OCSs), antimalarial therapies (such as hydroxychloroquine, chloroquine, and quinacrine), and conventional immunosuppressants (such as mycophenolate mofetil (MMF), azathioprine, and methotrexate). More recently, belimumab (BENLYSTA), an inhibitor of B-lymphocyte stimulator was approved for the treatment of patients with active, autoantibody-positive SLE who are receiving standard therapy (Burness and McCormack, Drugs. 71(18):2435-2444, 2011). These agents generally have incomplete efficacy for inflammatory manifestations of SLE and their use is frequently limited by toxicities. For example, corticosteroids are effective for many of the manifestations of SLE but have significant short- and long-term adverse effects, including infections, osteoporosis, hyperglycemia, and hyperlipidemia. The unmet medical need in SLE is high.

Despite the available therapies, patients with SLE remain at increased risk for damage accrual, cardiovascular disease, complications of therapy, and death (Cook et al., J. Rheumatol. 27(8):1892-1895, 2000; Gladman et al., J. Rheumatol. 29(2):288-291, 2002; Nossent et al., Lupus. 19(8):949-956, 2010, Lopez et al., Rheumatology (Oxford). 51(3):491-498, 2012). The mortality risk is estimated to be 1.3-5.3 times greater than that of age-matched controls from the general population of the same region (Singh and Yen, Lupus. 27(10):1577-1581, 2018). SLE ranks as one of the leading causes of death in young women (15-24 years old) in the United States (Yen and Singh, Arthritis Rheumatol. 70(8):1251-1255, 2018).

New methods for treating SLE are needed.

### SUMMARY OF THE INVENTION

The present invention relates to methods of treating an individual having systemic lupus erythematosus (SLE) by administration of mosunetuzumab, wherein the treatment comprises subcutaneous administration of mosunetuzumab. In some embodiments, the treatment comprises subcutaneous administration of mosunetuzumab as a monotherapy, e.g., for the treatment of SLE.

In one aspect, the invention features a method of treating an individual having systemic lupus erythematosus (SLE), comprising subcutaneously administering to the individual mosunetuzumab according to a dosing regimen comprising at least a first dosing cycle and a second dosing cycle, wherein: (a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg); and (b) the second dosing cycle comprises a single dose (C2D1) of mosunetuzumab, wherein the C2D1 of mosunetuzumab is about 45 mg (e.g., 45 mg±10%; 45 mg±4.5 mg).

In some embodiments, the first dosing cycle is a 21-day dosing cycle. In some embodiments, the C1D1, C1D2, and C1D3 are administered on or about Days 1, 8, and 15, respectively, of the first dosing cycle. In some embodiments, the second dosing cycle is a 21-day dosing cycle. In some embodiments, the C2D1 is administered on or about Day 1 of the second dosing cycle.

In some embodiments, the method further comprises one or more additional dosing cycles. In some embodiments, the method comprises one to seven (e.g., one, two, three, four, five, six, or seven) additional dosing cycles. In some embodiments, the method comprises seven additional dosing cycles. In some embodiments, each additional dosing cycle is a 21-day dosing cycle. In some embodiments, one or more of the additional dosing cycles comprises an additional single dose of mosunetuzumab. In some embodiments, the additional single dose of mosunetuzumab is administered on Day 1 of the respective additional dosing cycle. In some embodiments, the additional single dose of mosunetuzumab is about 45 mg (e.g., 45 mg±10%; 45 mg±4.5 mg).

In one aspect, the invention features a method of treating an individual having SLE, comprising subcutaneously administering to the individual mosunetuzumab according to a dosing regimen comprising nine 21-day dosing cycles, wherein: (a) the first dosing cycle comprises a first dose (C1D1), a second dose (C1D2), and a third dose (C1D3), wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg), and wherein the C1D1, the C1D2, and the C1D3 are administered on Days 1, 8, and 15, respectively, of the first dosing cycle; and (b) the second to ninth dosing cycles each comprises a single dose (C2D1-C9D1) of mosunetuzumab administered on Day 1 of each respective dosing cycle, wherein each of the C2D1-C9D1 is about 45 mg (e.g., 45 mg±10%; 45 mg±4.5 mg).

In some embodiments, mosunetuzumab is administered as a monotherapy.

In some embodiments, prior to administration of any dose of mosunetuzumab, the individual has been previously administered an oral corticosteroid, an antimalarial agent, or an immunosuppressant. In some embodiments, the individual has been administered an antimalarial agent at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the antimalarial agent is hydroxychloroquine, chloroquine, or quinacrine. In some embodiments, the individual has been administered an immunosuppressant at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the immunosuppressant is azathioprine, mycophenolate mofetil, mycophenolic acid, or methotrexate.

In some embodiments, the individual does not have a lupus-associated neuropsychiatric disease. In some embodiments, the lupus-associated neuropsychiatric disease is meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, or mononeuritis multiplex.

In some embodiments, the individual does not have active overlap syndrome with mixed connective tissue disease or systemic sclerosis within a year prior to being administered mosunetuzumab. In some embodiments, the individual does not have catastrophic or severe antiphospholipid syndrome within a year prior to being administered mosunetuzumab, unless the severe antiphospholipid syndrome has been adequately controlled by administering anticoagulant therapy to the individual at least 2 months (e.g., 2, 3, 4, 5, 6, 7, 8, 9, 10, 11, 12, 18, 24, 30, 36 months, or more) prior to being administered mosunetuzumab.

In some embodiments, the individual has not been administered: (a) at least 12 months (e.g., 12, 18, 24, 30, 36, 42, 48 months, or more) prior to being administered mosunetuzumab: (i) an anti-CD19 antibody therapy, or (ii) an anti-CD20 monoclonal antibody therapy; (b) at least 30 days (e.g., 30, 60, 90, 120, 150, 180 days or more) prior to being administered mosunetuzumab: (i) kinase inhibitors of Janus Kinase (JAK) kinase, Bruton tyrosine kinase, or tyrosine kinase 2, or (ii) a calcineurin inhibitor; or (c) at least 2 months (e.g., 2, 3, 4, 5, 6, 7, 8, 9, 10, 11, 12, 18, 24, 30, 36 months, or more) prior to being administered mosunetuzumab: (i) cyclophosphamide, or (ii) a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitors are baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, the individual does not have significant lupus-associated renal disease or significant renal impairment.

In some embodiments, the individual does not have any of the following laboratory parameters: (a) total bilirubin >1.5×upper limit of normal (ULN); (b) absolute neutrophil count (ANC)<1.5×10⁹/L (<1500/μL); (c) platelet count <75×10⁹/L (100,000/mm³); (d) hemoglobin <90 g/L; (e) estimated glomerular filtration rate (eGFR)<30 ml/min/1.73 m² calculated according to the Chronic Kidney Disease Epidemiology Collaboration (CKD-EPI) equation; or (f) aspartate transferase (AST) or alanine transaminase (ALT) >2.5×ULN.

In some embodiments, the individual achieves an evaluable response after completion of the dosing regimen. In some embodiments, the evaluable response is remission, a clinical response, a complete renal response (CRR), or a partial renal response. In some embodiments, the evaluable response is remission, and wherein the individual: (a) maintains a clinical SLE Disease Activity Index=0 for 6 months after administration of the final dose of mosunetuzumab; and (b) is not administered any additional therapeutic agents for treating SLE, except for an antimalarial agent, for 6 months after administration of the final dose of mosunetuzumab. In some embodiments, the antimalarial agent is hydroxychloroquine, chloroquine, or quinacrine. In some embodiments, the individual is not administered any additional therapeutic agents for treating SLE, for 6 months after administration of the final dose of mosunetuzumab. In some embodiments, the evaluable response is a clinical response, and wherein the individual: (a) maintains a clinical SLE Disease Activity Index (cSLEDAI)=0 for 6 months after administration of the final dose of mosunetuzumab; and (b) is administered a stable dosing of SLE therapy for 6 months after administration of the final dose of mosunetuzumab; and (c) is administered ≤5 mg/day prednisone or equivalent for 6 months after administration of the final dose of mosunetuzumab. In some embodiments, the evaluable response is a complete renal response, wherein the individual has: (a) urine protein creatinine ratio (UPCR)<0.5 g/g; and (b) eGFR >85% of baseline or >60 ml/min/1.73 m² calculated according to the CKD-EPI equation. In some embodiments, the evaluable response is a partial renal response, wherein the individual has: (a) >50% reduction in UPCR from baseline; (b) UPCR <1 g/g, or UPCR <3 g/g if baseline UPCR was >3 g/g; and (c) eGFR >85% of baseline calculated according to the CKD-EPI equation.

In some embodiments, the individual exhibits at least one reduced symptom of SLE as measured using a Patient Global Impression of Severity (PGI-S), a Physician Global Assessment (PGA), a decrease in titer of antinuclear antibody (ANA), a decrease in titer of anti-double stranded DNA (dsDNA) antibody (IgG) titer, an increase in complement C3 levels, or an increase in complement C4 levels after completion of the dosing regimen. In some embodiments, the at least one reduced symptom is a change in response of at least one step on the PGI-S from a previous response, where the change is one change of from “very severe” to “severe,” “severe” to “moderate,” “moderate” to “mild,” or “mild” to “none.” In some embodiments, the at least one reduced symptom is a change in rating by a healthcare provider or other appropriately trained professional using the PGA, where the change is a decrease from a previous rating using the PGA. In some embodiments, the individual exhibits a decrease in titer of anti-nuclear antibody (ANA). In some embodiments, the individual exhibits a decrease in titer of anti-dsDNA antibody (IgG) titer. In some embodiments, the individual exhibits an increase in complement C3 levels. In some embodiments, the individual exhibits an increase in complement C4 levels.

In some embodiments, the individual is further administered tocilizumab if the individual experiences cytokine release syndrome (CRS).

In some embodiments, any of the methods herein above further comprises administering to the individual a corticosteroid or a calcineurin inhibitor. In some embodiments, (a) the corticosteroid comprises hydrocortisone, cortisone acetate, prednisolone, dexamethasone, betamethasone, triamcinolone, beclomethasone, methylprednisolone or prednisone; or (b) the calcineurin inhibitor comprises ciclosporin, tacrolimus, or vocolosporin.

In some embodiments, the individual has active Class III or IV lupus nephritis (LN), as defined by the 2018 International Society of Nephrology/Renal Pathology Society (ISN/RPS) classification guidelines (Bajema et al., Kidney International. 93(4):389-796, 2018). In some embodiments, the Class III or IV LN was determined using a renal biopsy sample. In some embodiments, the renal biopsy sample was determined to have ≥3 points on the NIH activity index, excluding any contribution from interstitial activity; and <50% glomerulosclerosis and <50% tubulointerstitial fibrosis.

In some embodiments, the individual has: (a) UPCR ≥1.15 g/g; (b) Complement C3 below the lower limit of normal (e.g., below 90 mg/dL, e.g., in a patient over 17 years old); and/or (c) active LN that has been treated for at least 6 months with standard nonsteroidal LN therapy prior to administration of mosunetuzumab. In some embodiments, the individual has Complement C4 below the lower limit of normal (e.g., below 12 mg/dL, e.g., in a patient over 17 years old). In some embodiments, the individual is positive (e.g., >75 IU/mL), negative (e.g., <30 IU/mL) or borderline (e.g., 30-75 IU/mL) for anti-double-stranded DNA antibody. In some embodiments, the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitor is baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, the individual does not have LN.

In some embodiments, the individual has autoantibody positivity, hypocomplementemia, and high disease activity despite treatment with advanced therapy. In some embodiments, has an anti-double-stranded DNA antibody or anti-Smith antibody above ULN; or a Complement C3 level below the lower limit of normal. In some embodiments, the individual has an Extrarenal SLE Disease Activity Index 2000 (SLEDAI-2K) score >8, excluding points for: (a) proteinuria, pyuria, hematuria, casts, alopecia, fever, and headache; (b) rash if Cutaneous LE Disease Area and Severity Index (CLASI; Albrecht et al., J Invest Dermatol. 125(5):889-894, 2005) activity score is <6; (c) mucosal ulcers that do not significantly interfere with oral intake; and (d) arthritis with <4 joints that are both swollen and tender or with <2 located in the wrist or hand. In some embodiments, the advanced therapy comprises treatment for at least 6 months with cyclophosphamide, belimumab, ustekinumab, anifrolumab, secukinumab, atacicept, obinutuzumab, rituximab, ocrelizumab, or ofatumumab.

In some embodiments, the individual is administered dexamethasone, diphenhydramine, and/or acetaminophen/paracetamol prior to administration of any dose of mosunetuzumab. In some embodiments, the individual is administered dexamethasone, diphenhydramine, and acetaminophen/paracetamol prior to administration of the C1D1, C1D2, C1D3, and C2D1 of mosunetuzumab. In some embodiments, dexamethasone is administered orally at a dose of about 20 mg (e.g., 20 mg±10%; 20 mg±2 mg); diphenhydramine is administered orally at a dose of about 25 mg to about 50 mg (e.g., 25 mg to 45 mg, 25 mg to 40 mg, 25 mg to 35 mg, 25 mg to 30 mg, 30 mg to 50 mg, 40 mg to 50 mg, 45 mg to 50 mg, 30 mg to 35 mg, 30 mg to 45 mg, 35 mg to 45 mg, 40 mg to 45 mg, or 30 mg to 40 mg; e.g., about 25 mg, about 30 mg, about 35 mg, about 40 mg, about 45 mg, or about 50 mg); and acetaminophen/paracetamol is administered orally at a dose of about 650 mg to about 1000 mg (e.g., 650 mg to 900 mg, 650 mg to 80 mg, 650 mg to 700 mg, 700 mg to 1000 mg, 800 mg to 1000 mg, 900 mg to 1000 mg, 700 mg to 800 mg, 800 mg to 900 mg, 700 mg to 900 mg; e.g., about 650 mg, about 700 mg, about 750 mg, about 800 mg, about 850 mg, about 900 mg, about 950 mg, or about 1000 mg).

In one aspect, the invention features a method of treating a population of individuals having SLE, comprising subcutaneously administering to each individual of the population mosunetuzumab according to a dosing regimen comprising at least a first dosing cycle and a second dosing cycle, wherein: (a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg± 4.5 mg); and (b) the second dosing cycle comprises a single dose (C2D1) of mosunetuzumab, wherein the C2D1 of mosunetuzumab is about 45 mg (e.g., 45 mg±10%; 45 mg±4.5 mg).

In some embodiments, the first dosing cycle is a 21-day dosing cycle. In some embodiments, the C1D1, C1D2, and C1D3 are administered on or about Days 1, 8, and 15, respectively, of the first dosing cycle. In some embodiments, the second dosing cycle is a 21-day dosing cycle. In some embodiments, the C2D1 is administered on or about Day 1 of the second dosing cycle.

In some embodiments, the method further comprises one or more additional dosing cycles. In some embodiments, the method comprises one to seven (e.g., one, two, three, four, five, six, or seven) additional dosing cycles. In some embodiments, the method comprises seven additional dosing cycles. In some embodiments, each additional dosing cycle is a 21-day dosing cycle. In some embodiments, one or more of the additional dosing cycles comprises an additional single dose of mosunetuzumab. In some embodiments, the additional single dose of mosunetuzumab is administered on Day 1 of the respective additional dosing cycle. In some embodiments, the additional single dose of mosunetuzumab is about 45 mg (e.g., 45 mg±10%; 45 mg±4.5 mg).

In one aspect, the invention features a method of treating a population of individuals having SLE, comprising subcutaneously administering to each individual of the population mosunetuzumab according to a dosing regimen comprising nine 21-day dosing cycles, wherein: (a) the first dosing cycle comprises a first dose (C1D1), a second dose (C1D2), and a third dose (C1D3), wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg), and wherein the C1D1, the C1D2, and the C1D3 are administered on Days 1, 8, and 15, respectively, of the first dosing cycle; and (b) the second to ninth dosing cycles each comprises a single dose (C2D1-C9D1) of mosunetuzumab administered on Day 1 of each respective dosing cycle, wherein each of the C2D1-C9D1 is about 45 mg (e.g., 45 mg±10%; 45 mg±4.5 mg).

In some embodiments, mosunetuzumab is administered as a monotherapy.

In some embodiments, prior to administration of any dose of mosunetuzumab, an individual of the population has been previously administered an oral corticosteroid, an antimalarial agent, or an immunosuppressant. In some embodiments, an individual of the population has been administered an antimalarial agent at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the antimalarial agent is hydroxychloroquine, chloroquine, or quinacrine. In some embodiments, an individual of the population has been administered an immunosuppressant at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the immunosuppressant is azathioprine, mycophenolate mofetil, mycophenolic acid, or methotrexate.

In some embodiments, an individual of the population does not have a lupus-associated neuropsychiatric disease. In some embodiments, the lupus-associated neuropsychiatric disease is meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, or mononeuritis multiplex.

In some embodiments, an individual of the population does not have active overlap syndrome with mixed connective tissue disease or systemic sclerosis within a year prior to being administered mosunetuzumab. In some embodiments, an individual of the population does not have catastrophic or severe antiphospholipid syndrome within a year prior to being administered mosunetuzumab, unless the severe antiphospholipid syndrome has been adequately controlled by administering anticoagulant therapy to the individual at least 2 months (e.g., 2, 3, 4, 5, 6, 7, 8, 9, 10, 11, 12, 18, 24, 30, 36 months, or more) prior to being administered mosunetuzumab.

In some embodiments, an individual of the population has not been administered: (a) at least 12 months (e.g., 12, 18, 24, 30, 36, 42, 48 months, or more) prior to being administered mosunetuzumab: (i) an anti-CD19 antibody therapy, or (ii) an anti-CD20 monoclonal antibody therapy; (b) at least 30 days (e.g., 30, 60, 90, 120, 150, 180 days or more) prior to being administered mosunetuzumab: (i) kinase inhibitors of Janus Kinase (JAK) kinase, Bruton tyrosine kinase, or tyrosine kinase 2, or (ii) a calcineurin inhibitor; or (c) at least 2 months (e.g., 2, 3, 4, 5, 6, 7, 8, 9, 10, 11, 12, 18, 24, 30, 36 months, or more) prior to being administered mosunetuzumab: (i) cyclophosphamide, or (ii) a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitors are baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, an individual of the population does not have significant lupus-associated renal disease or significant renal impairment.

In some embodiments, an individual of the population does not have any of the following laboratory parameters: (a) total bilirubin >1.5×upper limit of normal (ULN); (b) absolute neutrophil count (ANC)<1.5×10⁹/L (<1500/μL); (c) platelet count <75×10⁹/L (100,000/mm³); (d) hemoglobin <90 g/L; (e) estimated glomerular filtration rate (eGFR)<30 ml/min/1.73 m² calculated according to the Chronic Kidney Disease Epidemiology Collaboration (CKD-EPI) equation; or (f) aspartate transferase (AST) or alanine transaminase (ALT) >2.5×ULN.

In some embodiments, an individual of the population achieves an evaluable response after completion of the dosing regimen. In some embodiments, the evaluable response is remission, a clinical response, a complete renal response (CRR), or a partial renal response. In some embodiments, the evaluable response is remission, and wherein an individual of the population: (a) maintains a clinical SLE Disease Activity Index=0 for 6 months after administration of the final dose of mosunetuzumab; and (b) is not administered any additional therapeutic agents for treating SLE, except for an antimalarial agent, for 6 months after administration of the final dose of mosunetuzumab. In some embodiments, the antimalarial agent is hydroxychloroquine, chloroquine, or quinacrine. In some embodiments, an individual of the population is not administered any additional therapeutic agents for treating SLE, for 6 months after administration of the final dose of mosunetuzumab. In some embodiments, the evaluable response is a clinical response, and wherein an individual of the population: (a) maintains a clinical SLE Disease Activity Index (cSLEDAI)=0 for 6 months after administration of the final dose of mosunetuzumab; and (b) is administered a stable dosing of SLE therapy for 6 months after administration of the final dose of mosunetuzumab; and (c) is administered ≤5 mg/day prednisone or equivalent for 6 months after administration of the final dose of mosunetuzumab. In some embodiments, the evaluable response is a complete renal response, wherein an individual of the population has: (a) urine protein creatinine ratio (UPCR)<0.5 g/g; and (b) eGFR ≥85% of baseline or ≥60 ml/min/1.73 m² calculated according to the CKD-EPI equation. In some embodiments, the evaluable response is a partial renal response, wherein an individual of the population has: (a) ≥50% reduction in UPCR from baseline; (b) UPCR <1 g/g, or UPCR <3 g/g if baseline UPCR was ≥3 g/g; and (c) eGFR ≥85% of baseline calculated according to the CKD-EPI equation.

In some embodiments, an individual of the population exhibits at least one reduced symptom of SLE as measured using a Patient Global Impression of Severity (PGI-S), a Physician Global Assessment (PGA), a decrease in titer of antinuclear antibody (ANA), a decrease in titer of anti-double stranded DNA (dsDNA) antibody (IgG) titer, an increase in complement C3 levels, or an increase in complement C4 levels after completion of the dosing regimen. In some embodiments, the at least one reduced symptom is a change in response of at least one step on the PGI-S from a previous response, where the change is one change of from “very severe” to “severe,” “severe” to “moderate,” “moderate” to “mild,” or “mild” to “none.” In some embodiments, the at least one reduced symptom is a change in rating by a healthcare provider or other appropriately trained professional using the PGA, where the change is a decrease from a previous rating using the PGA. In some embodiments, an individual of the population exhibits a decrease in titer of anti-nuclear antibody (ANA). In some embodiments, an individual of the population exhibits a decrease in titer of anti-dsDNA antibody (IgG) titer. In some embodiments, an individual of the population exhibits an increase in complement C3 levels. In some embodiments, an individual of the population exhibits an increase in complement C4 levels.

In some embodiments, an individual of the population is further administered tocilizumab if the individual experiences cytokine release syndrome (CRS).

In some embodiments, any of the methods herein above further comprises administering to an individual of the population a corticosteroid or a calcineurin inhibitor. In some embodiments, (a) the corticosteroid comprises hydrocortisone, cortisone acetate, prednisolone, dexamethasone, betamethasone, triamcinolone, beclomethasone, methylprednisolone or prednisone; or (b) the calcineurin inhibitor comprises ciclosporin, tacrolimus, or vocolosporin.

In some embodiments, an individual of the population has active Class III or IV lupus nephritis (LN), as defined by the 2018 International Society of Nephrology/Renal Pathology Society (ISN/RPS) classification guidelines (Bajema et al., Kidney International. 93(4):389-796, 2018). In some embodiments, the Class III or IV LN was determined using a renal biopsy sample. In some embodiments, the renal biopsy sample was determined to have ≥3 points on the NIH activity index, excluding any contribution from interstitial activity; and <50% glomerulosclerosis and <50% tubulointerstitial fibrosis.

In some embodiments, an individual of the population has: (a) UPCR ≥1.15 g/g; (b) Complement C3 below the lower limit of normal (e.g., below 90 mg/dL, e.g., in a patient over 17 years old); and/or (c) active LN that has been treated for at least 6 months with standard nonsteroidal LN therapy prior to administration of mosunetuzumab. In some embodiments, the individual has Complement C4 below the lower limit of normal (e.g., below 12 mg/dL, e.g., in a patient over 17 years old). In some embodiments, the individual is positive (e.g., >75 IU/mL), negative (e.g., <30 IU/mL) or borderline (e.g., 30-75 IU/mL) for anti-double-stranded DNA antibody. In some embodiments, the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitor is baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, an individual of the population does not have LN.

In some embodiments, an individual of the population has autoantibody positivity, hypocomplementemia, and high disease activity despite treatment with advanced therapy. In some embodiments, has an anti-double-stranded DNA antibody or anti-Smith antibody above ULN; or a Complement C3 level below the lower limit of normal. In some embodiments, an individual of the population has an Extrarenal SLE Disease Activity Index 2000 (SLEDAI-2K) score ≥8, excluding points for: (a) proteinuria, pyuria, hematuria, casts, alopecia, fever, and headache; (b) rash if Cutaneous LE Disease Area and Severity Index (CLASI; Albrecht et al., J Invest Dermatol. 125(5):889-894, 2005) activity score is <6; (c) mucosal ulcers that do not significantly interfere with oral intake; and (d) arthritis with <4 joints that are both swollen and tender or with <2 located in the wrist or hand. In some embodiments, the advanced therapy comprises treatment for at least 6 months with cyclophosphamide, belimumab, ustekinumab, anifrolumab, secukinumab, atacicept, obinutuzumab, rituximab, ocrelizumab, or ofatumumab.

In some embodiments, an individual of the population is administered dexamethasone, diphenhydramine, and/or acetaminophen/paracetamol prior to administration of any dose of mosunetuzumab. In some embodiments, an individual of the population is administered dexamethasone, diphenhydramine, and acetaminophen/paracetamol prior to administration of the C1D1, C1D2, C1D3, and C2D1 of mosunetuzumab. In some embodiments, dexamethasone is administered orally at a dose of about 20 mg (e.g., 20 mg±10%; 20 mg±2 mg); diphenhydramine is administered orally at a dose of about 25 mg to about 50 mg (e.g., 25 mg to 45 mg, 25 mg to 40 mg, 25 mg to 35 mg, 25 mg to 30 mg, 30 mg to 50 mg, 40 mg to 50 mg, 45 mg to 50 mg, 30 mg to 35 mg, 30 mg to 45 mg, 35 mg to 45 mg, 40 mg to 45 mg, or 30 mg to 40 mg; e.g., about 25 mg, about 30 mg, about 35 mg, about 40 mg, about 45 mg, or about 50 mg); and acetaminophen/paracetamol is administered orally at a dose of about 650 mg to about 1000 mg (e.g., 650 mg to 900 mg, 650 mg to 80 mg, 650 mg to 700 mg, 700 mg to 1000 mg, 800 mg to 1000 mg, 900 mg to 1000 mg, 700 mg to 800 mg, 800 mg to 900 mg, 700 mg to 900 mg; e.g., about 650 mg, about 700 mg, about 750 mg, about 800 mg, about 850 mg, about 900 mg, about 950 mg, or about 1000 mg).

In some embodiments, mosunetuzumab is administered to the individual or the individuals of the population as a pharmaceutical composition comprising about 1 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some embodiments, mosunetuzumab is administered to the individual or the individuals of the population as a pharmaceutical composition comprising about 10 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some embodiments, mosunetuzumab is administered to the individual or the individuals of the population as a pharmaceutical composition comprising about 45 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some embodiments, the pharmaceutical composition further comprises histidine acetate.

In some embodiments, administering one or more doses of mosunetuzumab to the individual or individuals induces a B cell depletion in the individual or individuals. In some embodiments, the B cell depletion is a deep-tissue B cell depletion and/or a peripheral B cell depletion. In some embodiments, the deep-tissue B cell depletion is determined by tissue biopsy. In some embodiments, the deep-tissue B cell depletion is determined by kidney biopsy. In some embodiments, the peripheral B cell depletion is determined by blood flow cytometry. In some embodiments, the amount of B cell depletion is determined relative to a reference level of B cells. In some embodiments, the reference level of B cells is determined at a time prior to administration of any dose of mosunetuzumab. In some embodiments, the method further comprises discontinuing administering a background immunotherapy to the individual. In some embodiments, the background immunotherapy comprises at least one selected from the group consisting of mycophenolate mofetil, azathioprine, and methotrexate.

In one aspect, the invention features a method of inducing B cell depletion in an individual, comprising subcutaneously administering to the individual mosunetuzumab according to a dosing regimen comprising nine 21-day dosing cycles, wherein: (a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are administered on Days 1, 8, and 15, respectively, of the first dosing cycle; and (b) the second to ninth dosing cycles each comprises a single dose (C2D1-C9D1) of mosunetuzumab administered on Day 1 of each respective dosing cycle, wherein each of the C2D1-C9D1 is about 45 mg.

In some embodiments, the B cell depletion is a deep-tissue B cell depletion and/or a peripheral B cell depletion. In some embodiments, the deep-tissue B cell depletion is determined by tissue biopsy. In some embodiments, the deep-tissue B cell depletion is determined by kidney biopsy. In some embodiments, the peripheral B cell depletion is determined by blood flow cytometry. In some embodiments, the amount of B cell depletion is determined relative to a reference level of B cells. In some embodiments, the reference level of B cells is determined at a time prior to administration of any dose of mosunetuzumab. In some embodiments, the individual has SLE. In some embodiments, the method further comprises discontinuing administering a background immunotherapy to the individual. In some embodiments, the background immunotherapy comprises at least one selected from the group consisting of mycophenolate mofetil, azathioprine, and methotrexate.

In one aspect, the invention features a method of treating an individual having systemic lupus erythematosus (SLE), comprising subcutaneously administering to the individual mosunetuzumab according to a dosing regimen comprising at least a first dosing cycle and a second dosing cycle, wherein: (a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and (b) the second dosing cycle comprises a single dose (C2D1) of mosunetuzumab, wherein the C2D1 of mosunetuzumab is about 45 mg, and wherein the method further comprises discontinuing administering a background immunotherapy to the individual.

In some embodiments, the individual has B cell depletion after dosing the individual with mosunetuzumab before reducing or removing background immunotherapy to the individual. In some embodiments, the B cell depletion is a deep-tissue B cell depletion and/or a peripheral B cell depletion. In some embodiments, the deep-tissue B cell depletion is determined by tissue biopsy. In some embodiments, the deep-tissue B cell depletion is determined by kidney biopsy. In some embodiments, the peripheral B cell depletion is determined by blood flow cytometry. In some embodiments, the amount of B cell depletion is determined relative to a reference level of B cells. In some embodiments, the reference level of B cells is determined at a time prior to administration of any dose of mosunetuzumab. In some embodiments, the background immunotherapy comprises at least one selected from the group consisting of mycophenolate mofetil, azathioprine, and methotrexate.

### BRIEF DESCRIPTION OF THE DRAWINGS

FIG. 1 is a schematic showing the scheme of the Phase II study. Dose 1=1.6 mg (Day 1); Dose 2=5 mg (Day 8); Dose 3=45 mg (Day 15); Doses 4-11=45 mg (Every 3 weeks starting on Day 22). cSLEDAI=Clinical Systemic Lupus Erythematosus Disease Activity Index; SFU=safety follow-up (required until peripheral B cells return); SLE=systemic lupus erythematosus. Disease-modifying antirheumatic drugs (DMARDs) and other immunosuppressive therapies, including steroids, must be discontinued to meet primary endpoint (antimalarials remain permitted).

FIG. 2 is a schematic showing the scheme of the Phase Ib study. n=number of patients; Gr3 CRS=Grade 3 cytokine release syndrome; SC=subcutaneous.

FIG. 3 is a set of graphs showing Systemic Lupus Erythematosus Disease Activity Index 2000 (SLEDAI-2K) scores of patients prior to and after administration of mosunetuzumab. SLEDAI-2K=Systemic Lupus Erythematosus Disease Activity Index 2000; SC=subcutaneous; MMF=mycophenolate mofetil; 1.6 mg=1.6 mg of mosunetuzumab administered on Day 1; 5 mg=5 mg of mosunetuzumab administered on Day 1; 5+15 mg=5 mg of mosunetuzumab administered on Day 1 and 15 mg of mosunetuzumab administered on Day 8; 5+45 mg=5 mg of mosunetuzumab administered on Day 1 and 45 mg of mosunetuzumab administered on Day 8; and 5+60 mg=5 mg of mosunetuzumab administered on Day 1 and 60 mg of mosunetuzumab administered on Day 8. No clear evidence of dose response was observed.

FIG. 4 is a set of graphs showing the concentration of cluster of differentiation-positive (CD19+) cells in blood following treatment with mosunetuzumab. Arrows denote administration of mosunetuzumab. The dashed line indicates the lower limit of quantification of 0.4 cells/μL. SC=subcutaneous; MMF=mycophenolate mofetil; LLOQ=lower limit of quantification; BLOQ=below limit of quantification; 1.6 mg=1.6 mg of mosunetuzumab administered on Day 1; 5 mg=5 mg of mosunetuzumab administered on Day 1; 5+15 mg=5 mg of mosunetuzumab administered on Day 1 and 15 mg of mosunetuzumab administered on Day 8; 5+45 mg=5 mg of mosunetuzumab administered on Day 1 and 45 mg of mosunetuzumab administered on Day 8; and 5+60 mg=5 mg of mosunetuzumab administered on Day 1 and 60 mg of mosunetuzumab administered on Day 8. Results below LLOQ were imputed as ½ LLOQ, or 0.2205 cells/μL. No clear evidence of dose response was observed for patients with baseline score ≥8.

FIG. 5 is a graph showing anti-dsDNA antibody levels in ten patients administered mosunetuzumab. Patients are grouped as per actual dose received, regardless of their original cohort assignment. One patient assigned to the 5+15 mg cohort and one patient assigned to the 5+45 mg cohort received only the Day 1 injection (5 mg) and did not receive their respective Day 8 injections. Results below limit of quantification (BLOQ) were imputed to ½ BLOQ. dsDNA: double-stranded DNA; IU: international units. Five lines marked with doses represent anti-dsDNA levels in the five patients with positive anti-dsDNA levels at baseline. All five patients had a diminution in positivity of their anti-dsDNA levels. The line marked “All pts” shows anti-dsDNA levels for the other ten patients who had a negative anti-dsDNA antibody level throughout the study, i.e., shows the results for 1 patient from 1.6 mg cohort, 4 patients from 5 mg cohort, 2 patients from 5+15 mg cohort, 2 patients from 5+45 mg cohort, and 1 patient from 5+60 mg cohort.

FIG. 6 is a set of graphs showing the percent of cluster of differentiation 4-positive (CD4+; T-helper) cells that are cluster of differentiation 69-positive (CD69+; top graphs) and the percent of cluster of differentiation 8-positive (CD8+; T-effector) cells that are cluster of differentiation 69-positive (CD69+; bottom graphs) after administration of mosunetuzumab. Arrows denote administration of mosunetuzumab. SC=subcutaneous; 1.6 mg=1.6 mg of mosunetuzumab administered on Day 1; 5 mg=5 mg of mosunetuzumab administered on Day 1; 5+15 mg=5 mg of mosunetuzumab administered on Day 1 and 15 mg of mosunetuzumab administered on Day 8; 5+45 mg=5 mg of mosunetuzumab administered on Day 1 and 45 mg of mosunetuzumab administered on Day 8; and 5+60 mg=5 mg of mosunetuzumab administered on Day 1 and 60 mg of mosunetuzumab administered on Day 8.

### DETAILED DESCRIPTION

The present invention relates to methods of treating an individual having systemic lupus erythematosus (SLE) by subcutaneous administration of mosunetuzumab using a specific dosing regimen that increases patient safety and increases the probability that the individual having SLE will respond to the greater reduction/elimination of B-cells as compared to other antibody-oriented treatments (excluding CAR-T cell therapies). In some embodiments, the treatment comprises subcutaneous administration of mosunetuzumab as a monotherapy, e.g., for the treatment of SLE.

Standard-of-care therapies for SLE include oral corticosteroids (OCS), antimalarial therapies (such as hydroxychloroquine, chloroquine, and quinacrine), conventional immunosuppressants (such as mycophenolate mofetil [MMF], azathioprine, and methotrexate), and monoclonal antibodies (such as belimumab and anifrolumab). However, many patients adherent to this multiple-therapy regimen still often experience flares of active disease and thus remain vulnerable to untreated inflammation. Additionally, prolonged treatment with standard therapies carries a significant risk of medication toxicity, a variety of steroid side effects, and chronic immunosuppression that can lead to an increased susceptibility to serious infection. The methods disclosed herein aim to avoid the risks associated with prolonged treatment with standard therapies.

Methods described herein comprise subcutaneously administering mosunetuzumab to an individual, e.g., of a population, according to a dosing regimen comprising at least a first dosing cycle and a second dosing cycle, wherein: (a) the first dosing cycle comprises a first dose (C1D1), a second dose (C1D2), and a third dose (C1D3) of mosunetuzumab, (b) the second dosing cycle comprises a first dose (C2D1) of mosunetuzumab. In particular, the first dosing cycle is a 21-day (±1 day) dosing cycle and the C1D1, C1D2, and C1D3 doses are administered on or about Days 1, 8 (±1 day), and 15 (±1 day), respectively, and the second dosing cycle is a 21-day (±1 day) dosing cycle and the C2D1 is administered on Day 1. Methods of the invention may additionally include administration of additional therapeutic agents, such as premedication (e.g., with a corticosteroid, an antihistamine, and/or an anti-pyretic).

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

It is to be understood that aspects and embodiments of the invention described herein include “comprising,” “consisting,” and “consisting essentially of” aspects and embodiments.

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” a value or parameter refers to that value or parameter ±10%.

A “disorder” is any condition that would benefit from treatment including, but not limited to, chronic and acute disorders or diseases including those pathological conditions which predispose the mammal to the disorder in question.

“Systemic lupus erythematosus” or “SLE” refers to an autoimmune rheumatic disease that occurs primarily in women of childbearing age. It is characterized by multisystem involvement and immunological abnormalities, including dysfunction of B cells and T cells. Much of the tissue damage is thought to occur through autoantibody formation and immune complex deposition, but the underlying cause remains unknown. Systemic lupus erythematosus is an unpredictable disease that is characterized by alternating periods of remission and a wide variety of clinical manifestations that may affect any organ (Fortuna G, Brennan M T. Systemic lupus erythematosus: epidemiology, pathophysiology, manifestations, and management. Dent Clin North Am. 2013; 57(4):631-655). Accordingly, there are two major categories: (1) constitutional and (2) organ sign/symptoms. The constitutional category includes fatigue (80-100% of all patients), which appears correlated to depression, stress, anemia, smoking habits, sedentary lifestyle, sleep, and coexistent fibromyalgia. In addition, fever is observed in about 50% of systemic lupus erythematosus patients, which may be the result of the disease, a drug reaction, or a systemic lupus erythematosus-induced infection. Finally weight loss is often observed before diagnosis of systemic lupus erythematosus; observed weight gain after diagnosis seems to relate to the side effects of corticosteroid treatments.

Organ signs/symptoms are more numerous. They include:
Musculoskeletal: arthralgia (often migratory), arthritis (often migratory), osteonecrosis avascular necrosis of bone and myopathy.
Renal: due to direct damage from systemic lupus erythematosus to the kidney. These include lupus nephritis, which is responsible for a significant morbidity and mortality. Forms of lupus nephritis include glomerulonephritis; other lesions can include renal amyloidosis, focal segmental glomerulosclerosis, IgA and IgM nephropathy, and necrotizing glomerulitis.
Gastrointestinal: mesenteric vasculitis and thrombosis; protein-losing enteropathy, intestinal pseudo-obstruction, and acute pancreatitis; and rarely, celiac disease, inflammatory bowel diseases, eosinophilic enteritis, and pneumatosis cystoides intestinalis.
Pulmonary: pleuritis is most common; sometimes observed are pneumonitis, acute respiratory distress, diffuse alveolar hemorrhage, chronic interstitial pneumonitis, and shrinking (vanishing) lung syndrome. More rare is acute reversible hypoxemia, pulmonary embolisms, pulmonary arterial hypertension, and airway disease obstructive lung disease and upper airways disease.
Cardiovascular: valvular heart diseases associated with Libman-Sacks disease lesions, sterile vegetations, serositis associated with pericardial disease, and venous and arterial thrombosis associated with antiphospholipid antibodies are sometimes observed.
Neuropsychiatric: seizures and cerebrovascular disease, such as stroke, transient ischemic attack, and venous sinus thrombosis; psychiatric manifestations include depression and cognitive dysfunction. Additional such symptoms can present, including headaches, mood disorders, acute confusional states, and anxiety.
Hematological: cytopenia and thrombophilia. Systemic lupus erythematosus patients may also present with anemia, thrombocytopenia, neutropenia, and leukopenia.
Ocular: About a third of systemic lupus erythematosus patients will have ocular involvement, including that of the optic nerve, periorbita, and ocular adnex, the most common manifestation is keratoconjunctivitis sicca. Ocular damage can harm visual acuity.
Cutaneous: 85% of systemic lupus erythematosus patients present with cutaneous symptoms and may be the only involve organ. Three groups of mucocutaneous manifestations can present: (1) chronic cutaneous lupus erythematosus (CCLE), (2) subacute cutaneous lupus erythematosus (SCLE), and (3) acute cutaneous lupus erythematosus (ACLE).
Cutaneous manifestations include malar rash, discoid lesions (localized or generalized), photosensitivity, papular/plaque lesions with slight scaling, alopecia, deep brawny indurations or subcutaneous nodule, atrophie blanche, and livedo reticularis.
Oropharyngeal: in addition to lesions, oral candidiasis, dysphagia, and (perhaps) xerostomia.
Immunologic: antinuclear antibody (ANA) titers that are abnormal, either above a laboratory reference range or as determined by immunofluorescence or equivalent assay. Antibodies recognizing native (ds) DNA assays show results above a laboratory reference range (if ELISA, usually twice the above laboratory reference range is used). Present of antibody to the SM nuclear antigen (anti-SM antibodies). Anti-phospholipid antibodies, which can be determined by a lupus anticoagulant test; false-positive test for syphilis; anticardiolipin antibody (at least twice normal or medium-high titer); anti-b2 glycoprotein 1. Low levels complement C3, C4, or CH50 (i.e., total complement level; Ayano M, Horiuchi T. Biomolecules. February 15; 13(2):367, 2023) can be present. Finally, a positive Direct Coombs test, in the absence of hemolytic anemia.

“Lupus nephritis” or “LN” is the most common target-organ manifestation of SLE. In some embodiments, LN can be classified into class I, II, III, IV, V, or VI according to the 2018 International Society of Nephrology/Renal Pathology Society (ISN/RPS) classification guidelines (Bajema et al., Kidney International. 93(4):389-796, 2018). In some embodiments, the classification is determined using a renal biopsy sample.

“Reducing at least one symptom of systemic lupus erythematosus” refers to in part reducing any of the above listed symptoms, whether they be constitutional or organ signs/symptoms.

“NIH activity index” refers to a semi-quantitative grading system of pathologic features on kidney biopsies, which allows for monitoring response to treatment and showing disease progression, as described in Bajema et al., Kidney Int. 93(4):789-796, 2018. Activity indices (scoring) include endocapillary hypercellularity (0-3); neutrophils/karyorrhexis (0-3); hyaline deposits/wire loops (0-3); fibrinoid necrosis (0-3×2); cellular or fibrocellular crescents (0-3×2); and interstitial inflammation (0-3), for a total score out of 24 possible.

“NIH chronicity index” refers to a semi-quantitative grading system of pathologic features on kidney biopsies, which allows for monitoring response to treatment and showing disease progression, as described in Bajema et al., Kidney Int. 93(4):789-796, 2018. Chronicity indices (scoring) include global glomerulosclerosis (0-3); fibrous crescents (0-3); tubular atrophy (0-3); and interstitial fibrosis (0-3), for a total score out of 12 possible.

“Lupus-associated neuropsychiatric diseases” include, but are not limited to meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, and mononeuritis multiplex.

“Overlap syndrome” refers to inflammatory rheumatic conditions. Most commonly involved in overlap syndromes are rheumatoid arthritis, scleroderma, lupus, and myositis. “Mixed connective tissue disease” refers to a manifestation of overlap syndrome that is defined by anti-U1 ribonucleoprotein autoimmunity with features of at least two of rheumatoid arthritis, lupus, scleroderma, and myositis.

“Antiphospholipid overlap syndrome” refers to an autoimmune disorder wherein anti-self phospholipid antibodies attack phospholipids. Symptoms vary and can include blood clots, miscarriage, rash, chronic headaches, dementia, and seizures. “Severe antiphospholipid overlap syndrome” is an antiphospholipid overlap syndrome that is difficult to treat with conventional therapies. “Catastrophic antiphospholipid overlap syndrome” refers to a rare form of antiphospholipid overlap syndrome where multiple blood clots form throughout the body over a brief time period, often resulting in organ failure if untreated. Antiphospholipid overlap syndrome is often treated with anticoagulant therapy.

“Anticoagulant therapies” can include, for example, (oral) vitamin K antagonists (e.g., warfarin) and direct oral anticoagulants (such as rivaroxaban, apixaban and dabigatran).

“CD19” refers to “cluster of differentiation 19” and is expressed in the whole B lineage from the pro B cell to the mature B cell, it is not shed, is uniformly expressed on all B lineage cells, and is absent from stem cells. “Anti-CD19 antibody therapy” indicates a therapy that administers an anti-CD19 antibody to a patient. An example of an anti-CD19 antibody is blinatumomab.

“Janus kinase inhibitor” refers to a compound that inhibits an activity of a Janus kinase (“JAK”), such as JAK1, JAK2, JAK3, and TYK2 (tyrosine kinase 2). Common Janus kinase inhibitors include baricitinib, tofacitinib, upadacitinib, filgotinib, ibrutinib, and fenebrutinib.

“Bruton tyrosine kinase inhibitor” refers to an inhibitor of Bruton tyrosine kinase activity. Examples include ibrutinib, acalabrutinib, and zanubrutinib.

As used herein, “treatment” (and grammatical variations thereof, such as “treat” or “treating”) refers to clinical intervention in an attempt to alter the natural course of the patient being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies are used to delay development of a disease or to slow the progression of a disease.

As used herein, administration or use of mosunetuzumab as a “monotherapy” refers to the use of mosunetuzumab alone to treat an individual having SLE, e.g., without administration or use of other therapeutic agents (e.g., standard-of-care therapies) for treating SLE. In some embodiments, the individual administered mosunetuzumab monotherapy does not receive treatment with an anti-CD20 antibody (e.g., obinutuzumab, rituximab, ocrelizumab, or ofatumumab), an anti-CD19 antibody (e.g., blinatumomab), CAR-T, a calcineurin inhibitor (e.g., tacrolimus, ciclosporin, or voclosporin), an oral corticosteroid (OCS), an antimalarial therapy (e.g., hydroxychloroquine, chloroquine, or quinacrine), a conventional immunosuppressants (e.g., mycophenolate mofetil (MMF), azathioprine, or methotrexate), cyclophosphamide, a kinase inhibitor (e.g., JAK kinase, Bruton tyrosine kinase, or tyrosine kinase 2 inhibitor; e.g., baricitinib, tofacitinib, upadacitinib, filgotinib, ibrutinib, or fenebrutinib); a calcineurin inhibitor; cyclophosphamide; or a biologic therapy (e.g., belimumab, ustekinumab, anifrolumab, secukinumab, or atacicept). Administration of mosunetuzumab as a monotherapy, e.g., for treating SLE, does not indicate that additional therapeutic agents for treating non-SLE indications and symptoms, may not be administered before, during, or after administration of mosunetuzumab. In particular, administration of mosunetuzumab as monotherapy permits the administration of additional therapeutic agents (e.g., tocilizumab, dexamethasone, diphenhydramine, and/or acetaminophen/paracetamol) for treatment of CRS events.

As used herein, “delaying progression” of a disorder or disease means to defer, hinder, slow, retard, stabilize, and/or postpone development of the disease or disorder (e.g., systemic lupus erythematosus). This delay can be of varying lengths of time, depending on the history of the disease and/or patient being treated. As is evident to one skilled in the art, a sufficient or significant delay can, in effect, encompass prevention, in that the patient does not develop the disease.

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, neutropenia, thrombocytopenia, elevated liver enzymes, and/or central nervous system (CNS) toxicities, following treatment with an anti-CD20/anti-CD3 bispecific antibody (e.g., mosunetuzumab). In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of systemic lupus erythematosus.

As used herein, “administering” is meant a method of giving a dosage of a compound (e.g., a bispecific antibody or an additional therapeutic agent described herein) or a composition (e.g., a pharmaceutical composition, e.g., a pharmaceutical composition including a bispecific antibody or an additional therapeutic agent described herein) to a patient. In the methods described herein, mosunetuzumab is administered subcutaneously (e.g., by subcutaneous injection). Additional therapeutic agents can be administered orally, intravenously (e.g., by intravenous infusion), or subcutaneously (e.g., by subcutaneous injection) according to the methods known in the art.

A “fixed dose” or “flat dose” refers to a dose of a therapeutic agent (e.g., mosunetuzumab) herein that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

“Stable dose” refers to a dose that is consistent for a period of time. The period of time can be as short as a few days and as long as many years. In opposition is an “adjustable dose.”

An “individual,” “participant,” or a “patient” is a mammal. Mammals include, but are not limited to, primates (e.g., humans and non-human primates such as monkeys), domesticated animals (e.g., cows, sheep, cats, dogs, and horses), rabbits, and rodents (e.g., mice and rats). In certain embodiments, the individual, participant, or patient is a human.

“Individual response,” “patient response,” or “response” refers to a response that can be assessed using any endpoint indicating a benefit to the individual or patient, including, without limitation, inhibition, to some extent, of disease progression. Responses to therapy or treatment of systemic lupus erythematosus can be assessed, for example, using
Patient Global Impression of Severity (PGI-S),
a Physician Global Assessment (PGA),
a change in titer of antinuclear antibody (ANA),
a change in titer of Anti-double stranded DNA (dsDNA) antibodies,
a change in complement C3,
a change in complement C4, and/or
SLE Disease Activity Index 2000 (SLEDAI-2K).

The scoring for SLEDAI-2K is described in Gladman et al., The Journal of Rheumatology. 29(2):288-291. In some embodiments, a response to treatment for SLE is a renal response, e.g., a complete renal response or a partial renal response. In some embodiments, a renal response can be quantified by a decrease in urine protein creatinine ratio (UPCR) or an increase in estimated glomerular filtration rate (eGFR; e.g., as determined according to the Chronic Kidney Disease Epidemiology Collaboration (CKD-EPI) equation).

A “Patient Global Impression of Severity” or “PGI-S” refers to a single-item question that asks the patient how they would rate their overall lupus severity over the past week. The form asks, “[p]lease choose the response below that best describes the severity of your Lupus over the past week.” There are five categories of response: (1) none, (2) mild, (3) moderate, (4) severe, and (5) very severe.

“Physician Global Assessment” or “PGA” refers to an evaluation instrument to be used by a physician when assessing a patient regarding the patient's systemic lupus erythematosus. The form asks, “Please answer the following questions by placing a vertical mark though the line (the “line” depicts a subdivided scale from None (0) to 3 (Severe). The question asked is, “On the line below, where would you rate the subject's SLE over the past 28 days?”

“Antinuclear antibodies” (ANAs) refer to a diverse group of autoantibodies that recognize nuclear macromolecules and their complexes. ANAs represent key biomarkers in the evaluation of rheumatic diseases, such as systemic lupus erythematosus.

“Anti-double stranded DNA (dsDNA) antibodies” means a group of autoantibodies that recognize double stranded DNA (dsDNA).

An “effective response of a subject,” an “effective response of an individual,” or a patient's, a subject's, or an individual's “responsiveness” to treatment with a medicament and similar wording refers to the clinical or therapeutic benefit imparted to a patient as risk for, or suffering from, a disease or disorder, such as systemic lupus erythematosus. In one embodiment, such benefit includes reducing or eliminating any symptom of systemic lupus erythematosus or a change in a biomarker associated with systemic lupus erythematosus that trends to, or achieves, a healthy value.

A patient who “does not have an effective response to treatment” refers to a patient who does not have improving signs or symptoms of systemic lupus erythematosus.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the patient antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

“Anti-Smith antibody” or “anti-Sm antibody” refers to an antibody that specifically binds the Sm antigen. Anti-Smith antibodies are autoantibodies specific to SLE. In some embodiments, the upper limit of normal (ULN) for an anti-Smith antibody is <7 units (U)/mL. The ULN may vary depending on the methods of detection and/or test site. In other embodiments, the ULN for an anti-Smith antibody is <10 units (U)/mL.

“Anti-double stranded DNA antibody” or “anti-dsDNA antibody” refers to an antibody that specifically binds double-stranded DNA. Anti-dsDNA antibodies are autoantibodies specific to SLE. In some embodiments, the upper limit of normal (ULN) for an anti-double stranded DNA antibody is <25 international units (IU)/mL. The ULN may vary depending on the methods of detection and/or test site. In other embodiments, the ULN for an anti-dsDNA antibody is <100 international units (IU)/mL.

An “autoantibody” is an antibody that targets an individual's own proteins, cells, and tissue. Some autoimmune disorders, e.g., SLE, are associated with autoantibodies. In particular, autoantibodies associated with SLE include anti-Sjögren's-syndrome-related antigen A (anti-SSA), anti-double-stranded-DNA (anti-dsDNA), anti-Smith, anti-histone, and anti-thrombin autoantibodies.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (K_{D}) of ≤1 μM, ≤100 nM, ≤10 nM, ≤1 nM, ≤0.1 nM, s 0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD20 antibody binds to an epitope of CD20 that is conserved among CD20 from different species.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI RefSeq Nos. NP_068769.2 and NP_690605.1), which is 297 amino acids in length and may be generated, for example, from variant mRNA transcripts that lack a portion of the 5′ UTR (see, e.g., NCBI RefSeq No. NM_021950.3) or longer variant mRNA transcripts (see, e.g., NCBI RefSeq No. NM_152866.2).

“Anti-CD20 monoclonal antibody therapy” refers to the administering of an anti-CD20 monoclonal antibody to a patient. Examples include obinutuzumab, rituximab, ocrelizumab, and ofatumumab.

The terms “anti-CD20/anti-CD3 bispecific antibody,” “bispecific anti-CD20/anti-CD3 antibody,” and “antibody that binds to CD20 and CD3,” or variants thereof, refer to a multispecific antibody (e.g., a bispecific antibody) that is capable of binding to CD20 and CD3 with sufficient affinity such that the antibody is useful as a diagnostic and/or therapeutic agent in targeting CD20 and/or CD3. In one embodiment, the extent of binding of a bispecific antibody that binds to CD20 and CD3 to an unrelated, non-CD3 protein and/or non-CD20 protein is less than about 10% of the binding of the antibody to CD3 and/or CD20 as measured, e.g., by a radioimmunoassay (RIA). In certain embodiments, a bispecific antibody that binds to CD20 and CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, a bispecific antibody that binds to CD20 and CD3 binds to an epitope of CD3 that is conserved among CD3 from different species and/or an epitope of CD20 that is conserved among CD20 from different species. In one embodiment, the bispecific antibody binds monovalently to CD20 and binds monovalently to CD3. In one embodiment, a bispecific antibody that binds to CD20 and CD3 is mosunetuzumab.

As used herein, the term “mosunetuzumab” refers to an anti-CD20/anti-CD3 bispecific antibody having the International Nonproprietary Names for Pharmaceutical Substances (INN) List 117 (WHO Drug Information, Vol. 31, No. 2, 2017, p. 303-305), or the CAS Registry Number 1905409-39-3.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater Affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, Affinity and K_{D} values are inversely related. A high Affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

An “effective amount” of an agent, e.g., a pharmaceutical composition, refers to an amount of the antibody or medicament effective, at dosages and for periods of time necessary, to achieve the desired treatment as defined above.

A “pharmaceutical formulation” or “pharmaceutical composition” refers to a preparation which is in such form as to permit the biological activity of an active ingredient contained therein to be effective, and which contains no additional components which are unacceptably toxic to a patient to which the formulation would be administered. In some instances, mosunetuzumab may be provided in a pharmaceutical composition as disclosed in U.S. Pat. No. 11,958,906. In some instances, mosunetuzumab is provided as a pharmaceutical composition comprising 1 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some instances, mosunetuzumab is provided as a pharmaceutical composition comprising 10 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some instances, mosunetuzumab is provided as a pharmaceutical composition comprising 45 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some instances, the pharmaceutical composition further comprises histidine acetate.

A “pharmaceutically acceptable carrier” refers to an ingredient in a pharmaceutical formulation, other than an active ingredient, which is nontoxic to a patient. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

The term “renal impairment” refers to impairment in the function of the kidney, e.g., chronic kidney disease (CKD). In some embodiments, the level of renal impairment or CKD is quantified using estimated glomerular filtration rate (eGFR; e.g., as determined according to the Chronic Kidney Disease Epidemiology Collaboration (CKD-EPI) equation). In some embodiments, “significant renal impairment” refers to eGFR <30 mL/min/1.73 m².

The term “remission” refers to a cSLEDAI score of 0 maintained on stable SLE treatment for ≥6 months by 1 year.

The term “clinical remission” refers to maintaining a SLEDAI score (SLEDAI-2K or cSLEDAI) of 0 for at least 6 months without being administered any SLE treatment.

The term “clinical response” refers to having all of the following for at least 6 months: a cSLEDAI score of 0 after completion of mosunetuzumab treatment, stable dosing of SLE therapies (e.g., without any changes in dosing of the SLE therapies) for at least 6 months, and prednisone (or equivalent) dose 55 mg/day. In some embodiments, a clinical response refers to achieving Definition of Remission in SLE (DORIS; as defined in van Vollenhoven et al., Lupus Sci Med., 9(1):e000538corr1, 2022).

The term “complete renal response” refers to having all of the following: a UPCR <0.5 g/g, eGFR ≥85% of baseline as calculated using the CKD-EPI equation or ≥60 ml/min per 1.73 m² of body-surface area.

The term “partial renal response” refers to having all of the following: ≥50% reduction in UPCR from baseline, UPCR <1 g/g (or <3 g/g if the baseline UPCR was ≥3 g/g), and eGFR ≥85% of baseline as calculated using the CKD-EPI equation.

The term “Background Immunosuppressant Discontinuation Eligibility Point” or “BDEP” refers to the time point at which an individual or subject may discontinue standard background immunosuppressant therapy based on said individual's or subject's disease activity. Once an individual or subject reaches the BDEP, other immunosuppressant therapies (e.g., other than mosunetuzumab or additional therapeutic agents used in the methods described herein), specifically MMF and AZA, are discontinued without tapering. The decision to taper and discontinue immunosuppression is based on what is safe and clinically appropriate for each individual or subject.

The term “background immunotherapy” or “background immunosuppressant therapy” refers to standard background immunosuppressant therapy for treating SLE, e.g., administered to an Invidia with a particular disease (e.g., SLE) activity. In some embodiments, after an individual has been treated according to any of the methods described herein, the individual may discontinue administration of background immunotherapy. In some embodiments, background immunotherapy (e.g., that is to be discontinued) includes mycophenolate mofetil, azathioprine, and/or methotrexate. In some embodiments, the background immunotherapy includes mycophenolate mofetil. In some embodiments, the background immunotherapy includes azathioprine. In some embodiments, the background immunotherapy includes methotrexate. In some embodiments, the background immunotherapy (e.g., that is to be discontinued) does not include hydroxychloroquine. In some embodiments, the background immunotherapy includes oral corticosteroids, e.g., oral prednisone or equivalent. In some embodiments, the background immunotherapy includes calcineurin inhibitors.

### III. Therapeutic Methods

Provided herein are methods of treating an individual having a systemic lupus erythematosus (SLE) by administration of mosunetuzumab. In particular, the present invention relates to methods of treating an individual having an SLE by subcutaneous administration of mosunetuzumab. In some embodiments, the individual is subcutaneously administered mosunetuzurnab as a monotherapy. In some embodiments, the individual has active Class III or IV lupus nephritis (LN), as defined by the 2018 International Society of Nephrology/Renal Pathology Society (ISN/RPS) classification guidelines (Bajema et al., Kidney International. 93(4):389-796, 2018). In some embodiments, the Class III or IV LN was determined using a renal biopsy sample. In some embodiments, the renal biopsy sample was determined to have ≥3 points on the NIH activity index, excluding any contribution from interstitial activity; and <50% glomerulosclerosis and <50% tubulointerstitial fibrosis.

In some embodiments, the individual has: (a) UPCR ≥1.15 g/g; (b) Complement C3 below the lower limit of normal (e.g., below 90 mg/dL, e.g., in a patient over 17 years old); and/or (c) active LN that has been treated for at least 6 months with standard nonsteroidal LN therapy prior to administration of mosunetuzumab. In some embodiments, the individual has Complement C4 below the lower limit of normal (e.g., below 12 mg/dL, e.g., in a patient over 17 years old). In some embodiments, the individual is positive (e.g., >75 IU/mL), negative (e.g., <30 IU/mL) or borderline (e.g., 30-75 IU/mL) for anti-double-stranded DNA antibody. In some embodiments, the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitor is baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept. In some embodiments, the individual does not have LN. In some embodiments, the individual has SLE diagnosed at least 6 months prior to screening per 2019 European League Against Rheumatism (EULAR)/American College of Rheumatology (ACR) SLE Classification Criteria (Aringer et al., Arthritis Rheumatol, 71(9):1400-1412, 2019).

### A. Therapeutic Methods for Dosing of Mosunetuzumab

In one aspect, the invention features a method of treating an individual having systemic lupus erythematosus (SLE), comprising subcutaneously administering to the individual mosunetuzumab according to a dosing regimen comprising at least a first dosing cycle and a second dosing cycle, wherein: (a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg); and (b) the second dosing cycle comprises a single dose (C2D1) of mosunetuzumab, wherein the C2D1 of mosunetuzumab is about 45 mg (e.g., 45 mg±10%; 45 mg±4.5 mg).

In some embodiments, the first dosing cycle is a 21-day dosing cycle. In some embodiments, the C1D1, C1D2, and C1D3 are administered on or about Days 1, 8, and 15, respectively, of the first dosing cycle. In some embodiments, the second dosing cycle is a 21-day dosing cycle. In some embodiments, the C2D1 is administered on or about Day 1 of the second dosing cycle.

In some embodiments, the method further comprises one or more additional dosing cycles. In some embodiments, the method comprises one to seven (e.g., one, two, three, four, five, six, or seven) additional dosing cycles. In some embodiments, the method comprises seven additional dosing cycles. In some embodiments, each additional dosing cycle is a 21-day dosing cycle. In some embodiments, one or more of the additional dosing cycles comprises an additional single dose of mosunetuzumab. In some embodiments, the additional single dose of mosunetuzumab is administered on Day 1 of the respective additional dosing cycle. In some embodiments, the additional single dose of mosunetuzumab is about 45 mg (e.g., 45 mg±10%; 45 mg±4.5 mg).

In one aspect, the invention features a method of treating an individual having SLE, comprising subcutaneously administering to the individual mosunetuzumab according to a dosing regimen comprising nine 21-day dosing cycles, wherein: (a) the first dosing cycle comprises a first dose (C1D1), a second dose (C1D2), and a third dose (C1D3), wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg), and wherein the C1D1, the C1D2, and the C1D3 are administered on Days 1, 8, and 15, respectively, of the first dosing cycle; and (b) the second to ninth dosing cycles each comprises a single dose (C2D1-C9D1) of mosunetuzumab administered on Day 1 of each respective dosing cycle, wherein each of the C2D1-C9D1 is about 45 mg (e.g., 45 mg±10%; 45 mg±4.5 mg).

In some embodiments, mosunetuzumab is administered as a monotherapy.

In some embodiments, prior to administration of any dose of mosunetuzumab, the individual has been previously administered an oral corticosteroid, an antimalarial agent, or an immunosuppressant. In some embodiments, the individual has been administered an antimalarial agent at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the antimalarial agent is hydroxychloroquine, chloroquine, or quinacrine. In some embodiments, the individual has been administered an immunosuppressant at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the immunosuppressant is azathioprine, mycophenolate mofetil, mycophenolic acid, or methotrexate.

In some embodiments, the individual does not have a lupus-associated neuropsychiatric disease. In some embodiments, the lupus-associated neuropsychiatric disease is meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, or mononeuritis multiplex.

In some embodiments, the individual does not have active overlap syndrome with mixed connective tissue disease or systemic sclerosis within a year prior to being administered mosunetuzumab. In some embodiments, the individual does not have catastrophic or severe antiphospholipid syndrome within a year prior to being administered mosunetuzumab, unless the severe antiphospholipid syndrome has been adequately controlled by administering anticoagulant therapy to the individual at least 2 months (e.g., 2, 3, 4, 5, 6, 7, 8, 9, 10, 11, 12, 18, 24, 30, 36 months, or more) prior to being administered mosunetuzumab.

In some embodiments, the individual has not been administered: (a) at least 12 months (e.g., 12, 18, 24, 30, 36, 42, 48 months, or more) prior to being administered mosunetuzumab: (i) an anti-CD19 antibody therapy, or (ii) an anti-CD20 monoclonal antibody therapy; (b) at least 30 days (e.g., 30, 60, 90, 120, 150, 180 days or more) prior to being administered mosunetuzumab: (i) kinase inhibitors of Janus Kinase (JAK) kinase, Bruton tyrosine kinase, or tyrosine kinase 2, or (ii) a calcineurin inhibitor; or (c) at least 2 months (e.g., 2, 3, 4, 5, 6, 7, 8, 9, 10, 11, 12, 18, 24, 30, 36 months, or more) prior to being administered mosunetuzumab: (i) cyclophosphamide, or (ii) a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitors are baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, the individual does not have significant lupus-associated renal disease or significant renal impairment.

In some embodiments, the individual does not have any of the following laboratory parameters: (a) total bilirubin >1.5×upper limit of normal (ULN); (b) absolute neutrophil count (ANC)<1.5×10⁹/L (<1500/μL); (c) platelet count <75×10⁹/L (100,000/mm³); (d) hemoglobin <90 g/L; (e) estimated glomerular filtration rate (eGFR)<30 ml/min/1.73 m² calculated according to the Chronic Kidney Disease Epidemiology Collaboration (CKD-EPI) equation; or (f) aspartate transferase (AST) or alanine transaminase (ALT) >2.5×ULN.

In some embodiments, the individual achieves an evaluable response after completion of the dosing regimen. In some embodiments, the evaluable response is remission, a clinical response, a complete renal response (CRR), or a partial renal response. In some embodiments, the evaluable response is remission, and wherein the individual: (a) maintains a clinical SLE Disease Activity Index=0 for 6 months after administration of the final dose of mosunetuzumab; and (b) is not administered any additional therapeutic agents for treating SLE, except for an antimalarial agent, for 6 months after administration of the final dose of mosunetuzumab. In some embodiments, the antimalarial agent is hydroxychloroquine, chloroquine, or quinacrine. In some embodiments, the individual is not administered any additional therapeutic agents for treating SLE, for 6 months after administration of the final dose of mosunetuzumab. In some embodiments, the evaluable response is a clinical response, and wherein the individual: (a) maintains a clinical SLE Disease Activity Index (cSLEDAI)=0 for 6 months after administration of the final dose of mosunetuzumab; and (b) is administered a stable dosing of SLE therapy for 6 months after administration of the final dose of mosunetuzumab; and (c) is administered ≤5 mg/day prednisone or equivalent for 6 months after administration of the final dose of mosunetuzumab. In some embodiments, the evaluable response is a complete renal response, wherein the individual has: (a) urine protein creatinine ratio (UPCR)<0.5 g/g; and (b) eGFR ≥85% of baseline or ≥60 ml/min/1.73 m² calculated according to the CKD-EPI equation. In some embodiments, the evaluable response is a partial renal response, wherein the individual has: (a) ≥50% reduction in UPCR from baseline; (b) UPCR <1 g/g, or UPCR <3 g/g if baseline UPCR was ≥3 g/g; and (c) eGFR ≥85% of baseline calculated according to the CKD-EPI equation.

In some embodiments, the individual exhibits at least one reduced symptom of SLE as measured using a Patient Global Impression of Severity (PGI-S), a Physician Global Assessment (PGA), a decrease in titer of antinuclear antibody (ANA), a decrease in titer of anti-double stranded DNA (dsDNA) antibody (IgG) titer, an increase in complement C3 levels, or an increase in complement C4 levels after completion of the dosing regimen. In some embodiments, the at least one reduced symptom is a change in response of at least one step on the PGI-S from a previous response, where the change is one change of from “very severe” to “severe,” “severe” to “moderate,” “moderate” to “mild,” or “mild” to “none.” In some embodiments, the at least one reduced symptom is a change in rating by a healthcare provider or other appropriately trained professional using the PGA, where the change is a decrease from a previous rating using the PGA. In some embodiments, the individual exhibits a decrease in titer of anti-nuclear antibody (ANA). In some embodiments, the individual exhibits a decrease in titer of anti-dsDNA antibody (IgG) titer. In some embodiments, the individual exhibits an increase in complement C3 levels. In some embodiments, the individual exhibits an increase in complement C4 levels.

In some embodiments, any of the methods herein above further comprises administering to the individual a corticosteroid or a calcineurin inhibitor. In some embodiments, (a) the corticosteroid comprises hydrocortisone, cortisone acetate, prednisolone, dexamethasone, betamethasone, triamcinolone, beclomethasone, methylprednisolone or prednisone; or (b) the calcineurin inhibitor comprises ciclosporin, tacrolimus, or vocolosporin.

In some embodiments, the individual has active Class III or IV lupus nephritis (LN), as defined by the 2018 International Society of Nephrology/Renal Pathology Society (ISN/RPS) classification guidelines (Bajema et al., Kidney International. 93(4):389-796, 2018). In some embodiments, the Class III or IV LN was determined using a renal biopsy sample. In some embodiments, the renal biopsy sample was determined to have ≥3 points on the NIH activity index, excluding any contribution from interstitial activity; and <50% glomerulosclerosis and <50% tubulointerstitial fibrosis.

In some embodiments, the individual has: (a) UPCR ≥1.15 g/g; (b) Complement C3 below the lower limit of normal (e.g., below 90 mg/dL, e.g., in a patient over 17 years old); and/or (c) active LN that has been treated for at least 6 months with standard nonsteroidal LN therapy prior to administration of mosunetuzumab. In some embodiments, the individual has Complement C4 below the lower limit of normal (e.g., below 12 mg/dL, e.g., in a patient over 17 years old). In some embodiments, the individual is positive (e.g., >75 IU/mL), negative (e.g., <30 IU/mL) or borderline (e.g., 30-75 IU/mL) for anti-double-stranded DNA antibody. In some embodiments, the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitor is baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, the individual does not have LN.

In some embodiments, the individual has autoantibody positivity, hypocomplementemia, and high disease activity despite treatment with advanced therapy. In some embodiments, the individual has an anti-double-stranded DNA antibody or anti-Smith antibody above ULN; or a Complement C3 level below the lower limit of normal. In some embodiments, the individual has a Complement C4 level below the normal limit of normal. In some embodiments, the individual is positive (e.g., ≥75 IU/mL), negative (e.g., <30 IU/mL) or borderline (e.g., 30-75 IU/mL) for anti-double-stranded DNA antibody. In some embodiments, the individual has an Extrarenal SLE Disease Activity Index 2000 (SLEDAI-2K) score ≥8, excluding points for: (a) proteinuria, pyuria, hematuria, casts, alopecia, fever, and headache; (b) rash if Cutaneous LE Disease Area and Severity Index (CLASI; Albrecht et al., J Invest Dermatol. 125(5):889-894, 2005) activity score is <6; (c) mucosal ulcers that do not significantly interfere with oral intake; and (d) arthritis with <4 joints that are both swollen and tender or with <2 located in the wrist or hand. In some embodiments, the advanced therapy comprises treatment for at least 6 months with cyclophosphamide, belimumab, ustekinumab, anifrolumab, secukinumab, atacicept, obinutuzumab, rituximab, ocrelizumab, or ofatumumab.

In one aspect, the invention features a method of treating a population of individuals having SLE, comprising subcutaneously administering to each individual of the population mosunetuzumab according to a dosing regimen comprising at least a first dosing cycle and a second dosing cycle, wherein: (a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg); and (b) the second dosing cycle comprises a single dose (C2D1) of mosunetuzumab, wherein the C2D1 of mosunetuzumab is about 45 mg (e.g., 45 mg±10%; 45 mg±4.5 mg).

In some embodiments, the first dosing cycle is a 21-day dosing cycle. In some embodiments, the C1D1, C1D2, and C1D3 are administered on or about Days 1, 8, and 15, respectively, of the first dosing cycle. In some embodiments, the second dosing cycle is a 21-day dosing cycle. In some embodiments, the C2D1 is administered on or about Day 1 of the second dosing cycle.

In some embodiments, the method further comprises one or more additional dosing cycles. In some embodiments, the method comprises one to seven (e.g., one, two, three, four, five, six, or seven) additional dosing cycles. In some embodiments, the method comprises seven additional dosing cycles. In some embodiments, each additional dosing cycle is a 21-day dosing cycle. In some embodiments, one or more of the additional dosing cycles comprises an additional single dose of mosunetuzumab. In some embodiments, the additional single dose of mosunetuzumab is administered on Day 1 of the respective additional dosing cycle. In some embodiments, the additional single dose of mosunetuzumab is about 45 mg (e.g., 45 mg±10%; 45 mg±4.5 mg).

In one aspect, the invention features a method of treating a population of individuals having SLE, comprising subcutaneously administering to each individual of the population mosunetuzumab according to a dosing regimen comprising nine 21-day dosing cycles, wherein: (a) the first dosing cycle comprises a first dose (C1D1), a second dose (C1D2), and a third dose (C1D3), wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg), and wherein the C1D1, the C1D2, and the C1D3 are administered on Days 1, 8, and 15, respectively, of the first dosing cycle; and (b) the second to ninth dosing cycles each comprises a single dose (C2D1-C9D1) of mosunetuzumab administered on Day 1 of each respective dosing cycle, wherein each of the C2D1-C9D1 is about 45 mg (e.g., 45 mg±10%; 45 mg±4.5 mg).

In some embodiments, mosunetuzumab is administered as a monotherapy.

In some embodiments, prior to administration of any dose of mosunetuzumab, an individual of the population has been previously administered an oral corticosteroid, an antimalarial agent, or an immunosuppressant. In some embodiments, an individual of the population has been administered an antimalarial agent at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the antimalarial agent is hydroxychloroquine, chloroquine, or quinacrine. In some embodiments, an individual of the population has been administered an immunosuppressant at a stable dose for at least 4 weeks (e.g., 4, 5, 6, 7, 8, 9, 10, 11, 12, 16, 20, 24, 28, 32 weeks, or more) prior to being administered mosunetuzumab. In some embodiments, the immunosuppressant is azathioprine, mycophenolate mofetil, mycophenolic acid, or methotrexate.

In some embodiments, an individual of the population does not have a lupus-associated neuropsychiatric disease. In some embodiments, the lupus-associated neuropsychiatric disease is meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, or mononeuritis multiplex.

In some embodiments, an individual of the population does not have active overlap syndrome with mixed connective tissue disease or systemic sclerosis within a year prior to being administered mosunetuzumab. In some embodiments, an individual of the population does not have catastrophic or severe antiphospholipid syndrome within a year prior to being administered mosunetuzumab, unless the severe antiphospholipid syndrome has been adequately controlled by administering anticoagulant therapy to the individual at least 2 months (e.g., 2, 3, 4, 5, 6, 7, 8, 9, 10, 11, 12, 18, 24, 30, 36 months, or more) prior to being administered mosunetuzumab.

In some embodiments, an individual of the population has not been administered: (a) at least 12 months (e.g., 12, 18, 24, 30, 36, 42, 48 months, or more) prior to being administered mosunetuzumab: (i) an anti-CD19 antibody therapy, or (ii) an anti-CD20 monoclonal antibody therapy; (b) at least 30 days (e.g., 30, 60, 90, 120, 150, 180 days or more) prior to being administered mosunetuzumab: (i) kinase inhibitors of Janus Kinase (JAK) kinase, Bruton tyrosine kinase, or tyrosine kinase 2, or (ii) a calcineurin inhibitor; or (c) at least 2 months (e.g., 2, 3, 4, 5, 6, 7, 8, 9, 10, 11, 12, 18, 24, 30, 36 months, or more) prior to being administered mosunetuzumab: (i) cyclophosphamide, or (ii) a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitors are baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, an individual of the population does not have significant lupus-associated renal disease or significant renal impairment.

In some embodiments, an individual of the population does not have any of the following laboratory parameters: (a) total bilirubin >1.5×upper limit of normal (ULN); (b) absolute neutrophil count (ANC)<1.5×10⁹/L (<1500/μL); (c) platelet count <75×10⁹/L (100,000/mm³); (d) hemoglobin <90 g/L; (e) estimated glomerular filtration rate (eGFR)<30 ml/min/1.73 m² calculated according to the Chronic Kidney Disease Epidemiology Collaboration (CKD-EPI) equation; or (f) aspartate transferase (AST) or alanine transaminase (ALT) >2.5×ULN.

In some embodiments, an individual of the population achieves an evaluable response after completion of the dosing regimen. In some embodiments, the evaluable response is remission, a clinical response, a complete renal response (CRR), or a partial renal response. In some embodiments, the evaluable response is remission, and wherein an individual of the population: (a) maintains a clinical SLE Disease Activity Index=0 for 6 months after administration of the final dose of mosunetuzumab; and (b) is not administered any additional therapeutic agents for treating SLE, except for an antimalarial agent, for 6 months after administration of the final dose of mosunetuzumab. In some embodiments, the antimalarial agent is hydroxychloroquine, chloroquine, or quinacrine. In some embodiments, an individual of the population is not administered any additional therapeutic agents for treating SLE, for 6 months after administration of the final dose of mosunetuzumab. In some embodiments, the evaluable response is a clinical response, and wherein an individual of the population: (a) maintains a clinical SLE Disease Activity Index (cSLEDAI)=0 for 6 months after administration of the final dose of mosunetuzumab; and (b) is administered a stable dosing of SLE therapy for 6 months after administration of the final dose of mosunetuzumab; and (c) is administered ≤5 mg/day prednisone or equivalent for 6 months after administration of the final dose of mosunetuzumab. In some embodiments, the evaluable response is a complete renal response, wherein an individual of the population has: (a) urine protein creatinine ratio (UPCR)<0.5 g/g; and (b) eGFR ≥85% of baseline or ≥60 ml/min/1.73 m² calculated according to the CKD-EPI equation. In some embodiments, the evaluable response is a partial renal response, wherein an individual of the population has: (a) ≥50% reduction in UPCR from baseline; (b) UPCR <1 g/g, or UPCR <3 g/g if baseline UPCR was ≥3 g/g; and (c) eGFR ≥85% of baseline calculated according to the CKD-EPI equation.

In some embodiments, an individual of the population exhibits at least one reduced symptom of SLE as measured using a Patient Global Impression of Severity (PGI-S), a Physician Global Assessment (PGA), a decrease in titer of antinuclear antibody (ANA), a decrease in titer of anti-double stranded DNA (dsDNA) antibody (IgG) titer, an increase in complement C3 levels, or an increase in complement C4 levels after completion of the dosing regimen. In some embodiments, the at least one reduced symptom is a change in response of at least one step on the PGI-S from a previous response, where the change is one change of from “very severe” to “severe,” “severe” to “moderate,” “moderate” to “mild,” or “mild” to “none.” In some embodiments, the at least one reduced symptom is a change in rating by a healthcare provider or other appropriately trained professional using the PGA, where the change is a decrease from a previous rating using the PGA. In some embodiments, an individual of the population exhibits a decrease in titer of anti-nuclear antibody (ANA). In some embodiments, an individual of the population exhibits a decrease in titer of anti-dsDNA antibody (IgG) titer. In some embodiments, an individual of the population exhibits an increase in complement C3 levels. In some embodiments, an individual of the population exhibits an increase in complement C4 levels.

In some embodiments, an individual of the population is further administered tocilizumab if the individual experiences cytokine release syndrome (CRS).

In some embodiments, any of the methods herein above further comprises administering to an individual of the population a corticosteroid or a calcineurin inhibitor. In some embodiments, (a) the corticosteroid comprises hydrocortisone, cortisone acetate, prednisolone, dexamethasone, betamethasone, triamcinolone, beclomethasone, methylprednisolone or prednisone; or (b) the calcineurin inhibitor comprises ciclosporin, tacrolimus, or vocolosporin.

In some embodiments, an individual of the population has active Class III or IV lupus nephritis (LN), as defined by the 2018 International Society of Nephrology/Renal Pathology Society (ISN/RPS) classification guidelines (Bajema et al., Kidney International. 93(4):389-796, 2018). In some embodiments, the Class III or IV LN was determined using a renal biopsy sample. In some embodiments, the renal biopsy sample was determined to have ≥3 points on the NIH activity index, excluding any contribution from interstitial activity; and <50% glomerulosclerosis and <50% tubulointerstitial fibrosis.

In some embodiments, an individual of the population has: (a) UPCR ≥1.15 g/g; (b) Complement C3 below the lower limit of normal (e.g., below 90 mg/dL, e.g., in a patient over 17 years old); and/or (c) active LN that has been treated for at least 6 months with standard nonsteroidal LN therapy prior to administration of mosunetuzumab. In some embodiments, the individual has Complement C4 below the lower limit of normal (e.g., below 12 mg/dL, e.g., in a patient over 17 years old). In some embodiments, the individual is positive (e.g., >75 IU/mL), negative (e.g., <30 IU/mL) or borderline (e.g., 30-75 IU/mL) for anti-double-stranded DNA antibody. In some embodiments, the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy. In some embodiments, (a) the anti-CD19 therapy is blinatumomab; (b) the anti-CD20 therapy is obinutuzumab, rituximab, ocrelizumab, or ofatumumab; (c) the kinase inhibitor is baricitinib, tofacitinib, upadacitinib, filgotinib, ibrutinib, or fenebrutinib; (d) the calcineurin inhibitor is tacrolimus, ciclosporin, or voclosporin; or (e) the biologic therapy is belimumab, ustekinumab, anifrolumab, secukinumab, or atacicept.

In some embodiments, an individual of the population does not have LN.

In some embodiments, an individual of the population has autoantibody positivity, hypocomplementemia, and high disease activity despite treatment with advanced therapy. In some embodiments, an individual of the population has an anti-double-stranded DNA antibody or anti-Smith antibody above ULN; or a Complement C3 level below the lower limit of normal. In some embodiments, an individual of the population has an Extrarenal SLE Disease Activity Index 2000 (SLEDAI-2K) score ≥8, excluding points for: (a) proteinuria, pyuria, hematuria, casts, alopecia, fever, and headache; (b) rash if Cutaneous LE Disease Area and Severity Index (CLASI; Albrecht et al., J Invest Dermatol. 125(5):889-894, 2005) activity score is <6; (c) mucosal ulcers that do not significantly interfere with oral intake; and (d) arthritis with <4 joints that are both swollen and tender or with <2 located in the wrist or hand. In some embodiments, the advanced therapy comprises treatment for at least 6 months with cyclophosphamide, belimumab, ustekinumab, anifrolumab, secukinumab, atacicept, obinutuzumab, rituximab, ocrelizumab, or ofatumumab.

In some embodiments, an individual of the population is administered dexamethasone, diphenhydramine, and/or acetaminophen/paracetamol prior to administration of any dose of mosunetuzumab. In some embodiments, an individual of the population is administered dexamethasone, diphenhydramine, and acetaminophen/paracetamol prior to administration of the C1D1, C1D2, C1D3, and C2D1 of mosunetuzumab. In some embodiments, dexamethasone is administered orally at a dose of about 20 mg (e.g., 20 mg±10%; 20 mg±2 mg); diphenhydramine is administered orally at a dose of about 25 mg to about 50 mg (e.g., 25 mg to 45 mg, 25 mg to 40 mg, 25 mg to 35 mg, 25 mg to 30 mg, 30 mg to 50 mg, 40 mg to 50 mg, 45 mg to 50 mg, 30 mg to 35 mg, 30 mg to 45 mg, 35 mg to 45 mg, 40 mg to 45 mg, or 30 mg to 40 mg; e.g., about 25 mg, about 30 mg, about 35 mg, about 40 mg, about 45 mg, or about 50 mg); and acetaminophen/paracetamol is administered orally at a dose of about 650 mg to about 1000 mg (e.g., 650 mg to 900 mg, 650 mg to 80 mg, 650 mg to 700 mg, 700 mg to 1000 mg, 800 mg to 1000 mg, 900 mg to 1000 mg, 700 mg to 800 mg, 800 mg to 900 mg, 700 mg to 900 mg; e.g., about 650 mg, about 700 mg, about 750 mg, about 800 mg, about 850 mg, about 900 mg, about 950 mg, or about 1000 mg).

In some embodiments, administering one or more doses of mosunetuzumab to the individual or individuals induces a B cell depletion in the individual or individuals. In some embodiments, the B cell depletion is a deep-tissue B cell depletion and/or a peripheral B cell depletion. In some embodiments, the deep-tissue B cell depletion is determined by tissue biopsy. In some embodiments, the deep-tissue B cell depletion is determined by kidney biopsy. In some embodiments, the peripheral B cell depletion is determined by blood flow cytometry. In some embodiments, the amount of B cell depletion is determined relative to a reference level of B cells. In some embodiments, the reference level of B cells is determined at a time prior to administration of any dose of mosunetuzumab.

In one aspect, the invention features a method of inducing B cell depletion in an individual, comprising subcutaneously administering to the individual mosunetuzumab according to a dosing regimen comprising nine 21-day dosing cycles, wherein: (a) the first dosing cycle comprises a first dose (C1D1), a second dose (C1D2), and a third dose (C1D3), wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg), and wherein the C1D1, the C1D2, and the C1D3 are administered on Days 1, 8, and 15, respectively, of the first dosing cycle; and (b) the second to ninth dosing cycles each comprises a single dose (C2D1-C9D1) of mosunetuzumab administered on Day 1 of each respective dosing cycle, wherein each of the C2D1-C9D1 is about 45 mg (e.g., 45 mg±10%; 45 mg±4.5 mg).

In some embodiments, the B cell depletion is a deep-tissue B cell depletion and/or a peripheral B cell depletion. In some embodiments, the deep-tissue B cell depletion is determined by tissue biopsy. In some embodiments, the deep-tissue B cell depletion is determined by kidney biopsy. In some embodiments, the peripheral B cell depletion is determined by blood flow cytometry. In some embodiments, the amount of B cell depletion is determined relative to a reference level of B cells. In some embodiments, the reference level of B cells is determined at a time prior to administration of any dose of mosunetuzumab. In some embodiments, the individual has SLE.

In some embodiments, an individual or individuals treated according to any of the methods described herein exhibits a B cell depletion. In some embodiments, the B cell depletion is a deep-tissue B cell depletion and/or a peripheral B cell depletion. In some embodiments, the deep-tissue B cell depletion is determined by tissue biopsy. In some embodiments, the deep-tissue B cell depletion is determined by kidney biopsy. In some embodiments, the peripheral B cell depletion is determined by blood flow cytometry. In some embodiments, the amount of B cell depletion is determined relative to a reference level of B cells. In some embodiments, the reference level of B cells is determined at a time prior to treatment according to any of the methods described herein. In some embodiments, the reference level of B cells is determined at a time prior to administration of any dose of mosunetuzumab.

In some embodiments, after an individual is treated according to any of the methods described herein, the amount of any additional therapeutic agents for treating SLE administered to the individual is tapered. In some embodiments, the amount of any additional therapeutic agents for treating SLE administered to the individual is tapered after treatment according to any of the methods described herein. In some embodiments, the amount of any additional therapeutic agents for treating SLE administered to the individual is tapered after administration of any dose of mosunetuzumab. In some embodiments, the amount of any additional therapeutic agents for treating SLE administered to the individual is tapered after the individual exhibits a B cell depletion. In some embodiments, the amount of any additional therapeutic agents for treating SLE administered to the individual is tapered after the individual exhibits a deep-tissue B cell depletion and/or a peripheral B cell depletion. In some embodiments, the amount of any additional therapeutic agents for treating SLE administered to the individual is tapered after the individual achieves a cSLEDAI=0.

In some embodiments, any of the methods described herein further comprises discontinuing administering a background immunotherapy to the individual. In some embodiments, any of the methods described herein further comprises discontinuing administering a background immunotherapy to the individual after treatment according to any of the methods described herein. In some embodiments, any of the methods described herein further comprises discontinuing administering a background immunotherapy to the individual after administration of any dose of mosunetuzumab to the individual. In some embodiments, any of the methods described herein further comprises discontinuing administering a background immunotherapy to the individual after the individual exhibits a B cell depletion. In some embodiments, any of the methods described herein further comprises discontinuing administering a background immunotherapy to the individual after the individual exhibits a deep-tissue B cell depletion and/or a peripheral B cell depletion. In some embodiments, any of the methods described herein further comprises discontinuing administering a background immunotherapy to the individual after the individual achieves a cSLEDAI=0. In some embodiments, the background immunotherapy comprises at least one selected from the group consisting of mycophenolate mofetil, azathioprine, and methotrexate. In some embodiments, the background immunotherapy comprises mycophenolate mofetil, azathioprine, and/or methotrexate. In some embodiments, the background immunotherapy comprises mycophenolate mofetil. In some embodiments, the background immunotherapy comprises azathioprine. In some embodiments, the background immunotherapy comprises methotrexate. In some embodiments, the background immunotherapy does not comprise hydroxychloroquine. In some embodiments, the background immunotherapy comprises oral corticosteroids, e.g., oral prednisone or equivalent. In some embodiments, the background immunotherapy comprises calcineurin inhibitors.

In one aspect, the invention features a method of treating an individual having systemic lupus erythematosus (SLE), comprising subcutaneously administering to the individual mosunetuzumab according to a dosing regimen comprising at least a first dosing cycle and a second dosing cycle, wherein: (a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg); and (b) the second dosing cycle comprises a single dose (C2D1) of mosunetuzumab, wherein the C2D1 of mosunetuzumab is about 45 mg (e.g., 45 mg±10%; 45 mg±4.5 mg), and wherein the method further comprises discontinuing administering a background immunotherapy to the individual.

In one aspect, the invention features a method of inducing B cell depletion in an individual, comprising subcutaneously administering to the individual mosunetuzumab according to a dosing regimen comprising nine 21-day dosing cycles, wherein: (a) the first dosing cycle comprises a first dose (C1D1), a second dose (C1D2), and a third dose (C1D3), wherein the C1D1 of mosunetuzumab is about 1.6 mg (e.g., 1.6 mg±10%; 1.6 mg±0.16 mg), the C1D2 of mosunetuzumab is about 5 mg (e.g., 5 mg±10%; 5 mg±0.5 mg), and the C1D3 of mosunetuzumab is about 45 mg (e.g., 45 mg±10%; 45 mg±4.5 mg), and wherein the C1D1, the C1D2, and the C1D3 are administered on Days 1, 8, and 15, respectively, of the first dosing cycle; and (b) the second to ninth dosing cycles each comprises a single dose (C2D1-C9D1) of mosunetuzumab administered on Day 1 of each respective dosing cycle, wherein each of the C2D1-C9D1 is about 45 mg (e.g., 45 mg±10%; 45 mg±4.5 mg), and wherein the method further comprises discontinuing administering a background immunotherapy to the individual.

In some embodiments, the individual has B cell depletion after dosing the individual with mosunetuzumab before reducing or removing background immunotherapy to the individual. In some embodiments, the B cell depletion is a deep-tissue B cell depletion and/or a peripheral B cell depletion. In some embodiments, the deep-tissue B cell depletion is determined by tissue biopsy. In some embodiments, the deep-tissue B cell depletion is determined by kidney biopsy. In some embodiments, the peripheral B cell depletion is determined by blood flow cytometry. In some embodiments, the amount of B cell depletion is determined relative to a reference level of B cells. In some embodiments, the reference level of B cells is determined at a time prior to administration of any dose of mosunetuzumab. In some embodiments, the background immunotherapy comprises at least one selected from the group consisting of mycophenolate mofetil, azathioprine, and methotrexate. In some embodiments, the background immunotherapy comprises mycophenolate mofetil, azathioprine, and/or methotrexate. In some embodiments, the background immunotherapy comprises mycophenolate mofetil. In some embodiments, the background immunotherapy comprises azathioprine. In some embodiments, the background immunotherapy comprises methotrexate.

In some embodiments, the background immunotherapy does not comprise hydroxychloroquine. In some embodiments, the background immunotherapy comprises oral corticosteroids, e.g., oral prednisone or equivalent. In some embodiments, the background immunotherapy comprises calcineurin inhibitors. In some embodiments, discontinuation of the background immunotherapy (e.g., oral corticosteroids and/or calcineurin inhibitors) begins at or about (e.g., within 1, 2, 3, 4, 5, 6, 7, 8, 9, 10, 11, 12, 13, 14, or more days) of the dosing regimen. In some embodiments, discontinuation of the background immunotherapy (e.g., oral corticosteroids and/or calcineurin inhibitors) begins any Day of any dosing cycle. In some embodiments, discontinuation of the background immunotherapy (e.g., oral corticosteroids and/or calcineurin inhibitors) begins any Day of the first dosing cycle. In some embodiments, discontinuation of the background immunotherapy (e.g., oral corticosteroids and/or calcineurin inhibitors) begins on any of Days 1, 2, 3, 4, 5, 6, 7, 8, 9, 10, 11, 12, 13, or 14 of the first dosing cycle. In some embodiments, discontinuation of the background immunotherapy (e.g., oral corticosteroids and/or calcineurin inhibitors) begins on any of Days 1, 2, 3, 4, 5, 6, or 7 of the first dosing cycle. In some embodiments, discontinuation of oral corticosteroids begins on Day 1 of the first dosing cycle. Additional factors which may affect the initiation of discontinuation of background immunotherapy includes the stability of the kidney biopsy chronicity index in patients with lupus nephritis, normalization of complement levels, and improvement in SLE-specific autoantibody levels.

In some embodiments, the background immunotherapy (e.g., of mycophenolate mofetil, azathioprine, and/or methotrexate) is to be discontinued suddenly, e.g., wherein administration of the background immunotherapy is stopped. In some embodiments, the background immunotherapy (e.g., oral corticosteroids and/or calcineurin inhibitors) is discontinued gradually, e.g., wherein administration of the background immunotherapy (e.g., oral corticosteroids and/or calcineurin inhibitors) is reduced (e.g., gradually reduced) from the original dose down to none or zero.

In some embodiments, discontinuation of oral corticosteroids (e.g., oral prednisone or equivalent) is decreased according to the schedule set forth in Table 9 below. In some embodiments, discontinuation of oral corticosteroids (e.g., oral prednisone or equivalent) originally administered at a dose of 10 mg per day proceeds according to: 10 mg per day in week 1, 7.5 mg per day in week 2, 7.5 mg per day in week 4, 5 mg per day in week 8, 5 mg per day in week 12, and 0 mg per day in week 16. In some embodiments, discontinuation of oral corticosteroids (e.g., oral prednisone or equivalent) originally administered at a dose of 20 mg per day proceeds according to: 20 mg per day in week 1, 15 mg per day in week 2, 10 mg per day in week 4, 7.5 mg per day in week 8, 5 mg per day in week 12, 5 mg per day in week 16, and 0 mg per day in week 20. In some embodiments, discontinuation of oral corticosteroids (e.g., oral prednisone or equivalent) originally administered at a dose of 30 mg per day proceeds according to: 30 mg per day in week 1, 25 mg per day in week 2, 20 mg per day in week 4, 15 mg per day in week 8, 10 mg per day in week 12, 7.5 mg per day in week 16, 5 mg per day in week 20, and 0 mg per day in week 24. In some embodiments, discontinuation of oral corticosteroids (e.g., oral prednisone or equivalent) originally administered at a dose of 40 mg per day proceeds according to: 40 mg per day in week 1, 30 mg per day in week 2, 20 mg per day in week 4, 15 mg per day in week 8, 10 mg per day in week 12, 7.5 mg per day in week 16, 5 mg per day in week 20, and 0 mg per day in week 24. In some embodiments, discontinuation of oral corticosteroids (e.g., oral prednisone or equivalent) originally administered at a dose of 50 mg per day proceeds according to: 50 mg per day in week 1, 40 mg per day in week 2, 30 mg per day in week 4, 20 mg per day in week 8, 10 mg per day in week 12, 7.5 mg per day in week 16, 5 mg per day in week 20, and 0 mg per day in week 24.

### B. Dosing Strategies for Mitigating Adverse Events

The present invention relates to methods of treating an individual having a systemic lupus erythematosus (SLE) by administration of mosunetuzumab. In some embodiments, therapies and dosing regimens described herein provide acceptable safety profiles in individuals with SLE treated with the described dosing regimens.

### 1. CRS Symptoms and Grading

Any of the methods described herein may involve monitoring an individual for cytokine release syndrome (CRS), e.g., a CRS event following commencement of any of the methods described above. Current clinical management focuses on treating the individual signs and symptoms, providing supportive care, and attempting to dampen the inflammatory response using a high dose of corticosteroids. However, this approach is not always successful, especially in the case of late intervention. The CRS grading criteria used by the methods described herein are published by the American Society for Transplantation and Cellular Therapy (ASTCT) to define mild, moderate, severe, or life-threatening CRS and harmonize reporting across clinical trials to allow rapid recognition and treatment of CRS (Lee et al. Biol Blood Marrow Transplantation. 25(4): 625-638, 2019). The ASTCT criteria is intended to be objective, easy to apply, and more accurately categorize the severity of CRS. This CRS grading system is shown below in Table 1.

TABLE 1
CRS Grading System

| CRS Parameter | Grade 1 | Grade 2 | Grade 3 | Grade 4 |
|---|---|---|---|---|
| Fever | Temperature ≥38° C. | Temperature ≥38° C. | Temperature ≥38° C. | Temperature ≥38° C. |
| | | with | | |
| Hypotension | None | Not requiring | Requiring a | Requiring multiple |
| | | vasopressors | vasopressor with or | vasopressors |
| | | | without vasopressin | (excluding |
| | | | | vasopressin) |
| | | and/or | | |
| Hypoxia | None | Requiring low-glow | Requiring high-flow | Requiring positive |
| | | nasal cannula or | nasal cannula, | pressure |
| | | blow-by | facemask, | (e.g., CPAP, BiPAP, |
| | | | nonrebreather mask | intubation and |
| | | | or Venturi mask | mechanical |
| | | | | ventilation) |

ASTCT = American Society for Transplantation and Cellular Therapy; BiPAP = bilevel positive airway pressure; CPAP = continuous positive airway pressure; CRS = cytokine release syndrome; CTCAE = Common Terminology Criteria for Adverse Events.

Fever is defined as a temperature ≥38° C. not attributable to any other cause. In individuals who have CRS then receive antipyretic or anticytokine therapy such as tocilizumab or corticosteroids, fever is no longer required to grade subsequent CRS severity. In this case, CRS grading is determined by hypotension and/or hypoxia.

CRS grade is determined by the more severe event, hypotension or hypoxia not attributable to any other cause. For example, an individual with temperature of 39.5° C., hypotension requiring 1 vasopressor, and hypoxia requiring low-flow nasal cannula is classified as Grade 3 CRS.

Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

CRS is associated with elevations in a wide array of cytokines, including marked elevations in IFN-γ, IL-6, and TNF-α levels. Emerging evidence implicates IL-6, in particular, as a central mediator in CRS. IL-6 is a proinflammatory, multi-functional cytokine produced by a variety of cell types, which has been shown to be involved in a diverse array of physiological processes, including T cell activation. Regardless of the inciting agent, CRS is associated with high IL-6 levels (Nagorsen et al. Cytokine. 25(1): 31-5, 2004; Lee et al. Blood. 124(2): 188-95, 2014); Doesegger et al. Clin. Transl. Immunology. 4(7): e39, 2015), and IL-6 correlates with the severity of CRS, with individuals who experience a Grade 4 or 5 CRS event having much higher IL-6 levels compared to individuals who do not experience CRS or experience milder CRS (Grades 0-3) (Chen et al. J. Immunol. Methods. 434:1-8, 2016).

Therefore, blocking the inflammatory action of IL-6 using an agent that inhibits IL-6-mediated signaling to manage CRS observed in individuals during the double-step fractionated, dose-escalation dosing regimen is an alternative to steroid treatment that would not be expected to negatively impact T cell function or diminish the efficacy or clinical benefit of mosunetuzumab therapy in the treatment of CD20-positive cell proliferative disorders (e.g., a B cell proliferative disorders).

If the individual has a CRS event that does not resolve or worsens within 24 hours of administering the IL-6R antagonist to treat the symptoms of the CRS event, and the method may further comprise administering to the individual one or more additional doses of the IL-6R antagonist to manage the CRS event. The individual may be administered a corticosteroid, such as methylprednisolone or dexamethasone if CRS event is not managed through administration of the IL-6R antagonist.

2. Dosing Regimens with Acceptable Safety Profiles

In some embodiments, subcutaneous administration of mosunetuzumab to an individual (e.g., an individual having an SLE) may result in the individual experiencing an adverse event, e.g., a cytokine release syndrome (CRS) event. Described herein are methods for administering one or more additional therapeutic agents for mitigating (e.g., reducing the severity or duration of), reducing the likelihood of, and/or preventing adverse events (e.g., CRS) in an individual.

In some embodiments, the individual is administered tocilizumab if the individual experiences CRS. In some embodiments, tocilizumab is administered to the subject as a single dose of about 8 mg/kg (e.g., 8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, 0.75 mg/kg, ±1 mg/kg, ±1.5 mg/kg, or ±2 mg/kg; e.g., 8 mg/kg), and wherein the single dose does not exceed 800 mg. In some embodiments, tocilizumab is administered to the subject as a single dose of about 12 mg/kg (e.g., 12 mg/kg±10%; 12 mg/kg±0.12 mg/kg), and wherein the single dose does not exceed 800 mg. In some embodiments, tocilizumab is administered intravenously. In some embodiments, a maximum of three doses of tocilizumab is administered in any six-week period. In some embodiments, if an individual is refractory to tocilizumab, the individual is administered siltuximab (CAS #: 541502-14-1), anakinra (CAS #: 143090-92-0), dasatinib (302962-49-8), or emapalumab (CAS #: 1709815-23-5).

In some embodiments, the individual is administered a corticosteroid if the individual experiences CRS. In some embodiments, the corticosteroid administered to treat a CRS event is dexamethasone or methylprednisolone. In some embodiments, dexamethasone is administered intravenously at a dose of about 10 mg (e.g., 10 mg±10%; 10 mg±1 mg), e.g., to treat a CRS event. In some embodiments, dexamethasone is administered intravenously at a dose of about 10 mg (e.g., 10 mg±10%; 10 mg±1 mg) every six hours until the CRS event resolves. In some embodiments, the individual is refractory to dexamethasone, the individual is administered methylprednisolone, e.g., to treat the CRS event. In some embodiments, methylprednisolone is intravenously administered at a dose of about 1000 mg/day (e.g., 1000 mg/day±10%; 1000 mg/d±100 mg/day), e.g., to treat a CRS event.

Additional treatment details for CRS events are described below in Table 2.

TABLE 2
Management of CRS for Individuals Receiving Subcutaneous Mosunetuzumab

| | | Anti-IL-6/Corticosteroid | |
|---|---|---|---|
| CRS Grade^{a} | Supportive Care | Therapy | Action for Next Dose |
| Grade 1 fever ≥38° C.^{b} | Provide symptomatic | For prolonged CRS (>2 | May receive the next dose |
| | management of | days) in participants with | of mosunetuzumab if |
| | constitutional | significant symptoms | symptoms resolve for 3 |
| | symptoms and organ | and/or comorbidities (per | consecutive days |
| | toxicities | investigator's discretion | Administer premedications |
| | Consider empiric | [e.g., impaired | for next dose |
| | broad-spectrum | cardiovascular function, | The individual should be |
| | antibiotics | reduced pulmonary | monitored more frequently |
| | Consider G-CSF if | reserve]), consider | |
| | patient is neutropenic | tocilizumab^{c} and | |
| | Maintenance IV fluids | corticosteroids as per | |
| | for hydration | Grade 2 | |
| | Consider | | |
| | hospitalization until | | |
| | symptoms completely | | |
| | resolve | | |
| Grade 2 fever ≥38° C. | Provide symptomatic | Consider tocilizumab^{c} | May receive the next dose |
| b with hypotension | management of | For persistent refractory | of mosunetuzumab if |
| not requiring | constitutional | hypotension after 1 or 2 | symptoms resolve to Grade |
| vasopressors and/or | symptoms and organ | doses of anti-IL-6 | 1 or better for 3 consecutive |
| hypoxia requiring | toxicities | therapy, consider 10 mg | days |
| low-flow oxygend by | Consider ICU | IV dexamethasone every | Premedications should be |
| nasal cannula or | admission for | 6 hours (or equivalent) | maximized as appropriate |
| blow-by | hemodynamic | Manage per Grade 3 if | Consider hospitalization of |
| | monitoring | no improvement within | the individual for next dose |
| | For hypotension: IV | 24 hours after starting | |
| | fluid bolus as | tocilizumab | |
| | needed; for persistent | | |
| | refractory | | |
| | hypotension (e.g., | | |
| | after 2 fluid boluses | | |
| | and anti-IL-6 | | |
| | therapy), start | | |
| | vasopressors and | | |
| | manage per Grade 3 | | |
| | Rule out other | | |
| | inflammatory | | |
| | conditions, which can | | |
| | mimic severe CRS | | |
| | (e.g., infections/ | | |
| | sepsis), undertake | | |
| | SARS-CoV-2 | | |
| | diagnostic PCR | | |
| | Consider empiric | | |
| | broad-spectrum | | |
| | antibiotics | | |
| | If no improvement | | |
| | within 24 hours, | | |
| | initiate work-up and | | |
| | assess for signs and | | |
| | symptoms of HLH | | |
| Grade 3 fever ≥38° C. | Symptomatic | Administer tocilizumab^{c} | May receive the next dose |
| b with hypotension | management of | Dexamethasone 10 mg | of mosunetuzumab if CRS |
| requiring a | organ toxicities, admit | IV every 6 hours (or | event was responsive to |
| vasopressor (with or | patient to ICU for | equivalent) | treatment (i.e., clinical |
| without vasopressin) | hemodynamic | If CRS is refractory to | improvement within 8-12 |
| and/or hypoxia | monitoring | tocilizumab and | hours following |
| requiring high-flow | For hypotension: IV | dexamethasone, | tocilizumab/corticosteroids |
| oxygen^{d} by nasal | fluid bolus and | manage as per Grade 4 | administration) and |
| cannula, face mask, | vasopressors as | with alternative | symptoms resolve to Grade |
| nonrebreather mask, | needed | immunosuppressants | 1 or better for 3 consecutive |
| or Venturi-mask | Rule out other | and 1000 mg/day | days |
| | inflammatory | methylprednisolone | Premedications should be |
| | conditions that can | | maximized as appropriate |
| | mimic severe CRS | | Hospitalize patient for next |
| | (e.g., infections or | | dose |
| | sepsis), undertake | | If the event occurs after |
| | SARS-CoV-2 | | 1.6 mg, the next dose |
| | diagnostic PCR | | should be 1.6 mg, i.e. the |
| | Consider empiric | | treatment should be re- |
| | broad-spectrum | | started |
| | antibiotics | | If the event occurs after 5 |
| | If no improvement | | mg or 45 mg, the next dose |
| | within 24 hours, | | should be 5 mg |
| | initiate work-up and | | If the next dose is tolerated |
| | assess for signs and | | without Grade ≥3 CRS, the |
| | symptoms of HLH | | individual may return to the |
| | | | originally planned dose |
| | | | If Grade 3 CRS recurs with |
| | | | subsequent doses, |
| | | | consider permanent |
| | | | discontinuation of |
| | | | mosunetuzumab |
| Grade 4 fever ≥38° C. | ICU admission and | Administer tocilizumab c | Permanently discontinue |
| b with hypotension | hemodynamic | For individuals refractory | mosunetuzumab ^{h} |
| requiring multiple | monitoring | to tocilizumab, consider | |
| vasopressors | Mechanical | siltuximab, anakinra, | |
| (excluding | ventilation as needed | dasatinib, and | |
| vasopressin) and/or | IV fluids and | emapalumab, based on | |
| hypoxia requiring | vasopressors as | discretion of the | |
| oxygen by positive | needed | investigator^{e} | |
| pressure (e.g., C- | Symptomatic | Administer 10 mg IV | |
| PAP, BiPAP-, | management of | dexamethasone every 6 | |
| intubation, and | organ toxicities | hours (or equivalent) | |
| mechanical | Rule out other | If refractory, consider | |
| ventilation) | inflammatory | 1000 mg/day IV | |
| | conditions that can | methylprednisolone ^{f, g} | |
| | mimic severe CRS | | |
| | (e.g., infections or | | |
| | sepsis), undertake | | |
| | SARS-CoV-2 | | |
| | diagnostic PCR | | |
| | Consider empiric | | |
| | broad-spectrum | | |
| | antibiotics | | |
| | If no improvement | | |
| | within 24 hours, | | |
| | initiate work up and | | |
| | assess for signs and | | |
| | symptoms HLH | | |

AE = adverse event; ASTCT = American Society for Transplantation and Cellular Therapy; BiPAP = bilevel positive airway pressure; C-PAP = continuous positive airway pressure; CRS = cytokine release syndrome; G-CSF = granulocyte colony stimulating factor; HLH = hemophagocytic lymphohistiocytosis, ICU = intensive care unit; IL-6 = interleukin 6; IRR = infusion-related reaction; IV = intravenous; PCR = polymerase chain reaction; SARS-CoV-2 = severe acute respiratory syndrome coronavirus 2.
^{a}Cytokine release syndrome will be assessed according to the ASTCT Consensus Grading Criteria (Lee et al. Biol Blood Marrow Transplantation. 25(4): 625-638, 2019).
Fever is defined as temperature ≥38° C. not attributable to any other cause. In individuals who have CRS and then receive antipyretic or anti-cytokine therapy such as tocilizumab or steroids, fever is no longer required to grade subsequent CRS severity. CRS grade is determined by the more severe event: hypotension or hypoxia not attributable to any other cause.
^{b}As individuals may have received corticosteroid premedication, a fever response may be blunted. Therefore, adverse events attributed to mosunetuzumab consistent with a diagnosis of IRR or CRS, and associated with fever, hypotension or hypoxia not attributable to any other cause, are recorded as CRS. Cytokine release syndrome events that manifest with hypotension and/or hypoxia, but with no fever, are graded depending on management required for hypotension and/or hypoxia. These types of CRS events correspond to minimum ASTCT Grade 2. Other adverse events occurring within 48 hours after mosunetuzumab administration are reported as individual adverse events (e.g., headache or chills).
^{c}Tocilizumab should be administered at a dose of 8 mg/kg IV (not exceeding 800 mg per infusion). If no clinical improvement in the signs and symptoms of CRS occurs after the first dose, a second dose may be administered at least 8 hours apart (maximum 2 doses per CRS event). Within each time-period of 6 weeks of mosunetuzumab treatment, the total number of tocilizumab doses should not exceed 3 doses.
^{d}Low-flow nasal cannula is defined as oxygen delivered at ≤6 L/min. Low flow also includes blow-by oxygen delivery. High-flow nasal cannula is defined as oxygen delivered at >6 L/min.
^{e}Riegler et al. Ther. Clin. Risk Manag. 15: 323-335, 2019.
^{f} Antifungal prophylaxis is strongly considered in individuals receiving steroids for treatment of CRS.
^{g} For example, 1000 mg/day IV methylprednisolone for 3 days, followed by rapid taper at 250 mg every 12 hours for 2 days, 125 mg every 13 hours for 2 days, and 60 mg every 12 hours for 2 days.
^{h} Resumption of mosunetuzumab may be considered in individuals who are deriving benefit and have fully recovered from the adverse event. Further treatment will not be considered unless all the criteria below are met: Individual risk-benefit assessment by Principal Investigator/treating physician favors continued treatment; the individual has recovered from previous toxicities and has sufficient organ function/reserve to receive subsequent doses; the individual has been adequately consented for risks associated with continued treatment and decides to receive subsequent doses; subsequent doses are well planned with precautionary measures, including dose reduction,, mandatory hospitalizations, and enhanced premedications.

In some embodiments, the individual is administered dexamethasone, diphenhydramine, and/or acetaminophen/paracetamol prior to administration of any dose of mosunetuzumab. In some embodiments, dexamethasone, diphenhydramine, and/or acetaminophen/paracetamol are pre-administered prior to any dose of mosunetuzumab. In some embodiments, dexamethasone, diphenhydramine, and/or acetaminophen/paracetamol are pre-administered prior to each dose of mosunetuzumab. In some embodiments, the individual is administered dexamethasone, diphenhydramine, and acetaminophen/paracetamol prior to administration of the C1D1, C1D2, C1D3, and C2D1 of mosunetuzumab. In some embodiments, dexamethasone is administered orally at a dose of about 20 mg (e.g., 20 mg±10%; 20 mg±2 mg). In some embodiments, diphenhydramine is administered orally at a dose of about 25 mg to about 50 mg (e.g., 25 mg to 45 mg, 25 mg to 40 mg, 25 mg to 35 mg, 25 mg to 30 mg, 30 mg to 50 mg, 40 mg to 50 mg, 45 mg to 50 mg, 30 mg to 35 mg, 30 mg to 45 mg, 35 mg to 45 mg, 40 mg to 45 mg, or 30 mg to 40 mg; e.g., about 25 mg, about 30 mg, about 35 mg, about 40 mg, about 45 mg, or about 50 mg). In some embodiments, acetaminophen/paracetamol is administered orally at a dose of about 650 mg to about 1000 mg (e.g., 650 mg to 900 mg, 650 mg to 80 mg, 650 mg to 700 mg, 700 mg to 1000 mg, 800 mg to 1000 mg, 900 mg to 1000 mg, 700 mg to 800 mg, 800 mg to 900 mg, 700 mg to 900 mg; e.g., about 650 mg, about 700 mg, about 750 mg, about 800 mg, about 850 mg, about 900 mg, about 950 mg, or about 1000 mg). In some embodiments, dexamethasone is administered orally at a dose of about 20 mg (e.g., 20 mg±10%; 20 mg±2 mg); diphenhydramine is administered orally at a dose of about 25 mg to about 50 mg (e.g., 25 mg to 45 mg, 25 mg to 40 mg, 25 mg to 35 mg, 25 mg to 30 mg, 30 mg to 50 mg, 40 mg to 50 mg, 45 mg to 50 mg, 30 mg to 35 mg, 30 mg to 45 mg, 35 mg to 45 mg, 40 mg to 45 mg, or 30 mg to 40 mg; e.g., about 25 mg, about 30 mg, about 35 mg, about 40 mg, about 45 mg, or about 50 mg); and acetaminophen/paracetamol is administered orally at a dose of about 650 mg to about 1000 mg (e.g., 650 mg to 900 mg, 650 mg to 80 mg, 650 mg to 700 mg, 700 mg to 1000 mg, 800 mg to 1000 mg, 900 mg to 1000 mg, 700 mg to 800 mg, 800 mg to 900 mg, 700 mg to 900 mg; e.g., about 650 mg, about 700 mg, about 750 mg, about 800 mg, about 850 mg, about 900 mg, about 950 mg, or about 1000 mg).

### IV. Therapeutic Agents

### A. Mosunetuzumab

The invention provides mosunetuzumab, a bispecific antibody that binds to CD20 and CD3, useful for treating a systemic lupus erythematosus (SLE).

Mosunetuzumab has an anti-CD20 arm having a first binding domain comprising the following six hypervariable regions (HVRs): (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6). Mosunetuzumab comprises an anti-CD20 arm comprising a first binding domain comprising the heavy chain framework regions FR-H1, FR-H2, FR-H3, and FR-H4 comprising the sequences of SEQ ID NOs: 17-20, respectively, and the light chain framework regions FR-L1, FR-L2, FR-L3, and FR-L4 comprising the sequences of SEQ ID NOs: 21-24, respectively. Mosunetuzumab comprises an anti-CD20 arm comprising a first binding domain comprising (a) a heavy chain variable (VH) domain comprising the amino acid sequence of SEQ ID NO: 7; and (b) a light chain variable (VL) domain comprising the amino acid sequence o, SEQ ID NO: 8.

Mosunetuzumab has an anti-CD3 arm having a second binding domain comprising the following six HVRs: (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). Mosunetuzumab comprises an anti-CD3 arm comprising a second binding domain comprising the heavy chain framework regions FR-H1, FR-H2, FR-H3, and FR-H4 comprising the sequences of SEQ ID NOs: 25-28, respectively, and the light chain framework regions FR-L1, FR-L2, FR-L3, and FR-L4 comprising the sequences of SEQ ID NOs: 29-32, respectively. Mosunetuzumab comprises an anti-CD3 arm comprising a second binding domain comprising (a) a VH domain comprising an amino acid sequence having the amino acid sequence of SEQ ID NO: 15; and (b) a VL domain comprising the amino acid sequence of SEQ ID NO: 16.

Mosunetuzumab has the International Nonproprietary Names for Pharmaceutical Substances (INN) List 117 (WHO Drug Information, Vol. 31, No. 2, 2017, p. 303-305), or CAS Registry No. 1905409-39-3, and has (1) an anti-CD20 arm comprising the heavy chain and light chain sequences of SEQ ID NOs: 33 and 34, respectively; and (2) an anti-CD3 arm comprising the heavy chain and light chain sequences of SEQ ID NOs: 35 and 36, respectively. Mosunetuzumab comprises (1) an anti-CD20 arm comprising a first binding domain comprising a heavy chain comprising the amino acid sequence of SEQ ID NO: 33 and a light chain comprising the amino acid sequence of SEQ ID NO: 34 and (2) an anti-CD3 arm comprising a second binding domain comprising a heavy chain comprising the amino acid sequence of SEQ ID NO: 35 and a light chain comprising the amino acid sequence of SEQ ID NO: 36.

Amino acid sequences of mosunetuzumab are summarized in Table 3 below.

TABLE 3
Sequence IDs for Mosunetuzumab

| CD3 Arm | | CD20 Arm | |
|---|---|---|---|
| SEQ ID NO: | Description | SEQ ID NO: | Description |
| 9 | CD3 HVR-H1 | 1 | CD20 HVR-H1 |
| 10 | CD3 HVR-H2 | 2 | CD20 HVR-H2 |
| 11 | CD3 HVR-H3 | 3 | CD20 HVR-H3 |
| 12 | CD3 HVR-L1 | 4 | CD20 HVR-L1 |
| 13 | CD3 HVR-L2 | 5 | CD20 HVR-L2 |
| 14 | CD3 HVR-L3 | 6 | CD20 HVR-L3 |
| 15 | CD3 VH | 7 | CD20 VH |
| 16 | CD3 VL | 8 | CD20 VL |
| 35 | CD3 heavy chain | 33 | CD20 heavy chain |
| 36 | CD3 light chain | 34 | CD20 light chain |

Mosunetuzumab may be produced using recombinant methods and compositions, for example, as described in U.S. Pat. No. 4,816,567.

### B. Additional Therapeutic Agents

In some instances, the methods described herein include administering mosunetuzumab in combination with one or more additional therapeutic agents.

In some instances, the one or more additional therapeutic agents may prevent, reduce the rate of, or reduce the severity of cytokine release syndrome (CRS) and/or symptoms associated with CRS. In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #: 50-42-8), or methylprednisolone (CAS #: 83-43-2)), antihistamine (e.g., diphenhydramine (CAS #: 58-73-1) and pharmaceutically acceptable salts thereof, e.g., diphenhydramine hydrochloride (CAS #: 147-24-0)) or an interleukin-6 receptor (IL-6R) antagonist (e.g., tocilizumab (CAS #: 375823-41-9), sarilumab (CAS #: 1189541-98-7), vobarilizumab (ALX-0061; CAS #: 1628814-88-9), satralizumab (SA-237; CAS #: 1535963-91-7), and alternatives thereof). In some instances, the additional therapeutic agent is a corticosteroid. In particular instances, the corticosteroid is dexamethasone (CAS #: 50-02-2), prednisone (CAS #: 53-03-2), prednisolone (CAS #: 50-42-8), hydrocortisone (CAS #: 50-23-7), cortisone acetate (CAS #: 50-04-4), betamethasone (CAS #: 378-44-9), triamcinolone (CAS #: 124-94-7), beclomethasone (CAS #: 4419-39-0), or methylprednisolone (CAS #: 83-43-2).

In some instances, the additional therapeutic agent is an antihistamine. In a particular instance, the antihistamine is diphenhydramine hydrochloride (CAS #: 58-73-1).

In some instances, the additional therapeutic agent is an antipyretic. In a particular instance, the antipyretic is acetaminophen (i.e., paracetamol; CAS #: 103-90-2) or a pharmaceutically acceptable salt thereof.

In some instances, the additional therapeutic agent is an IL-6R antagonist. In a particular instance, the IL-6R antagonist is tocilizumab (CAS #: 375823-41-9).

In some instances, the one or more additional therapeutic agents may be an antimalarial agent.

In particular instances, the additional therapeutic agent is hydroxychloroquine (CAS #: 118-42-3), chloroquine (CAS #: 54-05-7), or quinacrine (CAS #: 83-89-6).

In some instances, the one or more additional therapeutic agents may be an immunosuppressant. In particular instances, the additional therapeutic agent is azathioprine (CAS #: 446-86-6), mycophenolate mofetil (CAS #: 128794-94-5), mycophenolic acid (CAS #: 24280-93-1), or methotrexate (CAS #: 59-05-2).

In some instances, the one or more additional therapeutic agents may be a B-cell-depleting therapy. In particular instances, the additional therapeutic agent is rituximab (CAS #: 174722-31-7), ocrelizumab (CAS #: 637334-45-3), ofatumumab (CAS #: 679818-59-8), or obinutuzumab (CAS #: 949142-50-1).

In some instances, the one or more additional therapeutic agents may be a calcineurin inhibitor. In particular instances, the additional therapeutic agent is ciclosporin (CAS #: 59865-13-3), tacrolimus (CAS #: 104987-11-3), or vocolosporin (CAS #: 515814-01-4).

In some instances, the one or more additional therapeutic agents may be an alkylating agent. In particular instances, the additional therapeutic agent is cyclophosphamide (CAS #: 50-18-0).

In some instances, the one or more additional therapeutic agents may be an anti-CD19 therapy. In particular instances, the additional therapeutic agent is blinatumomab (CAS #: 853426-35-4).

In some instances, the one or more additional therapeutic agents may be an anti-CD20 therapy. In particular instances, the additional therapeutic agent is obinutuzumab (CAS #: 949142-50-1), rituximab (CAS #: 174722-31-7), ocrelizumab (CAS #: 637334-45-3), or ofatumumab (CAS #: 679818-59-8).

In some instances, the one or more additional therapeutic agents may be a kinase inhibitor. In particular instances, the additional therapeutic agent is baricitinib (CAS #: 1187594-09-7), tofacitinib (CAS #: 477600-75-2), upadacitinib (CAS #: 1310726-60-3), filgotinib (CAS #: 1206161-97-8), ibrutinib (CAS #: 936563-96-1), or fenebrutinib (CAS #: 1434048-34-6).

In some instances, the one or more additional therapeutic agents may be a biologic therapy. In particular instances, the additional therapeutic agent is belimumab (CAS #: 356547-88-1), ustekinumab (CAS #: 815610-63-0), anifrolumab (CAS #: 1326232-46-5), secukinumab (CAS #: 875356-43-7), or atacicept (CAS #: 845264-92-8).

In some instances, the one or more additional therapeutic agents may be an advanced therapy. In particular instances, the additional therapeutic agent is cyclophosphamide (CAS #: 50-18-0), belimumab (CAS #: 356547-88-1), ustekinumab (CAS #: 815610-63-0), anifrolumab (CAS #: 1326232-46-5), secukinumab (CAS #: 875356-43-7), atacicept (CAS #: 845264-92-8), obinutuzumab (CAS #: 949142-50-1), rituximab (CAS #: 174722-31-7), ocrelizumab (CAS #: 637334-45-3), or ofatumumab (CAS #: 679818-59-8).

In some instances, the one or more additional therapeutic agents may be used in the treatment of neutropenia. In some instances, the additional therapeutic agents may prevent symptoms associated with neutropenia. In some instances, the additional therapeutic agents may reduce the rate or severity of neutropenia. In particular instances, the additional therapeutic agent is granulocyte colony-stimulating factor (G-CSF or GCSF) or colony-stimulating factor 3 (CSF 3). The mRNA sequence of human G-CSF/CSF 3 includes, e.g., NCBI RefSeq No. NM_000759, NM_001178147, NM_172219, and NM_172220, and the protein amino acid sequence of human G-CSF/CSF 3 includes, e.g., NCBI RefSeq No. NP_000750, NP_001171618, NP_757373, and NP_757374.

### IV. Pharmaceutical Compositions and Formulations

Any of the antibodies (e.g., anti-CD20/anti-CD3 bispecific antibodies, e.g., mosunetuzumab) and/or other therapeutic agents described herein can be used in pharmaceutical compositions and formulations. Pharmaceutical compositions and formulations of antibodies and/or other therapeutic agents describe herein can be prepared by mixing one, two, or all three agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and rm-cresol): low molecular weight (less than about 10 residues) polypeptides: proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. In some instances, mosunetuzumab may be provided in a pharmaceutical composition as disclosed in U.S. Pat. No. 11,958,906. In some instances, mosunetuzumab is provided as a pharmaceutical composition comprising 1 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some instances, the 1 mg/ml pharmaceutical composition comprises 1 mg of mosunetuzumab diluted in 1 ml liquid. In some instances, mosunetuzumab is provided as a pharmaceutical composition comprising 10 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some instances, the 10 mg/ml pharmaceutical composition comprises 5 mg of mosunetuzumab diluted in 0.5 ml liquid. In some instances, mosunetuzumab is provided as a pharmaceutical composition comprising 45 mg/ml mosunetuzumab, about 10 mM L-histidine, about 240 mM sucrose, about 0.06% (w/v) PS20, and about 10 mM methionine, wherein the pharmaceutical composition has a pH of about 5.8. In some instances, the 45 mg/ml pharmaceutical composition comprises 45 mg of mosunetuzumab diluted in 1 ml liquid. In some instances, the pharmaceutical composition further comprises histidine acetate. In some instances, the 1 mg/ml mosunetuzumab pharmaceutical composition is used to administer mosunetuzumab at a dose of 1.6 mg (e.g., administering 1.6 ml of the 1 mg/ml pharmaceutical composition). In some instances, the 10 mg/ml mosunetuzumab pharmaceutical composition is used to administer mosunetuzumab at a dose of 1.6 mg (e.g., administering 0.16 ml of the 10 mg/al pharmaceutical composition). In some instances, the 10 mg/ml mosunetuzumab pharmaceutical composition is used to administer mosunetuzumab at a dose of 5 mg (e.g., administering 0.5 ml of the 10 mg/mil pharmaceutical composition). In some instances, the 45 mg/ml mosunetuzumab pharmaceutical composition is used to administer mosunetuzumab at a dose of 45 mg (e.g., administering 1 ml of the 45 mg/mil pharmaceutical composition).

The formulation herein may also contain more than one active ingredient as necessary for the particular indication being treated, preferably those with complementary activities that do not adversely affect each other. For example, it may be desirable to further provide an additional therapeutic agent (e.g., IL-6R antagonist, corticosteroid, antimalarial, antipyretic, cyclophosphamides, B-cell-depleting therapies, and calcineurin inhibitors, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody and/or therapeutic agent, which matrices are in the form of shaped articles, for example, films, or microcapsules.

The formulations to be used for in vivo administration are generally sterile. Sterility may be readily accomplished, e.g., by filtration through sterile filtration membranes. In some embodiments, mosunetuzumab is formulated for administration subcutaneously. In some embodiments, dexamethasone is formulated for administration orally or intravenously. In some embodiments, methylprednisolone is formulated for administration orally or intravenously. In some embodiments, prednisone is formulated for administration orally. In some embodiments, tocilizumab is formulated for administration intravenously.

### V. Kits and Articles of Manufacture

In another aspect of the invention, a kit or an article of manufacture containing materials useful for the treatment, prevention, and/or diagnosis of the disorders described above (e.g., systemic lupus erythematosus (SLE)) is provided. The kit or article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is mosunetuzumab described herein. The label or package insert indicates that the composition is used for treating systemic lupus erythematosus (SLE) and further includes information related to at least one of the dosing regimens described herein. In some embodiments, the label or package insert indicates that the composition is used for treating SLE in an individual. Moreover, the kit or article of manufacture may comprise (a) a first container with a composition contained therein, wherein the composition comprises mosunetuzumab; and (b) a second container with a composition contained therein, wherein the composition comprises an additional therapeutic agent described herein. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### Example 1. A Phase II Open-Label Study to Evaluate the Efficacy, Safety, and Pharmacokinetics of Mosunetuzumab in Patients with Systemic Lupus Erythematosus with or without Active Lupus Nephritis

### A. Introduction

### Purpose of Trial

The GA45799 study provided in these Examples evaluates the efficacy, safety, and pharmacokinetics (PK) of mosunetuzumab, a novel anti-CD20 bispecific T cell engager that binds to cluster of differentiation 20 (CD20) and cluster of differentiation 3 (CD3), in patients with active lupus nephritis (LN) (Cohort 1) and active systemic lupus (Cohort 2). The primary efficacy objective is to evaluate whether mosunetuzumab can confer enough deep tissue and peripheral B-cell depletion to induce sustained drug-free disease remission.

As a T cell-recruiting bispecific antibody, mosunetuzumab is a conditional agonist; target B cell killing is observed only upon simultaneous binding to CD20 on B cells and CD3 on T cells. Engagement of both arms of mosunetuzumab results in the formation of an immunologic synapse between a target B cell and a cytotoxic T cell resulting in T cell activation in a target- and dose-dependent manner. T cell activation is manifested by the expression of activation-related surface markers (e.g., CD69 and CD25), transient release of cytokines (e.g., interferon-[IFN-], tumor necrosis factor-[TNF-], interleukins [IL]-2, 6, and 10), and robust T cell proliferation. Subsequent directed release of perforin and a cocktail of granzymes from T cells through the immunologic synapse results in B cell lysis.

B cells play an active role in the pathogenesis of SLE. Autoreactive B cells recognize and present self-antigens, produce autoantibodies, secrete proinflammatory cytokines, and participate in T cell co-stimulation. For this reason, they are attractive therapeutic targets in SLE, although there have been a number of failed studies evaluating agents that target B cells, B cell activation, or B cell survival factors, including randomized controlled trials of the Type I anti-CD20 monoclonal antibody rituximab for treatment of SLE (Study U2971g [EXPLORER]) and severe LN (Study U2970g [LUNAR]).

Mosunetuzumab may achieve a greater degree of B cell depletion compared with other anti-CD20 molecules, based on the T cell-dependent mechanism, more extensive killing of tissue-infiltrating B cells, and more efficient killing of cells with very low levels of CD20 expression, which in turn may translate to improved efficacy. Mosunetuzumab is currently approved in many countries worldwide for the treatment of adult patients with relapsed/refractory (R/R) follicular lymphoma (FL) after two or more prior lines of systemic therapy. However, bispecific antibodies have never been previously tested for treating SLE patients having potentially disregulated immune systems, and so the performance of mosunetuzumab in treating SLE is unknown. A Phase 1b study (described below in Example 2) describes the first results obtained regarding the safety of an anti-CD20/anti-CD3 bispecific antibody, mosunetuzumab, when administered to patients with SLE, and also explores the pharmacodynamics of different dosing for mosunetuzumab empirically and in silico. However, for treating SLE patients, a safe and efficacious dosing regimen is needed.

### B. Primary, Secondary and Exploratory Objectives and Corresponding Endpoints

The GA45799 study provided in this Example is designed to assess the efficacy, safety, and pharmacokinetics (PK) of mosunetuzumab, a novel anti-CD20 bispecific T cell engager, in patients with active systemic lupus erythematosus (SLE) with or without active lupus nephritis (LN). Current standard-of-care therapies for SLE include oral corticosteroids (OCS), antimalarial therapies (such as hydroxychloroquine, chloroquine, and quinacrine), conventional immunosuppressants (such as mycophenolate mofetil (MMF), azathioprine, and methotrexate), and monoclonal antibodies (such as belimumab and anifrolumab). However, many patients adherent to this multiple-therapy regimen still often experience flares of active disease and thus remain vulnerable to untreated inflammation. As such, there continues to be a need for treatments with better benefit-risk profiles that attenuate inflammation and clinical sequelae and improve the long-term prognosis of people living with lupus.

### Primary Objective and Corresponding Endpoint

The primary objective for the GA45799 study is expressed using the estimand framework in accordance with the International Council for Harmonisation E9(R1) statistical principles for clinical trials (International Council for Harmonisation, E9 (R1) addendum on estimands and sensitivity analysis in clinical trials to the guideline on statistical principles for clinical trials, 2020) and are shown in Table 4, below.

TABLE 4
Primary Objective and Corresponding Endpoint

| Primary Objective | Corresponding Endpoint |
|---|---|
| To evaluate the efficacy | Achievement of drug-free remission by |
| of mosunetuzumab in | Week 76 |
| conferring remission | Drug-free Remission is defined as achieving |
| | both of the following: |
| | Absence of disease activity (Clinical |
| | Systemic Lupus Erythematosus Disease |
| | Activity Index (cSLEDAI; Bombardier, C. et |
| | al., Arthritis & Rheumatism, 35: 630-640, |
| | 1992) = 0) maintained for 6 months after |
| | completion of mosunetuzumab treatment |
| | Not receiving any systemic lupus |
| | erythematosus (SLE)-directed therapy |
| | (except for antimalarials) during the 6 |
| | months |
| | Doses of prednisone (or equivalent) ≤5 |
| | mg/day to treat secondary adrenal |
| | insufficiency are permitted |

### Secondary Objectives and Corresponding Endpoints

Secondary objectives and corresponding endpoints are described in Table 5 below.

TABLE 5
Secondary Objectives and Corresponding Endpoints

| Secondary Objectives | Corresponding Endpoints |
|---|---|
| To evaluate the efficacy of mosunetuzumab | Proportion of participants who achieve |
| in conferring clinical response (also known as | DORIS remission by Week 76, defined as |
| Definition of Remission in SLE | achievement of all of the following for at least |
| (DORIS; van Vollenhoven et al., Lupus Sci | 6 months: |
| Med., 9(1): e000538corr1, 2022) | cSLEDAI = 0 after completion of |
| | mosunetuzumab treatment |
| | Stable dosing of SLE therapies for at |
| | least 6 months |
| | Physician Global Assessment (PGA) |
| | of <0.5. PGA will be performed at Week 12, |
| | Week 24, and any time the cSLEDAI is |
| | assessed to be zero. If possible, a PGA is |
| | done during the screening period or at |
| | baseline before the first mosunetuzumab |
| | treatment. |
| | Prednisone (or equivalent) dose ≤5 |
| | mg/day |
| To evaluate the efficacy of mosunetuzumab | Proportion of participants who achieve CRR |
| in conferring complete renal response (CRR) | at Weeks 24, 52, 76, and 104 defined as all |
| (Cohort 1 only) | of the following: |
| | Urinary protein-to-creatinine ratio |
| | (UPCR) <0.5 g/g |
| | Estimated glomerular filtration rate |
| | (eGFR) ≥85% of baseline, as calculated |
| | using the Chronic Kidney Disease |
| | Epidemiology Collaboration (CKD-EPI; |
| | nih.gov) equation or ≥60 ml/min per 1.73 |
| | m² of body-surface area |
| To evaluate the efficacy of mosunetuzumab | Proportion of participants who achieve |
| in conferring partial renal response (PRR) | PRR at Weeks 24, 52, 76, and 104, |
| (Cohort 1 only) | defined as all of the following: |
| | ≥50% reduction in UPCR from baseline |
| | UPCR <1 g/g (or <3 g/g if the baseline |
| | UPCR was ≥3 g/g) |
| | eGFR ≥85% of baseline, as calculated |
| | using the CKD-EPI equation |
| To evaluate the safety of mosunetuzumab | Incidence and severity of adverse events, |
| | with severity determined according to |
| | National Cancer Institute Common |
| | Terminology Criteria for Adverse Events, |
| | Version 5.0 (NCI CTCAE v5.0; |
| | cancer.gov) and cytokine release |
| | syndrome (CRS) and immune effector |
| | cell-associated neurotoxicity syndrome |
| | (ICANS) severity determined according to |
| | the 2019 American Society for |
| | Transplantation and Cellular Therapy |
| | (ASTCT) CRS and ICANS Consensus |
| | grading criteria. |
| | Change from baseline in targeted vital |
| | signs |
| | Change from baseline in targeted clinical |
| | laboratory test results |
| To evaluate the effect of mosunetuzumab on | Longitudinal changes in titers of anti- |
| lupus disease activity biomarkers | dsDNA and anti-Smith antibodies |
| | Longitudinal changes in complement C3 |
| | and C4 |
| To characterize pharmacokinetic (PK) of | Serum concentrations of mosunetuzumab |
| mosunetuzumab | at specified timepoints |
| | Relevant PK parameters of |
| | mosunetuzumab |
| To evaluate the immunogenicity of | Prevalence of anti-drug antibodies |
| mosunetuzumab | (ADAs) at baseline and incidence of |
| | ADAs during the study |
| To evaluate the pharmacodynamic effects of | B cell levels in the blood (CD19+, |
| mosunetuzumab on circulating B cells | absolute counts in blood) at specified |
| | timepoints |
| To evaluate the efficacy of mosunetuzumab | Change in Functional Assessment of |
| on patient-reported outcome measures | Chronic Illness Therapy (FACIT) - |
| | Fatigue from baseline to Week 76 |
| | Change in subject global assessment |
| | (SGA) from baseline to Week 76 |

### Exploratory Objectives and Corresponding Endpoints

Exploratory objectives and corresponding endpoints are described in Table 6 below.

TABLE 6
Exploratory Objectives and Corresponding Endpoints

| Exploratory Objectives | Corresponding Endpoints |
|---|---|
| To identify and/or evaluate biomarkers | Longitudinal changes in phenotypic and |
| associated with susceptibility to developing | functional markers of T cells |
| adverse events, or that can lead to improved | Longitudinal changes in phenotypic and |
| adverse event monitoring or investigation | functional markers in peripheral blood |
| (i.e., safety biomarkers), or can provide | mononuclear cells (PBMCs) including but |
| evidence of mosunetuzumab activity (i.e., | not limited to B cell or other immune cell |
| pharmacodynamic (PD) biomarkers), or can | subset profiling (e.g., naïve, active, and |
| increase the knowledge and understanding of | memory cells) and/or immune cell |
| disease biology and drug safety or PK | production of cytokines or other products |
| | Longitudinal changes in CRS-associated |
| | biomarkers (e.g., interleukin 6 (IL-6) and |
| | interleukin 10 (IL-10)) |
| | Longitudinal changes in immunoglobulins |
| | and vaccination-related antibodies |
| | Proportion of patients achieving B cell |
| | counts below the limit of detection by flow |
| | cytometry at every visit |
| | Duration of B cell depletion |
| | Changes in number and phenotype of |
| | immune cells and/or stroma in biopsy |
| | tissue |
| To evaluate potential relationships between | Relationship between serum |
| drug exposure and biomarkers or | concentrations or PK parameters for |
| pharmacodynamics, efficacy, and safety of | mosunetuzumab and biomarker or |
| mosunetuzumab | pharmacodynamics, efficacy, and safety |
| | endpoints, as appropriate |
| To evaluate potential effects of ADAs on | Relationship between ADA status and |
| relevant endpoints | efficacy, safety, PK, or biomarker |
| | endpoints, as appropriate |

### C. Overall Design

### Description of Trial Design

This is a two-cohort, Phase II, multicenter, single-arm, open-label study designed to evaluate the efficacy, safety, and pharmacokinetics of mosunetuzumab in people with active lupus nephritis (LN; Cohort 1) and in people with active systemic lupus (Cohort 2). Participants in Cohort 1 may have extrarenal manifestations, but key enrollment criteria depend upon the existence of specific features of lupus nephritis and failure of that lupus nephritis to respond to prior specified therapies, as per enrollment criteria. Participants in Cohort 2 may have concomitant lupus nephritis, but key enrollment depends upon the existence of specific extrarenal manifestations and failure of those manifestations to respond to prior specified therapies, as per enrollment criteria. The assessments done depend not only upon whether participants are enrolled as part of Cohort 1 or Cohort 2 but also depend upon whether they have lupus nephritis at baseline or extrarenal manifestations at baseline.

The study begins with enrollment of Cohort 1 only and enrollment of Cohort 2 begins at the direction of the Sponsor. It is anticipated that Cohort 2 enrollment begins after approximately 10 to 15 participants have been enrolled into Cohort 1. Cohort 1 enrollment stops at the direction of the Sponsor and may or may not overlap with the simultaneous enrollment into Cohort 2.

Approximately 30 sites globally participate to enroll approximately 30 participants. Both cohorts are analyzed together for the primary endpoint of achievement of remission (e.g., drug free remission; cSLEDAI=0 after completion of mosunetuzumab treatment and off of lupus-directed therapies (with the exception of antimalarials) for 6 months) by Week 76.

After a screening period of up to 28 days (screening window is able to be extended by up to 7 days to permit completion of eligibility assessment following discussion with the Medical Monitor), enrolled participants receive subcutaneous injections of mosunetuzumab on Day 1 (1.6 mg), Day 8 (5 mg), Day 15 (45 mg), Day 22 (45 mg) and then 45 mg every 3 weeks for 7 more doses (for a total of 11 doses). Alternatively, the dosing schedule of mosunetuzumab can be described as a dosing regimen comprising nine 21-day dosing cycles, wherein the first dosing cycle comprises 3 doses, C1D1 (1.6 mg of mosunetuzumab), C1D2 (5 mg of mosunetuzumab), and C1D3 (45 mg of mosunetuzumab), administered on Days 1, 8, and 15, respectively, of the first dosing cycle, and each of the second to ninth dosing cycles comprises a single 45 mg dose of mosunetuzumab (C2D1-C9D1, respectively), administered on Day 1 of each respective dosing cycle.

Eligible participants for Cohort 1 are required to have active Class III or IV lupus nephritis (as defined by the 2018 International Society of Nephrology/Renal Pathology Society (ISN/RPS) Classification Guidelines (Bajema et al., Kidney International, 93(4):389-796, 2018)) demonstrated on a renal biopsy within 3 months of screening or during screening, Complement C3 level below the lower limit of normal at screening, and proteinuria (UPCR ≥1.5 g/g on a 24-hour urine collection at screening) despite treatment for at least 6 months with standard LN therapy besides corticosteroids. Participants with Class V membranous nephritis in addition to Class III or IV LN on renal biopsy are eligible. Participants of Cohort 1 who complete mosunetuzumab treatment and achieve both a clinical SLEDAI of zero and peripheral B-cell depletion undergo a repeat renal biopsy to help assess whether the participant's disease activity is sufficiently quiescent to safely discontinue their remaining background SLE immunosuppressants (e.g. MMF, azathioprine (AZA)). All Cohort 1 participants, regardless of their clinical status, must have a repeat kidney biopsy no later than Week 44.

Eligible participants for Cohort 2 are required to have SLE diagnosed at least 6 months prior to screening per 2019 European League Against Rheumatism (EULAR)/American College of Rheumatology (ACR) SLE Classification Criteria (Aringer et al., Arthritis Rheumatol, 71(9):1400-1412, 2019); anti-double stranded DNA antibody or anti-Smith antibody above the upper limit of normal at screening; Complement C3 level below the lower limit of normal at screening; active extrarenal disease at screening, defined by an extrarenal Systemic Lupus Erythematosus Disease Activity Index (SLEDAI)-2K score 28 (excluding points for any renal component as well as excluding points for the nonspecific manifestations of alopecia, headache, and fever); and a history of active disease despite treatment for at least 6 months with either cyclophosphamide or a biologic (e.g., belimumab, anifrolumab, rituximab or other anti-CD20 agent). Cohort 2 participants who, in addition to having extrarenal disease, have active lupus nephritis or significant proteinuria are encouraged to undergo a kidney biopsy at screening (if they have not had a kidney biopsy within 3 months of screening) and a repeat kidney biopsy after completion of mosunetuzumab treatment (prior to initiating the taper of background immunosuppressants such as MMF). Participants in Cohort 2 who complete mosunetuzumab treatment and achieve a clinical SLEDAI of zero and peripheral B-cell depletion, together with improvements in symptoms not included in the SLEDAI-2K, should begin to discontinue their SLE background immunosuppressants (e.g. MMF) except for antimalarials.

The timepoint when a participant is eligible to discontinue their SLE background immunosuppressants is known as the Background Immunosuppressant Discontinuation Eligibility Point or BDEP. BDEP may differ between participants because it is based on the participants meeting clinical criteria following completion of mosunetuzumab treatment. Oral corticosteroids (OCS) and calcineurin inhibitors (CNIs) should be tapered earlier in the study, within the mosunetuzumab treatment phase.

Study visits with clinical and laboratory assessments occur on all dosing days, and then continue every 2 months thereafter until Week 76, then every 3 months until Week 104 to assess achievement, maintenance and durability of remission.

Participants unable to completely discontinue background immunosuppression (including OCS) within the first 6-7 months continue to attempt tapering to eventual discontinuation throughout the duration of study participation.

Participants are considered to have achieved remission (e.g., drug-free remission) when they reach and maintain a cSLEDAI=0 while off of all SLE therapy (except for antimalarials) for 6 months. Given the heterogeneity of lupus, it is anticipated that different active manifestations may resolve along different timepoints. While some participants may achieve cSLEDAI=0 prior to completion of mosunetuzumab treatment, they must maintain a cSLEDAI=0 for 6 months following completion of mosunetuzumab treatment while not receiving any other SLE therapies besides antimalarials.

Safety Follow-Up (SFU): All participants who receive any amount of mosunetuzumab are followed for safety for at least 18 months from the last mosunetuzumab dose. Thus, for those whose last dose is administered on schedule by Week 24, participants are followed until at least Week 104 for safety monitoring. A participant who experiences treatment interruption(s) and dose delay(s) whereby the last dose of mosunetuzumab treatment is given by Week 52 requires SFU through at least Week 130, respectively.

If a participant's B cell count recovers to the lowest pre-treatment (either screening or baseline) value or the lower limit of normal (LLN), whichever is lower, by the time 18 months have elapsed since the last mosunetuzumab dose, no further SFU is required.

If a participant's B cell count does not recover to the lowest pre-treatment value or the LLN, whichever is lower, by the by the time 18 months have elapsed since the last mosunetuzumab dose, SFU continues every 6 months until any of the following occurs:
Return of peripheral CD19+ B cells to the lowest pre-treatment value or the LLN, whichever is lower
Receipt of therapies associated with reductions in peripheral B cells
The study ends If any therapy associated with reductions in peripheral B cells (including, but not limited to, rituximab, cyclophosphamide, obinutuzumab, ofatumumab, or belimumab) is planned during the SFU period after Week 104, a final SFU visit occurs prior to the receipt of this therapy.

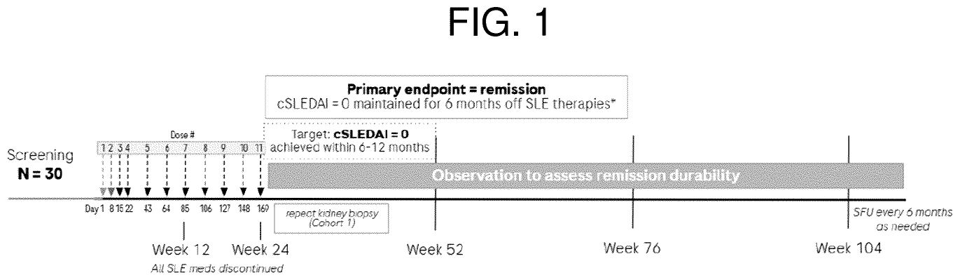

A study schema is provided in FIG. 1.

### Duration of Participation

The total duration of study participation for an individual is approximately 2 years. Participants who require dose delay(s) are able to extend participation for an additional 6 months. Participants who are required to participate in the Safety Follow-up (for monitoring B cell repletion) will remain in the study for at least 6 months longer or until study end.

### Rationale for Trial Design

### Rationale for Trial Population

Historically, efficacy of lupus therapies has been defined as achieving a meaningful reduction in, rather than elimination of, disease activity. This is attributed to numerous factors, including the heterogeneity, multi-organ involvement and relapsing-remitting nature of the disease, as well as limitations in the effect of any particular treatment, thus leading to a combinatorial approach involving most patients' treatment regimens comprising multiple immunomodulatory medicines.

Since B cell-directed chimeric antigen receptor T cell (CAR-T) therapies have been tested outside of the oncology setting in patients with autoimmune disease, including SLE with LN, elimination of disease activity with such an approach now appears possible (Mackensen et al., Nat Med, 28:2124-32, 2022 and Muller et al., N Engl J Med, 390:687-700, 2024). The remarkable efficacy of CAR-T therapy in lupus is attributed to the engagement of T cells to direct B cell killing, both in the peripheral circulation and especially at the tissue level. Similarly, a bispecific T cell-engager such as mosunetuzumab, which can be administered in the outpatient setting and without the aggressive chemotherapy pre-conditioning and prolonged hospitalization required for CAR-T administration, may confer a similar depth of B cell depletion and corresponding remission.

Participants with LN (Cohort 1) have poor prognosis and require intensive immunosuppressive regimens to achieve disease control. The Cohort 2 patient population (systemic lupus) is at increased risk for major organ involvement, death, and/or complications of treatment. Both patient populations, therefore, are appropriate for study in the proposed clinical trial. Autoantibody positivity and hypocomplementemia are associated with higher likelihood of severe disease, increased organ damage, and potentially poorer long-term outcomes (Hoi A et al., Lancet, 403:2326-38, 2024). A higher Systemic Lupus Erythematosus Disease Activity Index 2000 (SLEDAI-2K; Gladman et al., J. Reumatology, 29:288-91, 2002) score is associated with increased mortality and damage accrual (Cook et al., J. Reumatology, 27:1892-5, 2000; Gladman et al., J. Reumatology, 29:288-91, 2002; Nossent et al., Lupus, 19:949-56, 2010). Moderate to severe disease activity and immunosuppressant use is associated with increased mortality (Lopez et al., Rheumatology, 51:491-8, 2012).

In addition, this patient population appears to receive the greatest benefit from B cell targeted therapies, as evidenced by recent clinical trials of therapies targeting B cell survival factors. With belimumab, in a post-hoc analysis from pooled data from two pivotal trials (van Vollenhoven et al., Ann Rheum Dis, 71:1343-9, 2012), and a pre-specified analysis from a subsequent pivotal trial (Stohl et al., Arthritis Rheumatol, 69:1016-27, 2017), the greatest degree of clinical benefit on disease activity, risk of subsequent flare, and patient-reported outcomes (PROs) over standard of care alone was observed among patients with high baseline disease activity and those with serologic activity, as evidenced by the presence of anti-double stranded DNA (dsDNA) antibodies and/or hypocomplementemia. Similar findings were seen in subgroup analyses from a Phase II study of atacicept for the treatment of SLE (Merrill et al., Arthritis Rheumatol, 70:266-76, 2018) and Sponsor analyses of rituximab and fenebrutinib data (unpublished).

### D. Trial Population

### Description of Trial Population

Approximately 30 individuals total with either active lupus nephritis (Cohort 1) or active systemic lupus (Cohort 2) are enrolled in this study. The study begins with enrollment of Cohort 1 only and enrollment of Cohort 2 begins at the direction of the Sponsor. It is anticipated that Cohort 2 enrollment may begin after approximately 10 to 15 participants are enrolled into Cohort 1. Cohort 1 enrollment stops at the direction of the Sponsor and may or may not overlap with the simultaneous enrollment into Cohort 2.

Prospective approval of protocol deviations to recruitment and enrollment criteria, also known as protocol waivers or exemptions, is not permitted.

### Inclusion Criteria

Potential participants are eligible to be included in the study only if all of the following criteria apply:
Signed Informed Consent Form
Ability and willingness to comply with all aspects of the protocol including completion of procedures, interviews, questionnaires, assessments for the duration of the study
Agreement to adhere to the contraception requirements
Age ≥18 years at the time of signing Informed Consent Form
Diagnosis of SLE for 26 months as assessed using the 2019 European League Against Rheumatism/American College of Rheumatology (EULAR/ACR) Classification Criteria at screening

### Cohort 1

Active Class III and/or Class IV LN per 2018 ISN/RPS Criteria as confirmed through local laboratory testing of a renal biopsy sample obtained within 3 months of screening, which must meet all of the following criteria:
   ≥3 points on the National Institutes of Health (NIH) activity index, excluding any contribution from interstitial activity
   <50% glomerulosclerosis and <50% tubulointerstitial fibrosis
   Concomitant Class V disease is permitted
   A qualified nephropathologist must verify that participants meet enrollment criteria based on their kidney biopsy. If a qualified nephropathologist is not available at the local site level, a central nephropathologist may evaluate kidney biopsies to verify that the patient has met enrollment criteria.
Participants with biopsy-proven active LN as described above must also meet the following criteria for enrollment:
   UPCR ≥1.5 g/g on a 24-hour urine collection at screening per the central laboratory
   Complement C3 below the lower limit of normal at screening per the central laboratory
   Active LN despite treatment for at least 6 months with standard nonsteroidal LN therapy (e.g., MMF, cyclophosphamide, azathioprine, voclosporin, other calcineurin inhibitors, belimumab, rituximab)
   Participants previously treated with B cell-depleting therapy (e.g., rituximab) are eligible to participate as long as the last treatment was given more than 6 months prior

### Cohort 2

Participants without LN and/or with SLE must have autoantibody positivity, hypocomplementemia, and high disease activity at screening despite treatment with advanced therapy as follows:
Either an anti-double-stranded DNA antibody or anti-Smith antibody above the upper limit of normal at screening per the central laboratory
Complement C3 level below the lower limit of normal at screening per the central laboratory
Active extrarenal disease at screening, defined as the following:
   Extrarenal SLEDAI-2K score ≥8 (not including points for proteinuria, pyuria, hematuria, casts; points for alopecia, fever, and headache are also excluded given the nonspecific nature of these features)
      Points for rash may only be given if Cutaneous Lupus Erythematosus Disease Area and Severity (CLASI; Albrecht et al., J Invest Dermatol. 125(5):889-94, 2005) activity score is ≥6
      Points for mucosal ulcers may only be given if they are significantly interfering with oral intake and clinical assessment confirms extensive and deep ulceration
      Points for arthritis may only be given if ≥4 joints are both swollen and tender and ≥2 are located in the wrist or hand
Active disease despite treatment for at least 6 months with either cyclophosphamide or a biologic (e.g., belimumab, anifrolumab, rituximab or other anti-CD20 agent)
   Participants previously treated with B cell-depleting therapy (e.g., rituximab) are eligible to participate as long as the last treatment was given more than 6 months prior

### Exclusion Criteria

Potential participants are excluded from the study if any of the following criteria apply:
Pregnant or breastfeeding, or intention of becoming pregnant during the study or within the time frame in which contraception is required
Participants of childbearing potential must have a negative serum pregnancy test result at screening prior to initiation of study treatment.
Treatment with investigational therapy within 30 days or 5 drug-elimination half-lives (whichever is longer) prior to initiation of study treatment and during the study
Major surgery requiring hospitalization during the 4 weeks prior to screening or during screening, or any planned surgery or procedure requiring hospitalization during the 12 weeks following study drug administration
Alcohol or substance abuse within the 12 months prior to screening
Active infection of any kind, excluding fungal infection of the nail beds
Any major episode of infection that fulfills any of the following criteria:
   Requires hospitalization during the 8 weeks prior to screening or during screening
   Requires treatment with IV antibiotics (or anti-infective medications) during the 8 weeks prior to screening or during screening
   Requires treatment with oral antibiotics (or anti-infective medications) during the 2 weeks prior to screening or during screening
      Antibiotics or anti-infective medications given in the absence of a major episode of infection are not exclusionary.
History of serious recurrent or chronic infection
History of progressive multifocal leukoencephalopathy (PML)
Tuberculosis (TB) infection
   Testing for TB is performed at screening. The choice of test (skin or blood test) will be in accordance with local clinical practice.
      Latent TB after completion of appropriate treatment is not exclusionary.
Positive human immunodeficiency virus (HIV) (HIV-1 and HIV-2 antibody test) at screening
Positive hepatitis B surface antigen (HBsAg) and total anti-HBc test at screening
Positive hepatitis C virus (HCV) antibody test at screening
Known or suspected chronic active Epstein-Barr virus (EBV) infection or cytomegalovirus (CMV) infection at screening
History of cancer, including solid tumors, hematological malignancies, and carcinoma in situ, within the past 5 years
   Participants with non-melanomatous carcinomas of the skin that have been treated or excised and have resolved are eligible.
Any serious, significant or uncontrolled medical condition or abnormality in clinical laboratory tests that precludes an individual's safe participation in and completion of the study
Active overlap syndrome with mixed connective tissue disease or systemic sclerosis within the 12 months prior to screening or during screening
Catastrophic or severe antiphospholipid syndrome within the 12 months prior to screening or during screening. Antiphospholipid syndrome adequately controlled by anticoagulant therapy for at least 2 months prior to screening is acceptable.
High risk for clinically significant bleeding or any condition requiring plasmapheresis, IV immunoglobulin, or acute blood product transfusions
Active severe or unstable lupus-associated neuropsychiatric disease or where, in the opinion of the investigator, it is likely to require treatment with protocol-disallowed therapies. Examples of neuropsychiatric SLE manifestations include, but are not limited to the following: meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, and mononeuritis multiplex.
Significant or uncontrolled medical disease that precludes participant participation including but not limited to significant active cardiac disease (e.g., congestive heart failure, pericarditis and/or myocarditis) and/or active pulmonary disease (e.g., bronchospasm and/or obstructive pulmonary disease).
Glomerular filtration rate <30 mL/min/1.73 m² as calculated through use of the Chronic Kidney Disease Epidemiology Collaboration equation
History of any non-SLE disease treated with oral, intravenous, or intramuscular corticosteroids for more than 14 days in total during the one year prior to Day 1
History of treatment with any T cell-engaging bispecific antibodies or CAR-T therapy within the past 2 years
Receipt of any biologic therapy (other than anti-CD20, anti-CD19 or anti-plasma cell) such as, but not limited to, anifrolumab and/or belimumab, within 28 days of enrollment
Receipt of an anti-CD20 (e.g. rituximab, obinutuzumab), anti-CD19 (e.g. rituximab, obinutuzumab), or anti-plasma cell monoclonal antibody within 6 months of screening
Receipt of herbal therapies within 28 days of enrollment
Intolerance or contraindication to study therapies including history of severe allergic or anaphylactic reactions to monoclonal antibodies or known hypersensitivity to any component of mosunetuzumab injection
Receipt of cyclophosphamide within 12 weeks of screening.
Receipt of any live or attenuated vaccine in the 28 days prior to or during screening
Any of the following laboratory abnormalities:
   Aspartate transferase (AST) or alanine transaminase (ALT) >2.5× upper limit of normal (ULN)
   Total bilirubin >1.5×ULN

Participants with a documented history of Gilbert syndrome and in whom total bilirubin elevations are accompanied by elevated indirect bilirubin are eligible.
Platelet count <75×10⁹/L
Hemoglobin <90 g/L
Absolute neutrophil count (ANC)<1.5×10⁹/L (1500/μL)

### Screen Failure and Re-Screening

Individuals who do not meet the eligibility criteria for participation in this study (screen failure) may qualify for 3 re-screening opportunities (for a total of 4 screenings per individual) at the investigator's discretion. Re-screening is not permitted for individuals who do not meet the UPCR threshold of ≥1.5 g/g on a 24-hour urine collection. Individuals must re-sign the consent form prior to re-screening. The investigator maintains a record of reasons for screen failure.

### E. Trial Intervention and Concomitant Therapy

### Investigational Trial Intervention Administration

### Mosunetuzumab

Administration of study treatments is performed in a monitored setting with immediate access to trained critical care personnel and facilities, and adequate equipment to respond to and manage potentially serious reactions and medical emergencies.

Subcutaneous (SC) mosunetuzumab is administered to participants by SC injection using standard medical syringe with a final volume not to exceed 2.0 mL. Compatibility testing has shown that mosunetuzumab is stable in polypropylene syringes.

Mosunetuzumab is administered to well-hydrated participants. Flat dosing independent of body weight will be used for mosunetuzumab.

The dosing schedule of SC mosunetuzumab is 1.6 mg on Day 1, 5 mg on Day 8, 45 mg on Day 15, 45 mg on Day 22, and 45 mg every three weeks thereafter until all 11 doses have been administered. Alternatively, the dosing schedule of SC mosunetuzumab can be described as a dosing regimen comprising nine 21-day dosing cycles, wherein the first dosing cycle comprises 3 doses, C1D1 (1.6 mg), C1D2 (5 mg), and C1D3 (45 mg), administered on Days 1, 8, and 15, respectively, of the first dosing cycle, and each of the second to ninth dosing cycles comprises a single 45 mg dose (C2D1-C9D1, respectively), administered on Day 1 of each respective dosing cycle.

Vital signs are assessed prior to each mosunetuzumab injection (within 30 minutes prior to injection) and after administration of study drug.

Mosunetuzumab is administered by qualified staff over 30 seconds to 2 minutes.

### Premedications

The following premedications are administered for each mosunetuzumab injection in all participants:
Corticosteroid premedication consisting of 20 mg dexamethasone is administered orally prior to each of the first four mosunetuzumab injections, i.e., on Days 1, 8, 15, and 22. Dexamethasone premedication for Dose 5 and subsequent doses is optional if no CRS occurred with previous doses (if CRS occurred with the previous dose, dexamethasone premedication should be given before the next mosunetuzumab dose). Oral corticosteroid (OCS) dose should be held on the days when dexamethasone is administered.
Acetaminophen/paracetamol 650-1000 mg is administered orally prior to mosunetuzumab injection for the first four doses, and then is optional for subsequent doses.
Diphenhydramine 25-50 mg is administered orally prior to mosunetuzumab injection for the first four doses, and then is optional for subsequent doses.

### Concomitant Therapy

Any medication including over-the-counter or prescription medicines, vaccines, vitamins, and/or herbal supplements)} used by a participant from 7 days prior to initiation of study treatment to the last study visit is recorded on the Concomitant Medications eCRF along with the following information:
Reason for use
Dates of administration, including start and end dates
Dosage information, including dose and frequency

The Medical Monitor is consulted if there are any questions related to concomitant or prior therapy.

### Prohibited Concomitant Therapy

Use of the following concomitant therapies is prohibited as described below:
Investigational therapy (other than protocol-mandated study treatment) within 30 days or 5 drug-elimination half-lives (whichever is longer) prior to initiation of study treatment and during the study
Cyclophosphamide
Biologic therapies (e.g., belimumab, anifrolumab, rituximab)
Live or attenuated vaccines
Herbal therapies

### Permitted Concomitant Therapy

In general, investigators may manage a participant's care (including preexisting conditions) through use of supportive therapies, as clinically indicated and per local standard practice, with the exception of prohibited therapies and taking into account cautionary therapies.
Corticosteroids:
Oral prednisone (or equivalent) up to 60 mg/day for entry into the study, with the exception that the OCS dose should be held on the days when dexamethasone is administered as a premedication.

Tapering of OCS should be attempted in all participants unless clinically contraindicated, with a goal of prednisone (or equivalent) 55 mg/day by Week 12 and completely discontinued by Week 24.
Use of a single topical corticosteroid agent (with or without OCS) is permitted
Doses of prednisone (or equivalent) 55 mg/day to treat secondary adrenal insufficiency are permitted as long as laboratory testing confirms this diagnosis prior to initiation of such treatment (morning cortisol and Adrenocorticotropic Hormone (ACTH) levels below lower limit of normal (about 5 μg/dL for morning cortisol and about 7.2 pg/mL for ACTH) for the testing laboratory)

Participants who receive a limited course of corticosteroids alone (as described above) during the first 6 months of the study are not required to discontinue mosunetuzumab.
Antimalarial agents (hydroxychloroquine, chloroquine, or quinacrine) are permitted throughout participation in the study
Conventional immunosuppressants (i.e., azathioprine, methotrexate, MMF, calcineurin inhibitors, cyclophosphamide) are permitted during screening and following enrollment, but participants completely discontinue conventional immunosuppressants and OCS within the first 6 months of study participation.
Oral contraceptives with a failure rate of <1% per year
Hormone-replacement therapy
Non-steroidal anti-inflammatory drugs (NSAIDs) at approved dosage at the discretion of the investigator
Acetaminophen (paracetamol)
   Care must be taken to ensure the total dose of acetaminophen (paracetamol), including that given for premedication, does not exceed the approved daily dose range.
Calcium and/or vitamin D supplementation
Anticoagulant or anti-platelet therapy for control of thrombotic risk if started at least 2 months prior to screening
Antihypertensive therapy
   Antihypertensive therapy, including angiotensin-converting enzyme (ACE) inhibitors or angiotensin-receptor blockers (ARB), is permitted although initiation of new agents from screening until completion of the study is avoided if possible; combination therapy with ACE inhibitor and ARB is not permitted
Hydroxymethylglutaryl-CoA (HMG-CoA) reductase inhibitors (“statins”) and bisphosphonates HMG-CoA reductase inhibitors and bisphosphonates are permitted but may not be initiated until completion of the study unless clinically indicated per investigator discretion. If participants enter the study taking such medications, every effort should be made to keep their dosage stable to prevent confounding of study results.
Treatment of CRS according to published recommendations and/or institutional practice
Treatment of hemophagocytic lymphohistiocytosis (HLH) according to published recommendations and/or institutional practice
Treatment of ICANS according to published recommendations and/or institutional practice
Hematopoietic growth factors and blood component transfusion are not initiated during screening. After initiation of study drug, treatment of neutropenia, thrombocytopenia, and anemia is permitted and is instituted per institutional practice, the published guidelines, institutional practice and/or investigator preference based on individual participant risk fact.
Anti-infective prophylaxis for viral, fungal, bacterial, or Pneumocystis infections is permitted and should be instituted per institutional practice or investigator preference based on individual participant risk factors.
Vaccinations with non-live, non-attenuated vaccines (such as COVID-19)
   Concomitant administration of approved non-live Coronavirus disease 2019 (COVID-19) vaccines (mRNA, inactivated virus, and replication-deficient viral vector vaccines), including booster vaccines for novel variants, are strongly recommended for all patients. Information on COVID-19 vaccine administration is collected even if it was given more than 7 days prior to initiation of study treatment.
      The decision of whether and when to administer a COVID-19 vaccine is individualized by the Investigator in consultation with the participant and should follow local practice guidelines for the timing of vaccination administration prior to initiating therapy to maximize potential therapeutic benefit.
      Factors to consider by investigators when making individualized decisions for patients receiving mosunetuzumab:
         General condition of the patient and severity/seriousness of underlying disease
         Potential risks associated with SARS-CoV-2 infection and potential benefits and risks from COVID-19 vaccination
         Epidemiology of SARS-CoV-2 infection in the patient's location
      COVID-19 vaccines and other permitted vaccines are administered to participants, with the aim to complete the vaccination course at least 1 week prior to starting study treatment with mosunetuzumab.
      If COVID-19 vaccines are administered during study treatment, vaccination should be administered at least one week after the Day 22 mosunetuzumab 45 mg dose is completed or at least one week prior to any subsequent 45-mg mosunetuzumab dose.
In the event of new onset of SARS-CoV-2 infection during study treatment, appropriate therapy including, but not limited to, antivirals and/or monoclonal antibodies in accordance with local institutional guidance (El Chaer et al., Blood, 140:673-84, 2022) is recommended.

In general, investigators manage a patient's care with supportive therapies as clinically indicated, per local standard practice.

### Concomitant Therapy Tapering and Discontinuation

At each visit, tapering and/or discontinuation of immunosuppressive therapies, including OCS, is attempted in all participants when the following criteria are met:
Clinical stability or improvement compared with the prior visit (e.g., same or lower SLEDAI-2K score)
No active lupus manifestations that threaten permanent damage (e.g., CNS, cardiopulmonary, vasculitis, gastrointestinal, active discoid skin lesions) and/or prevent safe reduction in immunosuppressant dose level

### Cautionary Therapy

### Immunizations

The efficacy and safety of immunization during periods of peripheral B cell depletion have not been adequately studied. It is recommended that a patient's vaccination record and the need for immunization be carefully evaluated prior to receiving mosunetuzumab. For participants who are likely to require immunization in the foreseeable future, such as participants planning to travel to countries where specific immunization is required or participants requiring a vaccination/booster for their professional activity, any required vaccination/booster is given at least 28 days prior to receipt of study drug. Review of a patient's immunization status and/or need for the following vaccinations in advance of receipt of study drug is strongly recommended: tetanus; diphtheria; influenza; pneumococcus polysaccharide; varicella; measles, mumps, and rubella; and hepatitis vaccines.

The safety and efficacy of immunization with a live or attenuated live vaccine in B cell-depleted participants are not known. For this reason, the use of live or attenuated vaccines (e.g., measles, mumps, rubella, oral polio vaccine, Bacillus Calmette-Guerin, typhoid, yellow fever, vaccinia, or any other vaccines not yet licensed but belonging to this category) is specifically excluded for 28 days prior to screening through the end of study participation.

Vaccines that do not contain live organisms (e.g., influenza, Pneumovax®, tetanus, and SARS-CoV-2 vaccines) are not prohibited; however, vaccines received during peripheral B cell depletion may be less effective.

Medications Given with Precaution due to Effects Related to CYP Enzymes

Given the expected pharmacology of mosunetuzumab, the transient release of cytokines may suppress Cytochrome P450 (CYP450) enzymes and cause drug-drug interactions. Preliminary clinical data in patients with SLE suggest that SC mosunetuzumab administration induced a transient elevation in plasma IL-6 levels, with peak levels occurring in the majority of patients within 1-2 days of dosing and returning to baseline by 3-4 days. Participants at the highest risk of a drug-drug interaction are those receiving concomitant medications that are sensitive CYP450 substrates and have a narrow therapeutic index. Such concomitant medications should be monitored for toxicity and dose adjusted accordingly.

CYP450 enzymes in the liver are down-regulated by infection and inflammatory stimuli, including cytokines such as IL-6. Inhibition of IL-6 signaling in participants who are treated with tocilizumab may restore CYP450 activities to higher levels than those in participants not treated with tocilizumab, leading to increased metabolism of drugs that are CYP450 substrates. In vitro studies showed that tocilizumab has the potential to affect expression of multiple CYP enzymes, including CYP1A2, CY2B6, CYP2C9, CYP2C19, CYP2D6, and CYP3A4. The effects of tocilizumab on CYP2C8 or transporters are unknown.

The effect of tocilizumab on CYP enzymes may be clinically relevant for CYP450 substrates with narrow therapeutic index, where the dose is individually adjusted:
Upon initiation or discontinuation of tocilizumab in participants being treated with these types of medicinal products, therapeutic monitoring of effect (e.g., warfarin) or drug concentration (e.g., cyclosporine or theophylline) is performed and the individual dose of the medicinal product adjusted as needed.
Prescribers exercise caution when tocilizumab is co-administered with CYP3A4 substrate drugs for which a decrease in effectiveness is undesirable (e.g., oral contraceptives, lovastatin, atorvastatin).

The effect of tocilizumab on CYP450 enzyme activity may persist for several weeks after stopping therapy.

### Herbal Therapies

Concomitant use of herbal therapies is prohibited throughout the study because their pharmacokinetics, safety profiles, and potential drug-drug interactions are generally unknown.

### F. Trial Assessments and Procedures

### Trial Assessments and Procedures Considerations

### Optional Lymphoid Tissue Biopsy (Participants Providing Separate Consent)

Consenting participants undergo optional lymphoid tissue biopsies before study treatment initiation and may undergo additional on-treatment lymphoid tissue biopsies at any other time at the investigator's discretion (if deemed clinically feasible by the investigator). Samples collected via resection, core-needle biopsy (at least three cores preferred), or excisional, incisional, punch, or forceps biopsy are preferred.

Samples may be used for exploratory biomarker research.

### Repeat Renal Biopsy

Participants in Cohort 1 undergo a repeat renal biopsy following completion of mosunetuzumab treatment (between Weeks 24-28 for those who received mosunetuzumab according to the dosing regimen) in order to assess for evidence of disease activity or remission beyond the laboratory-based response measurements.

### Efficacy Assessments and Procedures

### Response Evaluation

Objective response is determined by the investigator at specified timepoints and is used to support decision-making with regard to background medication management (e.g., if immunosuppressive therapies can be tapered and discontinued). Assessments are performed by the same individual, if possible, to ensure internal consistency across visits.

SLEDAI-2K, CLASI, and tender/swollen joint counts (TSJC) comprise the core SLE clinical disease activity assessments used to evaluate efficacy. Therefore, it is critical that these data are reported consistently and accurately.

Investigators managing participants in Cohort 1 only complete SLEDAI-2K at study visits. Investigators managing participants in Cohort 2 complete SLEDAI-2K in addition to CLASI and TSJC at study visits.

SLEDAI-2K scores for participants screened for participation in Cohort 2 are reviewed by the Sponsor or designee prior to enrollment.

Each SLE disease activity assessment requires investigator judgment to determine if the manifestation is present and attributable to active lupus. Therefore, these assessments are only completed by study investigators who have completed disease assessment training and certification. It is strongly recommended that the same clinician assessor perform all longitudinal disease activity assessments on an individual participant.

All study personnel receive a protocol-specific SLE Disease Activity Assessments Guide that provides detailed guidance on the use of these instruments in this study, including study-specific clarifications and extensions to ensure consistency and accuracy. Data from these instruments are regularly reviewed to ensure quality.

Endpoints (e.g., remission, response, complete and partial renal response for participants with active LN) are calculated programmatically by the Sponsor on the basis of investigator assessments of response at each specified timepoint.

### SLEDAI-2K

SLEDAI-2K is a valid, reliable, and sensitive instrument for assessing global disease activity. SLEDAI-2K contains 24 lupus-related items/descriptors that are marked as “present” if they are most likely attributable to active lupus, or as “absent” in the participant within the past month. Each item has a fixed number of points based on general clinical meaningfulness (range: 1-8) with a maximum possible score of 105. The SLEDAI-2K is valid for assessment of lupus-related items within the past 10 days and the past 30 days (Touma et al., Lupus, 20:67-70, 2011).

A key principle for SLEDAI-2K scoring is attribution: SLEDAI-2K items are only scored if the feature is most likely attributable to active SLE rather than alternative causes or chronic damage alone. By indicating that an item is present, the clinician assessor believes the feature represents an active, inflammatory manifestation of SLE; for example, hematuria due to active lupus nephritis. If any SLEDAI-2K item is marked as “absent” due to an alternative non-SLE explanation (e.g., fever due to infection, thrombocytopenia due to drug cause), this alternative explanation is documented.

SLEDAI-2K scoring follows the instrument scoring instructions with protocol-specific clarifications (Table 7). Laboratory-based items reflect the central laboratory results obtained from the same visit for which SLEDAI-2K is completed (e.g., results from samples collected at the Week 24 visit should be used to complete the Week 24 SLEDAI-2K rather than using Week 21 laboratory results).

TABLE 7
Protocol-Specific Clarifications for SLEDAI-2K Items

| SLEDAI-2K Item | Protocol-Specific Clarification |
|---|---|
| Lupus headache | True lupus headache is very rare. |
| | It is considered a severe manifestation of |
| | neuropsychiatric lupus and should prompt |
| | urgent diagnostic evaluation (e.g., with |
| | lumbar puncture, neuroimaging, evaluation |
| | by a neurologist) and treatment. |
| | Do not record isolated migraine, tension, or |
| | cluster headache. |
| Arthritis | Arthritis requires tenderness to palpation |
| | plus at least one additional sign of |
| | inflammation: swelling, effusion, warmth, or |
| | erythema. |
| | Do not record isolated tenderness to |
| | palpation. |
| Hematuria | Hematuria is attributed to active lupus |
| | nephritis and other causes are excluded. In |
| | the absence of proteinuria, attribution to |
| | active nephritis is very unlikely. |
| | Do not record hematuria associated with |
| | menstruation. The urine sample is repeated |
| | after menstruation. |
| Proteinuria | Exclude stone, infection, or an alternative |
| | cause. |
| Pyuria | Pyuria is attributed to active lupus nephritis |
| | and other causes are excluded. In the |
| | absence of proteinuria, attribution to active |
| | nephritis is very unlikely. |
| | Do not record pyuria associated with |
| | vaginal contamination (presence of any |
| | squamous epithelial cells) or infection. |
| Rash | Do not record damage (e.g., chronic |
| | scarred discoid lesions). |
| | A cutaneous lesion can be scored as “rash” |
| | or “vasculitis”, but not both. If cutaneous |
| | vasculitis is present and SLEDAI-2K |
| | vasculitis is scored, rash can only be scored |
| | if there is an additional active, non-vasculitic |
| | rash present. |
| Alopecia | Must have ongoing hair loss without |
| | scarring. |
| | Do not record alopecia areata, androgenic |
| | alopecia, or chronic (inactive) temporal |
| | thinning. |
| Mucosal ulcers | In most cases, oral ulcers are painless and |
| | are located on the posterior hard palate. |
| | These may relapse and remit with SLE |
| | disease activity in other domains. |
| | Non-ulcerative lesions of the mouth or nose |
| | (including isolated erythema) must not be |
| | scored. |
| | Recurrent aphthous stomatitis (“canker |
| | sores”) lesions are painful, circular or ovoid, |
| | and have an erythematous rim. These are |
| | not scored. |
| Increased DNA | Anti-double stranded DNA (dsDNA) |
| binding | antibody above the upper limit of normal |
| | (including indeterminate or equivocal) at the |
| | central laboratory. |
| Leukopenia | Lymphopenia without leukopenia is not |
| | scored. |

### CLASI

CLASI is a validated instrument for assessing cutaneous lupus activity. It reports two global scores: one for current inflammatory activity (Total Activity Score) and one for damage (Total Damage Score).

To calculate the total scores described above, trained assessors evaluate erythema, scale/hypertrophy, dyspigmentation, and scarring/atrophy/panniculitis at 13 anatomic locations plus additional assessments for mucosal lesions and alopecia.

CLASI scoring must be consistent with corresponding items in SLEDAI-2K (rash, alopecia, mucosal ulcers, vasculitis).

### Joint Counts

A 28-joint count of tender and swollen joints (TSJC) is performed at each visit. The following joints on left and right sides are examined:
Metacarpophalangeal 1-5
Proximal interphalangeal 1-5
Shoulder, elbow, wrist, knee

Each joint is evaluated separately for tenderness (via palpation) and swelling. Joints with intra-articular injection within the preceding 4 weeks are excluded.

TSJC scoring must be consistent with the corresponding item in SLEDAI-2K (arthritis).

### Training and Certification for SLE Disease Activity Assessments

The Sponsor or its representatives provide comprehensive SLE disease activity assessment training. All assessors complete this training and pass an examination before assessing or enrolling participants.

### Efficacy Clinical Outcome Assessments

Clinician-reported outcome (ClinRO) and Patient-reported outcome (PRO) instruments are completed to assess the treatment benefit of mosunetuzumab.

ClinRO data are collected through the use of the SLEDAI-2K, CLASI, and TSJC instruments. Participants who achieve a cSLEDAI-2K score of 0 after completion of mosunetuzumab treatment and maintain this for at least 6 months off of all SLE therapies are considered to have achieved remission.

ClinRO instruments are completed at the clinic at specified timepoints during the study. The instruments are provided by the Sponsor in pre-printed booklets. The clinician investigator or sub-investigator serves as the clinical assessor and complete the official version of each ClinRO instrument, as provided by the Sponsor. Instruments are not copied from the protocol.

All paper-based ClinRO entries are transcribed into a corresponding eCRF for Sponsor review and oversight.

Participant-reported outcome (PRO) data is collected through the use of the following paper-based instruments:
Functional Assessment of Chronic Illness Therapy (FACIT)-Fatigue Scale (Kosinski et al., Lupus, 22(5):422-30, 2013)
Subject's Global Assessment of Disease Activity (SGA), as described in Scott P J, Huskisson E C. Rheumatol. Rehabil. 16(4):257-9, 1977.

### Safety Assessments and Procedures

Participants are closely monitored for safety throughout the study. Any abnormality (e.g., physical examinations, ECG, vital signs) identified at screening or prior to initiation of study treatment should be recorded as a preexisting condition. In addition, during this time period, events considered serious and related to a protocol-mandated procedure are collected and reported as serious adverse events.

### Pharmacokinetics

Serum samples are used to evaluate the pharmacokinetics of mosunetuzumab through use of a validated assay method by or under the supervision of the Sponsor.

PK samples are destroyed no later than 5 years after the final Clinical Study Report has been completed to allow for assay development and validation and exploratory research (if needed).

After PK analyses are completed, any remaining serum may be used for exploratory biomarker research.

### Biomarkers

The following biomarker samples are collected, as applicable, from participants at all sites:
Flow cytometry:
   T cells (CD3, CD4, CD8), B cells (CD19), and NK cells (CD16, CD56) as determined on a T, B, and NK cell (TBNK) assay
   CD19+ B cells and B cell subset panel by means of high-sensitivity flow cytometry (HSFC) (lower limit of quantification: 0.441 cells/μL)
   T cell phenotyping panel including, but not limited to Ki67, CD69, and TIM3, was performed using conventional flow cytometry
Serum and plasma for CRS-associated biomarkers: potentially including, but not limited to IL-6, IL-10, IL-8, IFN-g, and TNF-a
Blood, peripheral blood mononuclear cells (PBMCs), urine, plasma, and serum samples for exploratory research on biomarkers and biomarker assay development
Archival or newly collected kidney tissue samples or lymph node samples for exploratory research on biomarkers

Biomarker samples are collected. Biomarker samples are sent to one or several central laboratories or to the Sponsor or a designee.

Exploratory biomarker research may include, but is not limited to, cytokines associated with B cell function and activation (e.g., soluble B cell-activating factor (sBAFF), and B cell maturation antigen (BCMA), autoantibodies associated with SLE, and T cell subsets (e.g., activation and proliferation). Exploratory biomarkers in the urine may additionally include, but are not limited to, proteins associated with kidney cell damage and immune cells. Research may involve extraction of DNA and RNA for analysis of non-inherited BCR and TCR sequences and genomic profiling through use of NGS of a comprehensive panel of genes. Genomic profiling may include whole genome sequencing (WGS) or whole exome sequencing (WES) of blood samples. Exploratory research may also assess changes in cells and biomarkers with LDC and rimiducid treatment.

On the basis of emerging data, the number of biomarker samples may be reduced, or sample collection may cease altogether. Additionally, testing of collected samples may be halted, if warranted. Any decision to stop sample collection and testing is documented.

Unless the participant gives specific consent for any remaining blood, plasma, serum, urine, PBMC and biopsy tissue to be stored for optional exploratory research, biomarker samples are destroyed no later than 10 years after the final Clinical Study Report is completed. The storage period is in accordance with the Institutional Review Board or Ethics Committee (IRB/EC)-approved Informed Consent Form and applicable laws (e.g., health authority requirements).

For enrolled participants, remaining archival tissue blocks are returned to the site upon request or no later than completion of the final Clinical Study Report, whichever occurs first. For individuals who are not enrolled, remaining archival tissue blocks are returned to the site no later than 6 weeks after eligibility determination.

The storage period is in accordance with applicable laws (e.g., health authority requirements) and the Informed Consent Form approved by the Institutional Review Board or Ethics Committee (IRB/EC).

Given the complexity and exploratory nature of exploratory biomarker analyses, data derived from these analyses are not generally provided to study investigators or participants unless required by law. The aggregate results of any conducted research will be available in accordance with the effective Sponsor policy on study data publication.

### Blood Samples for Whole Genome Sequencing or Whole Exome Sequencing (Participants at Participating Sites)

At participating sites, blood samples are collected for DNA extraction to enable WGS or WES to identify variants that are predictive of response to study treatment, are associated with progression to a more severe disease state, are associated with susceptibility to developing adverse events, can lead to improved adverse event monitoring or investigation, or can increase the knowledge and understanding of disease biology and drug safety. Research includes exploration of germline variants.

The samples may be sent to one or more laboratories for analysis.

Collection and submission of blood samples for WGS or WES is contingent upon the review and approval of the exploratory research by each site's IRB/EC and, if applicable, an appropriate regulatory body. If a site has not been granted approval for WGS or WES, this section of the protocol is not applicable at that site.

Genomics is increasingly informing researchers' understanding of disease pathobiology. WGS and WES provide a comprehensive characterization of the genome and exome, respectively, and, along with clinical data collected in this study, may increase the opportunity for developing new therapeutic approaches or new methods for monitoring efficacy and safety or predicting which participants are more likely to respond to a drug or develop adverse events. Data are analyzed in the context of this study but may also be explored in aggregate with data from other studies. The availability of a larger dataset assists in identification and characterization of important biomarkers and pathways to support future drug development.

For sampling procedures, storage conditions, and shipment instructions, see the laboratory manual.

Blood samples collected for WGS or WES are to be stored until they are no longer needed or until they are exhausted. However, the storage period is in accordance with the IRB/EC-approved Informed Consent Form and applicable laws (e.g., health authority requirements).

Data generated from blood samples collected for WGS or WES are analyzed in aggregate rather than on an individual participant basis. Thus, there is no identification and reporting of incidental findings to investigators or participants.

The aggregate results of any conducted research are available in accordance with the effective Sponsor policy on study data publication.

### Immunogenicity Assessments

Antibodies to mosunetuzumab are evaluated in serum samples.

Serum samples are screened for antibodies binding to mosunetuzumab, and the titer of confirmed positive samples is reported. Other analyses may be performed to further characterize the immunogenicity of mosunetuzumab.

After immunogenicity analyses are completed, any remaining serum samples may be used for exploratory biomarker research.

### G. Statistical Considerations

### Statistical Hypotheses

The purpose of the study is hypothesis generation regarding the treatment effect of Mosunetuzumab on achievement of remission relative to the historical comparators. In order to estimate the response rate in remission, the rate of Definition of Remission in SLE (DORIS), which is a comparable endpoint, was used in the calculation below. The weighted average on the rate of DORIS remission based on historical randomized controlled trials is 9.2% (Table 8). Remission from historical trials was defined as achieving a clinical SLEDAI-2K=0 and a PGA <0.5, with permitted use of stably dosed antimalarials, low-dose glucocorticoids, and/or immunosuppressants, including biologics. Compared to DORIS remission, the primary endpoint defined in the current study is thought to be more restrictive due to the requirement of discontinuing SLE therapies and a 6-month sustained remission off SLE treatment. Therefore, the comparator rate of remission defined in the primary endpoint is assumed to be lower at 5%.

TABLE 8
DORIS Remission Rate in SLE Studies

| | | Timepoint for | | Number of | | |
|---|---|---|---|---|---|---|
| | | DORIS | Treatment | patients | Responders | |
| Molecule | Study | assessment | arm | treated | (n/%) | |
| Anifrolumab | TULIP- | W 52 | 300 mg | 360 | 55 | (15.3%) |
| | 1/TULIP-2 | | | | | |
| | pooled | | | | | |
| Anifrolumab | TULIP- | W 52 | PBO | 366 | 28 | (7.6%) |
| | 1/TULIP-2 | | | | | |
| | pooled | | | | | |
| Atacicept | ADDRESS II | W 24 | 75 mg | 55 | 8* | (14.5%) |
| Atacicept | ADDRESS II | W 24 | 150 mg | 51 | 7* | (13.7%) |
| Atacicept | ADDRESS II | W 24 | PBO | 52 | 2* | (3.8%) |
| Belimumab | BISS- | W 52 | all | 1684 | 142 | (8.4%) |
| | 52/BLISS-76 | | | | | |
| Belimumab | BLISS | W 64 | 200 mg + | 144 | 9 | (6.3%) |
| | BELIEVE | | RTX | | | |
| Belimumab | BLISS | W 64 | 200 mg + | 47 | 5 | (10.6%) |
| | BELIEVE | | SoC | | | |
| Belimumab | BLISS | W 64 | 200 mg + | 72 | 4 | (5.6%) |
| | BELIEVE | | PBO | | | |

*Number of responders were estimated based on the % provided in the manuscript.

### Statistical Analyses

The Statistical Analysis Plan (SAP) is finalized prior to database lock for the primary analysis, and it includes a more technical and detailed description of the statistical analyses described in this section. This section is a summary of the planned statistical analyses of the most important endpoints, including primary and key secondary endpoints. The analyses specified in the SAP supersede those specified here.

### General Considerations

Detailed analysis methods are described in the following sections. All efficacy endpoints are analyzed using the efficacy-evaluable set that includes all enrolled participants regardless of whether they received study treatment. Safety analysis is performed using safety-evaluable set.

For each participant, the first day of study treatment is designated as Study Day 1. Each assessment is assigned a study day calculated as follows:
Date of Assessment−Date of Treatment Day 1+1

Baseline is defined as the last non-missing value prior to the receipt of study treatment. Typically, baseline is the pre-dose Day 1 assessment, but may include screening results if the pre-dose Day 1 result is not available or missing.

### Estimation Methods for the Primary Endpoint

The primary estimand attributes are as follows:
Population: Participants with active SLE with or without LN as defined by the study inclusion and exclusion criteria
Variable (primary endpoint): Achievement of remission by Week 76. Remission is defined as achieving both of the following:
   Absence of disease activity (cSLEDAI=0) maintained for 6 months
   Not receiving any SLE-directed therapy (except for antimalarials) during the 6 months
Treatment:
   Mosunetuzumab SC: 1.6 mg Day 1, 5 mg Day 8, 45 mg Day 15, 45 mg Day 22 followed by 45 mg every 3 weeks until the final dose on Day 169
Intercurrent events and handling strategies:
   Death: composite strategy
   Major concomitant therapy violation: composite strategy
   Receipt of rescue therapy other than corticosteroids: composite strategy
   Use of pulsed steroids or sustained, high-dose oral corticosteroids after week 24: composite strategy
   Early treatment discontinuation: treatment policy strategy
Summary measure: Proportion of participants who achieved remission and the 95% CI is calculated using Clopper-Pearson method

The achievement of remission in Mosunetuzumab-treated patients is estimated using proportions and its 95% Clopper-Pearson CI. The lower bound of the CI is compared to the historical remission rate of 5% to test for the hypothesis.

Any missing or ambiguous data is queried, and all efforts are made to resolve data issues prior to the primary analysis snapshot and final database lock. Missing data in SLEDAI-2K questionnaire is imputed at item level using last observation carried forward method.

### Estimation Methods for the Secondary Endpoints

The secondary endpoints are defined in Table 5.

Estimand attributes and main analytical approaches are outlined below.

The estimand attributes for the binary secondary endpoints are defined as follows:
Population: Participants with active SLE with or without LN as defined by the study inclusion and exclusion criteria
Variable (endpoint): binary secondary endpoint of interest
Treatment:
   Mosunetuzumab SC: 1.6 mg Day 1, 5 mg Day 8, 45 mg Day 15, 45 mg Day 22 followed by 45 mg every 3 weeks until the final dose on Day 169
Intercurrent events and handling strategies:
   Death: composite strategy
   Receipt of rescue therapy: composite strategy
   Use of pulsed steroids or sustained, high-dose oral corticosteroids after week 24: composite strategy
   Major concomitant therapy violation: composite strategy
   Early treatment discontinuation: treatment policy strategy
Summary measure: Proportion of participants achieving response at a specific visit of interest and its confidence interval

Safety is assessed through summaries of exposure to study treatment, adverse events, changes in laboratory test results, and changes in vital signs and electrocardiograms (ECGs).

Study treatment exposure (such as treatment duration, total dose received, and number of cycles and dose modifications) are summarized with descriptive statistics.

All verbatim adverse event terms are mapped to medical dictionary for regulatory activities (MedDRA) thesaurus terms, and adverse event severity is graded according to NCI CTCAE v5.0. All adverse events, serious adverse events, adverse events leading to death, adverse events of special interest, and adverse events leading to study treatment discontinuation that occur on or after the first dose of study treatment (i.e., treatment-emergent adverse events) are summarized by mapped term, appropriate thesaurus level, and severity grade. For events of varying severity, the highest grade is used in the summaries. Deaths and cause of death are summarized.

### Pharmacokinetic Analyses

Individual and mean serum mosunetuzumab concentration versus time data are tabulated and plotted. The serum pharmacokinetics of mosunetuzumab are summarized by estimating relevant PK parameters (as appropriate for data collected). Estimates for these parameters are tabulated and summarized (mean, standard deviation, coefficient of variation, median, and minimum and maximum).

Inter-participant variability is evaluated.

An exploratory and descriptive analysis of the potential relationship between PK and other endpoints (e.g., safety, efficacy, PD etc.) may be conducted.

PK data may be pooled across studies for population PK analyses. These analyses may be reported separately from the CSR. Additional PK analyses are conducted as appropriate.

### Immunogenicity Analyses

The immunogenicity analysis population consists of all participants with at least one post-dose anti-drug antibody (ADA) assessment.

The number and proportion of ADA-positive participants and ADA-negative participants at baseline (baseline prevalence) and after drug administration (postbaseline incidence) are summarized. When determining postbaseline incidence, participants are considered to be ADA positive if they are ADA negative or have missing data at baseline but develop an ADA response following study drug exposure (treatment-induced ADA response), or if they are ADA positive at baseline and the titer of one or more postbaseline samples is at least 0.60-titer unit greater than the titer of the baseline sample (treatment-enhanced ADA response). Participants are considered to be ADA negative if they are ADA negative or have missing data at baseline and all postbaseline samples are negative, or if they are ADA positive at baseline but do not have any postbaseline samples with a titer that is at least 0.60-titer unit greater than the titer of the baseline sample (treatment unaffected).

The relationship between ADA status and safety, efficacy, PK or biomarker endpoints may be analyzed and reported via descriptive statistics.

### H. SLE Background Medication Tapering and Discontinuation

The methods described herein evaluate whether mosunetuzumab administration induces deep-tissue and peripheral B-cell depletion and whether deep-tissue and peripheral B-cell depletion confers a clinical response that allows for the safe discontinuation of background SLE medications, with the primary endpoint of achieving sustained drug-free remission. Standardized discontinuation guidance for background SLE medications is provided to ensure participant safety during therapy withdrawal.

### Hydroxychloroquine

Participants continue their hydroxychloroquine dose throughout the study.

### Oral Corticosteroids

Oral corticosteroid tapering begins early, e.g., at Visit (Day) 1 (baseline). Duration of steroid taper is dependent on the starting dose of OCS. Guidance for tapering is provided in Table 9 below.

TABLE 9
Corticosteroid Tapering Schedule

| Study Visit | Dose of Oral Prednisone or Equivalent (mg per day) | | | | |
|---|---|---|---|---|---|
| Day 1 | 10 | 20 | 30 | 40 | 50 |
| Week 2 | 7.5 | 15 | 25 | 30 | 40 |
| Week 4 | 7.5 | 10 | 20 | 20 | 30 |
| Week 8 | 5 | 7.5 | 15 | 15 | 20 |
| Week 12 | 5 | 5 | 10 | 10 | 10 |
| Week 16 | 0 | 5 | 7.5 | 7.5 | 7.5 |
| Week 20 | 0 | 0 | 5 | 5 | 5 |
| Week 24 | 0 | 0 | 0 | 0 | 0 |

### Calcineurin Inhibitors (CNI)

Due to the potential risk of rebound proteinuria following CNI discontinuation, CNIs are also tapered early in the study. Tapering should be completed prior to the last dose of mosunetuzumab.

### Other Background Immunosuppressant Therapies

In order for the discontinuation of other immunosuppressant therapies (e.g. MMF, azathioprine (AZA)) to be considered, it is recommended that participants meet certain eligibility criteria. The suggested criteria for discontinuing background immunosuppressants are found in Table 10.

TABLE 10
Suggested Criteria for Discontinuation of Other Immunosuppressant Therapies

| Cohort 1 | Cohort 2 |
|---|---|
| cSLEDAI = 0* | cSLEDAI = 0 |
| Documented peripheral B-cell depletion by | Documented peripheral B-cell depletion by |
| B, T, and NK cell subset flow cytometry at any | B, T, and NK cell subset flow cytometry at any |
| time point after start of mosunetuzumab | time point after start of mosunetuzumab |
| treatment* | treatment |
| NIH activity index of 0-1 on repeat kidney | Improvement in symptoms not included in |
| biopsy | SLEDAI-2K |
| Persistent proteinuria can be explained by | |
| findings not associated with active LN disease | |
| eGFR is stable or improving | |

Additional criteria that can be considered for when to discontinue background immunosuppressant therapy may include, but are not limited to, the stability of the kidney biopsy chronicity index in patients with lupus nephritis, normalization of complement levels, and improvement in SLE-specific autoantibody levels.

The Background Immunosuppressant Discontinuation Eligibility Point or BDEP is the time point at which the investigator deems it is appropriate to discontinue standard background immunosuppressant therapy based on the participant's disease activity. Once a participant reaches the BDEP, other immunosuppressant therapies, specifically MMF and azathioprine (AZA), should be discontinued without tapering. The decision to taper and discontinue immunosuppression is ultimately at the discretion of the investigator based on what they deem is safe and clinically appropriate for each participant.

### Example 2. A Phase Ib Study to Evaluate the Effects of Mosunetuzumab in Patients with Systemic Lupus Erythematosus

### A. Description of Study

This was a Phase Ib multicenter, open-label, dose-escalation study to evaluate the safety, tolerability, pharmacokinetics, and pharmacodynamics of mosunetuzumab in participants with SLE (NCT05155345). The study enrolled up to 50 participants with SLE at approximately 15 investigative sites globally. Approximately 13-25 participants were enrolled during the dose-finding and dose-escalation periods. Participants withdrawn during the dose-limiting toxicity (DLT) assessment period for any reasons other than a DLT were replaced. Additional participants were able to be added during the dose expansion period to replace participants who discontinued prematurely, expand an existing dose cohort, fill a cohort at a modified dose, or assess a particular subgroup of interest (e.g., participants receiving a specific permitted SLE background therapy). The characteristics of the patient population are described in Table 11. At time of clinical cutoff date, 15 adult participants diagnosed with active autoantibody-positive SLE demonstrated by a Systemic Lupus Erythematosus Disease Activity Index 2000 (SLEDAI-2K) total score ≥4 at screening were enrolled.

TABLE 11
Phase Ib Baseline Characteristics of Patient Population

| | Mean (Standard | | | Interquartile | Reference | |
|---|---|---|---|---|---|---|
| | Deviation) | | Median | Range | Range | |
| Gender (female), | 14 | (93.3%) | | | N/A | |
| n (%) | | | | | | |
| Age | 47 | (15.6) | 51 | (35.0, 57.5) | N/A | |
| SLEDAI-2K score | 7.5 | (3.0) | 6.0 | (6.0, 10.0) | N/A | |
| at screening | | | | | | |
| Complement | 110.1 | (36.3) | 105.0 | (85.5, 142.5) | 90-180 | mg/dL |
| protein C3 | | | | | | |
| Complement | 17.7 | (13.0) | 16.1 | (9.1, 21.4) | 10-40 | mg/dL |
| protein C4 | | | | | | |
| Anti-double | 43.8 | (60.7) | 12.3 | (12.3, 41.2) | <75 | IU/mL |
| stranded DNA | | | | | | |
| autoantibodies, | | | | | | |
| International | | | | | | |
| units (IU)/mL | | | | | | |

| | | | | | |
|---|---|---|---|---|---|
| B cell (high | 244.8 | (159.1) | 192.5 | (147.4, 299.4) | Not available |
| sensitivity) | | | | | Lower limit of |
| | | | | | quantification = |
| | | | | | 0.441 cells/μL |

*3 patients in the cohort receiving a first dose of 5 mg mosunetuzumab and a second dose of 60 mg mosunetuzumab were on stable MMF background therapy >4 weeks before screening

The study consisted of the following study periods: screening, dose finding, dose escalation, dose-expansion, and safety follow-up (SFU). Participants in the non-fractionated/dose-finding cohorts received a non-fractionated dose of mosunetuzumab on Day 1, while participants in the fractionated/dose-escalation cohorts received a fractionated dose administered on Day 1 and Day 8. The duration of SFU was a minimum of 12 months or longer depending on a patient participant's time to B cell repletion. In the dose-expansion period, participants may be added to expand a cohort, explore an alternate dose, and/or assess participant subgroups.

### Dose Finding

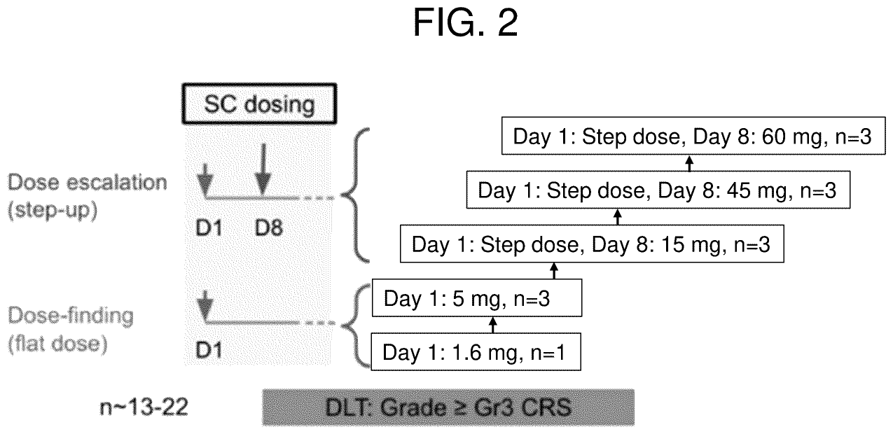

The non-fractionated/dose-finding cohorts of the study were comprised of 1 participant allocated to receive a single dose of 1.6-mg mosunetuzumab followed by participants who received a single 5-mg dose of mosunetuzumab, respectively. The 5-mg cohort was conducted using a 3+3 design consisting of at least 3 participants for up to a total of 6 participants. In the event that DLTs were observed in the first 2 participants in the 5-mg cohort, then only 2 participants were enrolled in the 5-mg cohort (FIG. 2).

In order to assess for any severe and unexpected acute drug or injection-related toxicities, dosing of participants was staggered by at least 72 hours in the dose finding and dose escalation phases, and 24 hours apart for dose-expansion participants. All participants were hospitalized for 72 hours.

The step dose that was utilized as the Day 1 dose during the dose-escalation period was informed by the totality of the clinical data obtained during the dose-finding period.

A further breakdown of the 15 patients enrolled in the study described in Table 11 above by dose received and with additional baseline characteristics is shown below in Table 12.

TABLE 12
Baseline Characteristics of Mosunetuzumab Dose Cohorts

| | | | 5 mg + | 5 mg + | 5 mg + | |
|---|---|---|---|---|---|---|
| | 1.6 mg | 5 mg | 15 mg | 45 mg | 60 mg | All |
| N | 1 | 3 | 4 | 4 | 3 | 15 |
| Age (yrs) | | | | | | |
| mean (SD) | 68.0 (NE) | 56.0 (10.5) | 49.0 (16.5) | 50.5 (8.1) | 25.3 (3.2) | 47.3 (15.6) |
| Female n (%) | 1 (100%) | 3 (100%) | 4 (100%) | 3 (75.0%) | 3 (100%) | 14 (93.3%) |
| Race n (%) | | | | | | |
| Black/African American | 1 (100%) | 0 (0.0%) | 1 (25.0%) | 0 (0.0%) | 0 (0.0%) | 2 (13.3%) |
| White | 0 (0.0%) | 3 (100%) | 3 (75%) | 4 (100%) | 3 (100%) | 13 (86.7%) |
| Ethnicity^{a} n (%) | | | | | | |
| Not Hispanic or Latino | 0 (0.0%) | 3 (100%) | 4 (100%) | 4 (100%) | 3 (100%) | 14 (93.3%) |
| Geographical region n (%) | | | | | | |
| Europe | 0 (0.0%) | 0 (0.0%) | 1 (25.0%) | 2 (50.0%) | 3 (100%) | 6 (40.0%) |
| North America | 1 (100%) | 3 ( 100%) | 3 (75.0%) | 2 (50.0%) | 0 (0.0%) | 9 (60.0%) |
| Total SLEDAI-2K score | | | | | | |
| mean (SD) | 6.00 (NE) | 5.33 (2.31) | 7.50 (3.00) | 8.00 (3.65) | 9.33 (3.06) | 7.47 (2.97) |
| Anti-dsDNA antibody status^{b} | | | | | | |
| Positive | 0 (0.0%) | 0 (0.0%) | 1 (33.3%) | 1 (25.0%) | 1 (33.3%) | 3 (21.4%) |
| Negative | 1 (100%) | 3 (100%) | 2 (66.7%) | 2 (50.0%) | 1 (33.3%) | 9 (64.3%) |
| Borderline | 0 (0.0%) | 0 (0.0%) | 0 (0.0%) | 1 (25.0%) | 1 (33.3%) | 2 (14.3%) |
| Complement C3 level^{c} | | | | | | |
| mean (SD) | 1.51 (NE) | 1.33 (0.32) | 1.15 (0.49) | 0.87 (0.33) | 0.98 (0.09) | 1.10 (0.36) |
| Complement C4 level^{c} | | | | | | |
| mean (SD) | 0.16 (NE) | 0.21 (0.08) | 0.21 (0.24) | 0.15 (0.07) | 0.15 0.06) | 0.18 (0.13) |

dsDNA: double-stranded DNA; NE: not estimable; SD: standard deviation; SLEDAI-2K: Systemic Lupus Erythematosus Disease Activity Index 2000.
^{a}The ethnicity of one participant was unknown.
^{b}The following cut-offs were used for anti-dsDNA antibody categories: negative: <30 IU/mL; borderline: 30-75 IU/mL; positive: >75 IU/mL.
^{c}Central laboratory results.

### Dose Escalation

Fractionated dosing on Day 1 and Day 8 was planned for dose escalation. Participants received the step-dose (determined by dose-finding period) on Day 1, followed by doses of 15 mg, 45 mg, or 60 mg on Day 8 for the respective fractionated/dose-escalation cohorts. Dose escalation was conducted using a 3+3 design consisting of at least 3 participants for up to a total of 6 participants for each fractionated cohort. In each fractionated cohort, the Day 1 dose for each participant must have been separated from the previous participant's Day 1 dose by an interval of at least 72 hours (FIG. 2).

In this study, an interval of at least 7 days between the last participant in the non-fractionated dose-finding cohort and the first participant of the first, fractionated dose-escalation cohort was instituted to allow completion of the DLT assessment period for the non-fractionated dose-finding cohort and review of data by the Internal Monitoring Committee (IMC). Similarly, subsequent dose escalation cohorts were also separated by an interval of at least 7 days from the preceding cohort's last participant's last dose to allow completion of the DLT assessment period and review of data by the IMC.

As in the non-fractionated dose-finding cohorts, dosing of participants was staggered by at least 72 hours, and all participants were hospitalized for 72 hours following the Day 1 and Day 8 doses. Hospitalization requirements were reassessed at regular intervals by the IMC as more safety, PK, and PD information from participants receiving fractionated dosing became available throughout dose escalation.

Following completion of the DLT assessment period (7-14 days), all participants were closely monitored for adverse events throughout the study and during the SFU. Adverse events were graded according to NCI CTCAE v5.0, with CRS graded according to the ASTCT 2019 CRS Consensus Grading Criteria.

The IMC reviewed safety data and made recommendations regarding dose-escalation and overall study conduct to ensure participant safety while receiving study treatment.

### Dose Escalation Rules

All dose-escalation decisions were made based on recommendations of the IMC. Safety information, including relevant adverse events and clinical laboratory data, were reviewed prior to each dose escalation decision. On the basis of a review of real-time clinical data, dose escalation was able to be halted or modified by the Sponsor as deemed appropriate.

In the dose-escalation period, the decision to proceed to the next higher dose was conducted in accordance with the following rules based on the 3+3 dose escalation design:
A minimum of 3 participants were enrolled in the cohort unless the first 2 enrolled participants experienced a DLT, in which case enrollment into the cohort was terminated.
If none of the first 3 DLT-evaluable participants experienced a DLT, enrollment of the next dose-escalation cohort may have proceeded upon completion of the DLT assessment period and IMC confirmation.
If 1 of the first 3 DLT-evaluable participants experienced a DLT, the cohort was expanded to at least 6 participants. All participants were evaluated for DLTs before any dose-escalation decision was made.
   If DLTs were observed in 17% of participants in a given cohort (e.g., DLTs observed in 1 of 6 DLT-evaluable participants), enrollment of the next dose-escalation cohort may have proceeded.
   If DLTs were observed in 17% of participants in a given cohort, further enrollment at that dose level and dose-escalation was halted.

### B. Mosunetuzumab Demonstrated an Acceptable Safety Profile

Clinical data cutoff of Jun. 6, 2024. Analysis is still ongoing.

No immune effector cell-associated neurotoxicity syndrome (ICANS) occurred in any of the patients.

Patients were evaluated for whether they experienced a cytokine release syndrome (CRS) event in response to dosing with mosunetuzumab. CRS events were graded according to the ASTCT criteria. In total out of the 15 patients in the study, two patients experienced at least one CRS event, with a highest Grade of Grade 1, and two patients experienced at least one CRS event, with a highest Grade of Grade 2. Three Grade 1 CRS events were associated with Day 1 injection, one Grade 2 CRS event was associated with Day 1 injection, and 1 Day 2 CRS event was associated with Day 8 injection (e.g., of mosunetuzumab). No Grade 3 or higher (high grade) CRS events occurred.

Adverse events (AEs) were graded according to NCI CTCAE v5.0. 11 patients reported a Grade 1 injection site reaction, and one patient reported a Grade 2 injection site reaction. Two patients reported a Grade 1 infection/infestation, and nine patients reported a Grade 2 infection/infestation. Most common infections include COVID-19 (3 patients; 20%), urinary tract infections (3 patients; 20%), upper respiratory tract Infections (2 patients; 13.3%). Four patients reported a Grade 1 headache, and one patient reported a Grade 2 headache. Three patients reported a Grade 1 pyrexia. Two patients reported a Grade 1 rash, and one patient reported a Grade 2 rash (rash terms included “rash” and “dermatitis contact”). One patient reported a Grade 2 neutropenia/neutrophil count decrease, and one patient reported a Grade 3 neutropenia/neutrophil count decrease. Two patients reported a Grade 1 diarrhea. One patient reported a Grade 1 dyspepsia, and one patient reported a Grade 2 dyspepsia. Two patients reported a Grade 2 foot fracture. Two patients reported a Grade 2 pain. One patient in the 5+45 mg cohort died from interstitial pneumonia 37 days after the end of the DLT assessment period. The death was deemed unrelated to mosunetuzumab by the investigator and the sponsor. Only 2 patients experienced a Grade 3+ AE (1 non-serious Grade 3 neutropenia) and 1 serious AE occurred (Grade 5 interstitial lung disease).

### Non-Fractionated Doses

The patient who received 1.6 mg mosunetuzumab did not experience CRS. Of the five patients who received 5 mg mosunetuzumab, one patient experienced a Grade 1 CRS (14 hours after Day 1 dosing), and one patient experienced a Grade 2 CRS (12 hours after Day 1 dosing with a fever of 39.1 degrees Centigrade and hypotension of 105/50 mmHg).

### Fractionated Doses

Of the three patients who received 5 mg mosunetuzumab and then 15 mg mosunetuzumab, none experienced CRS. Of the three patients who received 5 mg mosunetuzumab and then 45 mg mosunetuzumab, one patient experienced both a Grade 1 CRS (14.5 hours after Day 1 dosing) and a Grade 2 CRS (48 hours after Day 8 dosing with hypotension of 86/56 mmHg, hypoxia oxygen saturation of 89%, and temperature of 37.6 C). Of the three patients who received 5 mg mosunetuzumab and then 60 mg of mosunetuzumab, only one patient experienced a Grade 1 CRS (24 hours after Day 1 dosing).

Overall, safety findings were acceptable with only 4 out of 15 total patients experiencing Grade 1 or 2 CRS. Most incidences of CRS were after Day 1 dosing, only one Grade 2 CRS occurred after Day 8 dosing. High IL-6 levels appeared to be associated with CRS for most but not all patients.

### C. Mosunetuzumab Demonstrated Pharmacodynamic Efficacy for SLE

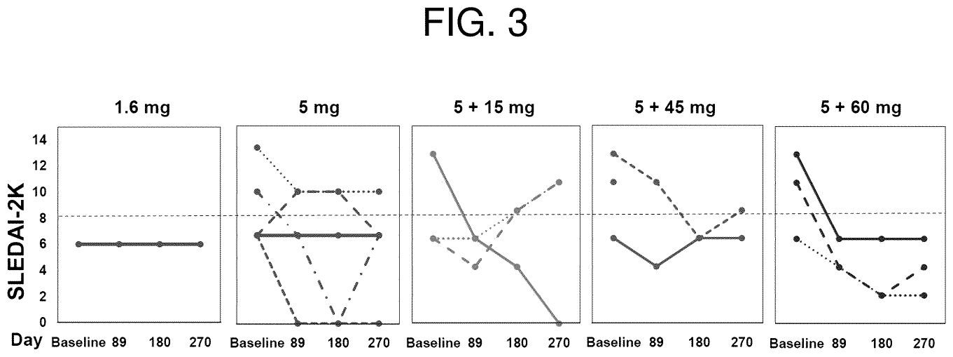

The effects of mosunetuzumab on symptoms of lupus were evaluated. SLEDAI-2K scores decreased, although not to 0. Specifically, out of six patients with baseline SLEDAI-2K scores of at least eight, four patients had a decrease in SLEDAI-2K scores of at least four points (FIG. 3).

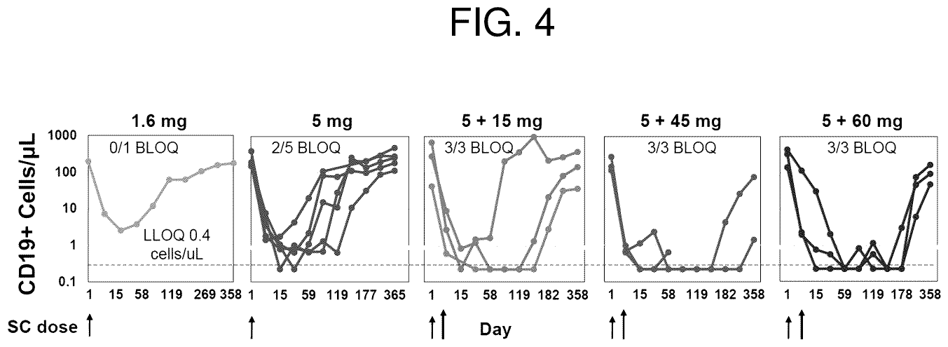

The concentration of peripheral B cells after SC doses of mosunetuzumab was also evaluated. The concentration of B cells was depleted to below the limit of quantification of 0.4 cells/μL for at least one timepoint for all nine patients who received fractionated doses. The greatest depletion of peripheral B cells occurred at doses of 5 mg mosunetuzumab followed by 45 mg mosunetuzumab or with doses of 5 mg mosunetuzumab followed by 60 mg mosunetuzumab. Nine out of nine patients who received fractionated doses achieved B cell levels below the limit of quantification for at least one timepoint. Seven out of the nine patients who received fractionated doses achieved B cell levels below the limit of quantification for at least 14 days. All patients who received the three highest doses (5+15 mg, 5+45 mg, and 5+60 mg) achieved B cell depletion to below 0.4 cells/μL. The presence of mycophenolate mofetil (MMF) did not block depletion as all patients with MMF showed B cell depletion below the limit of quantification BLOQ (FIG. 4).

The depth (i.e., extent) and duration of B cell depletion appeared to increase with exposure to mosunetuzumab. There was a profound depletion of CD19+ B cells in almost all patients after two weeks (CD19+ B cell ≤5 cells/μL), regardless of exposure quartile. The duration during which a population exhibited profound depletion in 100% of patients depended upon the amount of exposure to mosunetuzumab. For the first, second, and third quartiles of exposure to mosunetuzumab, profound depletion in 100% of patients lasted up to the third, sixth, and ninth months, respectively. After just two weeks, 0%, 50%, 50%, and 66.7% of patients in the first, second, third, and fourth quartiles of mosunetuzumab exposure, respectively, showed near-complete depletion of B cells (CD19+ B cells s 0.441 cells/μL). The time to profound and time to near-complete depletion of B cells was shorter for the fourth quartile in patients in terms of mosunetuzumab exposure. Patients at quartiles 1 and 2 of exposure began to experience complete repletion of B cells (CD19+ B cells ≥72 cells/μL) after 3 months, whereas no patient at higher exposure quartiles showed a complete repletion by month 9. Overall, there was an apparent relationship between mosunetuzumab exposure and peripheral cell depletion and repletion kinetics, with higher exposures associated with shorter times to depletion, longer durations of depletion, and longer times to repletion, although the number of patients in the study was limited. Fourth quartile of exposure showed deeper systemic B cell depletion and slower B cell repletion to normality as compared with other exposure quartiles (e.g., wherein patients could maintain B cell depletion at the ninth month).

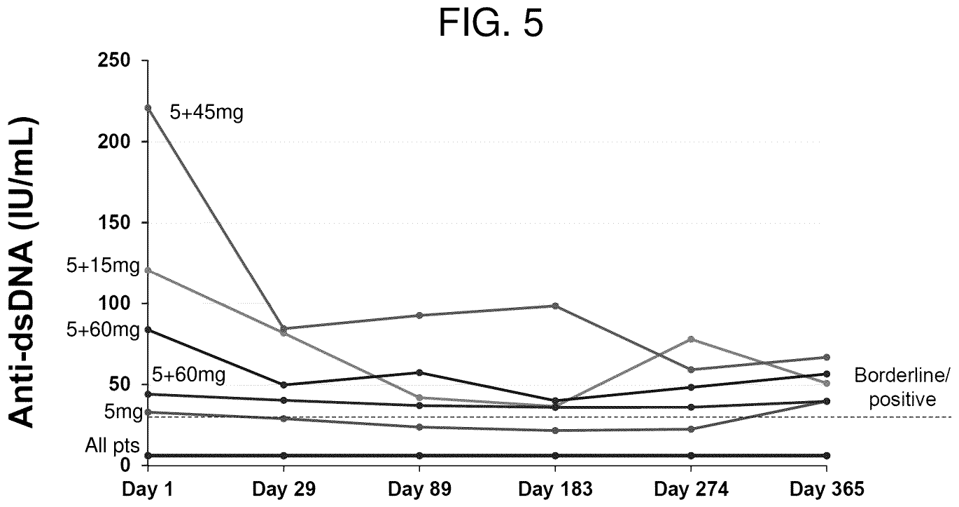

Additionally, lupus-related biomarkers responded to treatment with mosunetuzumab. The level of anti-dsDNA antibodies decreased after treatment with doses of mosunetuzumab. Anti-dsDNA antibody levels decreased in four out of five patients with borderline/positive levels at baseline (FIG. 5). All five patients with positive anti-dsDNA antibody levels at baseline exhibited a diminution in positivity of their anti-dsDNA antibody levels during the study.

Lupus patients often have low levels of complement proteins C3 and C4 in the periphery because it is thought that these proteins trigger tissue cellular toxicity. Because the patient population in this study had milder disease, most patients were in the normal range. Nonetheless, one out of four patients went from low to normal levels of C3, and four out of five patients increased from low to normal levels of C4.

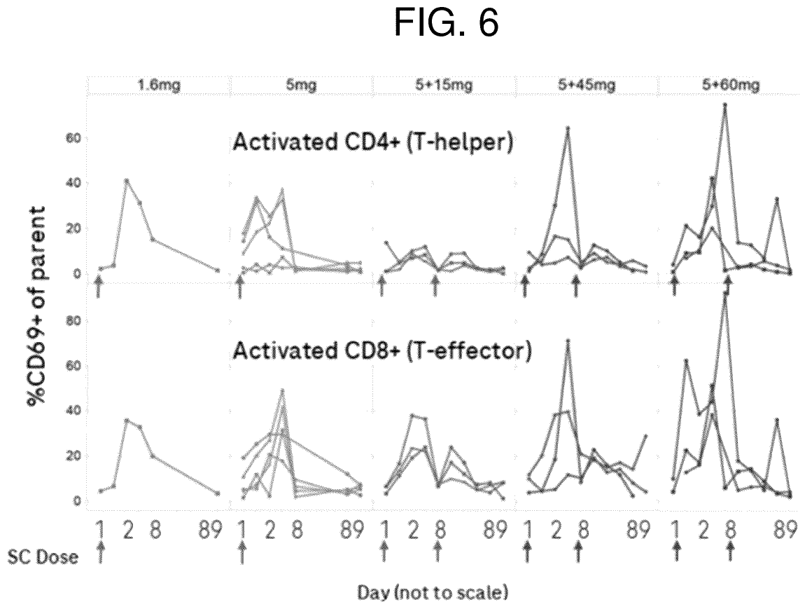

A PD biomarker of mosunetuzumab is the activation of T cells expressing CD69. Patients showed increases in the percent of T cells that were activated after mosunetuzumab administration in both CD4+T-helper and CD8+T-effector subsets as early as one day after dosing (FIG. 6). Increases were also observed in T cells expressing the cytotoxic Granzyme B, which is secreted during B cell killing, and in the proliferation marker Ki67. T-cell activation occurred in all doses, including the 5+60 mg dose that was on background mycophenolate mofetil. This provided more evidence that mosunetuzumab functioned in lupus patients consistent with its mechanism of action in oncology.

This study demonstrated, for the first time, pharmacodynamic changes in biomarkers and proof of activity of mosunetuzumab in lupus patients showing B cell depletion and T cell activation.

### D. Mosunetuzumab Exhibited PK Dynamics in SLE Patients Similar to Those Demonstrated for FL Patients

The PK exhibited for mosunetuzumab in SLE (e.g., for subcutaneously administered mosunetuzumab) appeared similar to the PK observed in follicular lymphoma (FL) patients treated with subcutaneously administered mosunetuzumab. In addition, an apparent dose-dependent increase in PK was observed.

### E. Quantitative Systems Pharmacology (QSP) Modeling Guided Dose Selection

QSP modeling was used to guide dose selection for SLE Phase Ib study and leveraged to guide Phase 2 dosing.

The PK and PD of mosunetuzumab were simulated based on mosunetuzumab data in FL, diffuse large B cell lymphoma, and Phase 1b mosunetuzumab in SLE study and cynomolgus macaque data. PK simulations were based on mean peripheral B cell depletion, not tissue depletion. QSP modeling suggested that dose level (DLs) ≥15 mg Q1 M as maintenance (with 45 mg 1st cycle Day 15 dose) may result in ≥90% patients with sustained B cell depletion.

Simulations suggested maintenance doses of 30 mg mosunetuzumab or 15 mg mosunetuzumab administered every two months achieved B cell depletion in >90% patients after one month and in 80-100% of patients in the maintenance phase after initial step doses of 1.5 mg mosunetuzumab, 5 mg mosunetuzumab, and 45 mg mosunetuzumab. Similarly, simulations suggested maintenance doses of 15 mg mosunetuzumab or 5 mg mosunetuzumab administered every month achieved B cell depletion in ≥90% patients after one month and in 80-100% of patients in the maintenance phase after initial step doses of 1.5 mg mosunetuzumab, 5 mg mosunetuzumab, and 45 mg mosunetuzumab.

QSP modeling suggested that one dose every month (Q1 M) did not significantly differ as compared to one dose every three weeks (Q3W) at DLs ≥15 mg as maintenance doses with respect to peripheral B cell depletion.

With maintenance dosing every two months (Q2M) or every three months (Q3M), QSP modeling suggested a decrease in the percent of patients with B cell depletion at maintenance dose levels under consideration (15-60 mg).

Exposure-response (ER) analysis supports the use of 45 mg as maintenance dose, as there is an apparent ER (B cell depletion and repletion) relationship with shorter time to depletion, longer duration of depletion, and longer time to repletion, generally seen with increasing exposures, though sample size is limited. The groups receiving mosunetuzumab doses of 5 mg and then 45 mg or of 5 mg and then 60 mg showed similar PK profiles and exposures.

QSP models were also used to simulate IL-6 levels after mosunetuzumab doses. There was a lower simulated IL-6 peak after a first dose of 1.6 mg mosunetuzumab compared to 5 mg of mosunetuzumab. There was a lower simulated IL-6 peak after administration of a second dose of mosunetuzumab at a dose of 5 mg, 15 mg, 45 mg, or 60 mg, compared to the simulated IL-6 peak after a first dose of 5 mg of mosunetuzumab or a first dose of 1.6 mg of mosunetuzumab. Higher second doses of mosunetuzumab correlated with higher simulated peaks of IL-6 levels. Overall, the simulated data suggest that a lower first dose (e.g., 1.6 mg as compared to 5 mg), will trigger lower IL-6 levels after administration of the first dose on Day 1. A similar concept applies for the second dose, i.e., administration of a lower second dose (e.g., 5 mg dose as compared to higher doses of 15 mg, 45 mg, or 60 mg) also results in lower IL-6 levels. In addition, because the first dose is 1.6 mg and target dose is 45 mg, the simulated data suggested adding a second dose between the 1.6 mg first dose and the 45 mg target dose to avoid large jumps in dose escalation. The totality of the simulation data described above suggests administration of two escalation steps, i.e., 1.6 mg and 5 mg on Days 1 and 8, respectively, before administration of the target dose of 45 mg on Day 15.

The impact of an additional 45 mg mosunetuzumab dose on Day 15 PK was also simulated. A dosing regimen of 5 mg mosunetuzumab on Day 1, 45 mg mosunetuzumab on Day 8, and 45 mg mosunetuzumab on Day 15 enabled higher early exposures compared to a dosing regimen of 5 mg mosunetuzumab on Day 1 and 45 mg mosunetuzumab on Day 45. There was a minimal impact of the 45 mg mosunetuzumab dose on Day 15 on IL-6 levels after Day 15, and peripheral B cell depletion was comparable between the dosing regimens.

QSP modeling data for PK and PD was overlaid with observed data from patients with SLE. The simulated data correlated with the empirical data. QSP models that included the tissue B cell compartment captured a second IL-6 peak, which was observed in the empirical data. Based on QSP modeling, B cell depletion profile at population level and individual level suggested 95%-100% of patients maintain B cell depletion with a dosing schedule of 1.5/5/45 mg, followed by monthly dosing of 30 mg or higher.

### F. Conclusion and Considerations

### Number of Step-Up Doses

Three-dose step up (in incremental doses) may help to fully mitigate CRS risk and need for hospitalization. Two-dose step up from a single dose Phase Ib study had an acceptable CRS risk profile, though the sample size was limited. There is potential for heightened sensitivity to on-target effects in SLE vs NHL due to higher baseline circulating B cell levels.

### First Cycle Full Dose and Maintenance Dose

A single fractionated dose 5/45 mg DL showed sustained peripheral B cell depletion over several months in the Phase Ib study although there was a limited sample size. All fractionated cohorts (5/15, 5/45, 5/60 mg) showed B cell levels below the limit of quantification for at least one time point for B cell depletion. It was difficult to distinguish between DLs due to limited sample size. QSP modeling suggested that DLs ≥15 mg every month as maintenance (with 45 mg 1st cycle Day 15 dose) may result in ≥90% patients with sustained B cell depletion. Peripheral B cell depletion may differ as compared to tissue-level depletion which may be required for clinical remission.

### Dosing Frequency

QSP modeling suggested every month dosing may not significantly differ as compared to dosing every three weeks at DLs ≥15 mg as maintenance with respect to peripheral B cell depletion. With every two month or every three month dosing frequency, QSP modeling suggested decreases in the percent of patients with B cell depletion at maintenance dose levels under consideration (30 or 45 mg). Single fractionated dosing of mosunetuzumab (5 mg and then 15 mg, 5 mg and then 45 mg, or 5 mg and then 60 mg) in this Phase Ib study showed decreases in SLEDAI-2K score by ≥4 (in 4/6 patients with baseline ≥8), however, not reaching 0 for most, suggesting that higher exposures or more frequent dosing might be required.

### Dosing Duration

The following were considered for dosing duration:
Fixed dosing against response-based dosing preferable to support Phase II objectives
6 months dosing comparable to match CAR-T kinetics
Mosunetuzumab is dosed much longer (up to—17 cycles in FL patients with partial response (PR) or stable disease (SD) by the end of cycle 8 (~6 months); however, risk tolerance is different for FL vs SLE patients.

### Overall

Preliminary data from this Phase Ib study indicated that mosunetuzumab exhibits an acceptable safety profile in patients with SLE and that its PK profile in the SLE population is consistent with that observed in the relapsed or refractory Non-Hodgkin lymphoma population. Deep B-cell-depletion was observed in the higher-dose cohorts. While this study was designed to evaluate the safety and PK profile of mosunetuzumab with a limited dosing regimen, a positive impact on clinical activity in participants who had higher SLEDAI-2K scores and anti-dsDNA antibody levels at baseline was observed.

### EMBODIMENTS

Some embodiments of the technology described herein can be defined according to any of the following numbered embodiments:
1. A method of treating an individual having systemic lupus erythematosus (SLE), comprising subcutaneously administering to the individual mosunetuzumab according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
2. Use of mosunetuzumab for treating systemic lupus erythematosus (SLE) in an individual in need thereof, wherein mosunetuzumab is to be administered subcutaneously to the individual according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
3. Use of mosunetuzumab in the manufacture of a medicament for use in a method of treating systemic lupus erythematosus (SLE) in an individual in need thereof, wherein mosunetuzumab is formulated for subcutaneous administration to the individual according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
4. Mosunetuzumab for use in treating systemic lupus erythematosus (SLE) in an individual in need thereof, wherein mosunetuzumab is to be administered subcutaneously to the individual according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
5. The method, use, or mosunetuzumab for use of any one of embodiments 1-4, wherein the first dosing cycle is a 21-day dosing cycle.
6. The method, use, or mosunetuzumab for use of any one of embodiments 1-5, wherein the C1D1, C1D2, and C1D3 are administered or to be administered on or about Days 1, 8, and 15, respectively, of the first dosing cycle.
7. The method, use, or mosunetuzumab for use of any one of embodiments 1-6, wherein the second dosing cycle is a 21-day dosing cycle.
8. The method, use, or mosunetuzumab for use of any one of embodiments 1-7, wherein the C2D1 is administered or to be administered on or about Day 1 of the second dosing cycle.
9. The method, use, or mosunetuzumab for use of any one of embodiments 1-8, wherein the method further comprises one or more additional dosing cycles.
10. The method, use, or mosunetuzumab for use of embodiment 9, wherein the method comprises one to seven additional dosing cycles.
11. The method, use, or mosunetuzumab for use of embodiment 9 or 10, wherein the method comprises seven additional dosing cycles.
12. The method, use, or mosunetuzumab for use of any one of embodiments 9-11, wherein each additional dosing cycle is a 21-day dosing cycle.
13. The method, use, or mosunetuzumab for use of any one of embodiments 9-12, wherein one or more of the additional dosing cycles comprises an additional single dose of mosunetuzumab.
14. The method, use, or mosunetuzumab for use of embodiment 13, wherein the additional single dose of mosunetuzumab is administered or to be administered on Day 1 of the respective additional dosing cycle.
15. The method, use, or mosunetuzumab for use of embodiment 13 or 14, wherein the additional single dose of mosunetuzumab is about 45 mg.
16. A method of treating an individual having SLE, comprising subcutaneously administering to the individual mosunetuzumab according to a dosing regimen comprising nine 21-day dosing cycles, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are administered on Days 1, 8, and 15, respectively, of the first dosing cycle; and
(b) the second to ninth dosing cycles each comprises a single dose (C2D1-C9D1) of mosunetuzumab administered on Day 1 of each respective dosing cycle, wherein each of the C2D1-C9D1 is about 45 mg.
17. Use of mosunetuzumab for treating systemic lupus erythematosus (SLE) in an individual in need thereof, wherein mosunetuzumab is to be administered subcutaneously to the individual according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
18. Use of mosunetuzumab in the manufacture of a medicament for use in a method of treating systemic lupus erythematosus (SLE) in an individual in need thereof, wherein mosunetuzumab is formulated for subcutaneous administration to the individual according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
19. Mosunetuzumab for use in treating systemic lupus erythematosus (SLE) in an individual in need thereof, wherein mosunetuzumab is to be administered subcutaneously to the individual according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
20. The method, use, or mosunetuzumab for use of any one of embodiments 1-19, wherein mosunetuzumab is administered or to be administered as a monotherapy.
21. The method, use, or mosunetuzumab for use of any one of embodiments 1-20, wherein prior to administration of any dose of mosunetuzumab, the individual has been previously administered an oral corticosteroid, an antimalarial agent, or an immunosuppressant.
22. The method, use, or mosunetuzumab for use of any one of embodiments 1-21, wherein the individual has been administered an antimalarial agent at a stable dose for at least 4 weeks prior to being administered mosunetuzumab.
23. The method, use, or mosunetuzumab for use of embodiment 23, wherein the antimalarial agent is hydroxychloroquine, chloroquine, or quinacrine.
24. The method, use, or mosunetuzumab for use of any one of embodiments 1-21, wherein the individual has been administered an immunosuppressant at a stable dose for at least 4 weeks prior to being administered mosunetuzumab.
25. The method, use, or mosunetuzumab for use of embodiment 24, wherein the immunosuppressant is azathioprine, mycophenolate mofetil, mycophenolic acid, or methotrexate.
26. The method, use, or mosunetuzumab for use of any one of embodiments 1-25, wherein the individual does not have a lupus-associated neuropsychiatric disease.
27. The method, use, or mosunetuzumab for use of embodiment 26, wherein the lupus-associated neuropsychiatric disease is meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, or mononeuritis multiplex.
28. The method, use, or mosunetuzumab for use of any one of embodiments 1-27, wherein the individual does not have active overlap syndrome with mixed connective tissue disease or systemic sclerosis within a year prior to being administered mosunetuzumab.
29. The method, use, or mosunetuzumab for use of any one of embodiments 1-28, wherein the individual does not have catastrophic or severe antiphospholipid syndrome within a year prior to being administered mosunetuzumab, unless the severe antiphospholipid syndrome has been adequately controlled by administering anticoagulant therapy to the individual at least 2 months prior to being administered mosunetuzumab.
30. The method, use, or mosunetuzumab for use of any one of embodiments 1-29, wherein the individual has not been administered:
(a) at least 12 months prior to being administered mosunetuzumab:
   (i) an anti-CD19 antibody therapy, or
   (ii) an anti-CD20 monoclonal antibody therapy;
(b) at least 30 days prior to being administered mosunetuzumab:
   (i) kinase inhibitors of Janus Kinase (JAK) kinase, Bruton tyrosine kinase, or tyrosine kinase 2, or
   (ii) a calcineurin inhibitor; or
(c) at least 2 months prior to being administered mosunetuzumab:
   (i) cyclophosphamide, or
   (ii) a biologic therapy.
31. The method, use, or mosunetuzumab for use of embodiment 30, wherein:
(a) the anti-CD19 therapy is blinatumomab;
(b) the anti-CD20 therapy is obinutuzumab, rituximab, ocrelizumab, or ofatumumab;
(c) the kinase inhibitors are baricitinib, tofacitinib, upadacitinib, filgotinib, ibrutinib, or fenebrutinib;
(d) the calcineurin inhibitor is tacrolimus, ciclosporin, or voclosporin; or
(e) the biologic therapy is belimumab, ustekinumab, anifrolumab, secukinumab, or atacicept.
32. The method, use, or mosunetuzumab for use of any one of embodiments 1-31, wherein the individual does not have significant lupus-associated renal disease or significant renal impairment.
33. The method, use, or mosunetuzumab for use of any one of embodiments 1-32, wherein the individual does not have any of the following laboratory parameters:
(a) total bilirubin >1.5×upper limit of normal (ULN);
(b) absolute neutrophil count (ANC)<1.5×10⁹/L (<1500/μL);
(c) platelet count <75×10⁹/L (100,000/mm³);
(d) hemoglobin <90 g/L;
(e) estimated glomerular filtration rate (eGFR)<30 ml/min/1.73 m² calculated according to the Chronic Kidney Disease Epidemiology Collaboration (CKD-EPI) equation; or
(f) aspartate transferase (AST) or alanine transaminase (ALT) >2.5×ULN.
34. The method, use, or mosunetuzumab for use of any one of embodiments 1-33, wherein the individual achieves an evaluable response after completion of the dosing regimen.
35. The method, use, or mosunetuzumab for use of embodiment 34, wherein the evaluable response is remission, a clinical response, a complete renal response (CRR), or a partial renal response.
36. The method, use, or mosunetuzumab for use of embodiment 34 or 35, wherein the evaluable response is remission, and wherein the individual:
(a) maintains a clinical SLE Disease Activity Index=0 for 6 months after administration of the final dose of mosunetuzumab; and
(b) is not administered any additional therapeutic agents for treating SLE, except for an antimalarial agent, for 6 months after administration of the final dose of mosunetuzumab.
37. The method, use, or mosunetuzumab for use of embodiment 36, wherein the antimalarial agent is hydroxychloroquine, chloroquine, or quinacrine.
38. The method, use, or mosunetuzumab for use of embodiment 34 or 35, wherein the evaluable response is a clinical response, and wherein the individual:
(a) maintains a clinical SLE Disease Activity Index (cSLEDAI)=0 for 6 months after administration of the final dose of mosunetuzumab; and
(b) is administered or to be administered a stable dosing of SLE therapy for 6 months after administration of the final dose of mosunetuzumab; and
(c) is administered or to be administered ≤5 mg/day prednisone or equivalent for 6 months after administration of the final dose of mosunetuzumab.
39. The method, use, or mosunetuzumab for use of embodiment 34 or 35, wherein the evaluable response is a complete renal response, wherein the individual has:
(a) urine protein creatinine ratio (UPCR)<0.5 g/g; and
(b) eGFR ≥85% of baseline or ≥60 ml/min/1.73 m² calculated according to the CKD-EPI equation.
40. The method, use, or mosunetuzumab for use of embodiment 34 or 35, wherein the evaluable response is a partial renal response, wherein the individual has:
(a) ≥50% reduction in UPCR from baseline;
(b) UPCR <1 g/g, or UPCR <3 g/g if baseline UPCR was ≥3 g/g; and
(c) eGFR ≥85% of baseline calculated according to the CKD-EPI equation.
41. The method, use, or mosunetuzumab for use of any one of embodiments 1-40, wherein the individual is further administered or to be administered tocilizumab if the individual experiences cytokine release syndrome (CRS).
42. The method, use, or mosunetuzumab for use of any one of embodiments 1-19 and 21-41, further comprising administering to the individual a corticosteroid or a calcineurin inhibitor.
43. The method, use, or mosunetuzumab for use of embodiment 42, wherein:
(a) the corticosteroid comprises hydrocortisone, cortisone acetate, prednisolone, dexamethasone, betamethasone, triamcinolone, beclomethasone, methylprednisolone or prednisone; or
(b) the calcineurin inhibitor comprises ciclosporin, tacrolimus, or vocolosporin.
44. The method, use, or mosunetuzumab for use of any one of embodiments 1-43, wherein the individual has active Class III or IV lupus nephritis (LN), as defined by the 2018 International Society of Nephrology/Renal Pathology Society (ISN/RPS) classification guidelines (Bajema et al., Kidney International. 93(4):389-796, 2018).
45. The method, use, or mosunetuzumab for use of embodiment 44, wherein the Class III or IV LN was determined using a renal biopsy sample.
46. The method, use, or mosunetuzumab for use of embodiment 45, wherein the renal biopsy sample was determined to have ≥3 points on the NIH activity index, excluding any contribution from interstitial activity; and <50% glomerulosclerosis and <50% tubulointerstitial fibrosis.
47. The method, use, or mosunetuzumab for use of any one of embodiments 44-46, wherein the individual has:
(a) UPCR ≥1.15 g/g;
(b) Complement C3 below the lower limit of normal; and/or
(c) active LN that has been treated for at least 6 months with standard nonsteroidal LN therapy prior to administration of mosunetuzumab.
48. The method, use, or mosunetuzumab for use of embodiment 47, wherein the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy.
49. The method, use, or mosunetuzumab for use of embodiment 48, wherein:
(a) the anti-CD19 therapy is blinatumomab;
(b) the anti-CD20 therapy is obinutuzumab, rituximab, ocrelizumab, or ofatumumab;
(c) the kinase inhibitor is baricitinib, tofacitinib, upadacitinib, filgotinib, ibrutinib, or fenebrutinib;
(d) the calcineurin inhibitor is tacrolimus, ciclosporin, or voclosporin; or
(e) the biologic therapy is belimumab, ustekinumab, anifrolumab, secukinumab, or atacicept.
50. The method, use, or mosunetuzumab for use of any one of embodiments 1-43, wherein the individual does not have LN.
51. The method, use, or mosunetuzumab for use of embodiment 50, wherein the individual has autoantibody positivity, hypocomplementemia, and high disease activity despite treatment with advanced therapy.
52. The method, use, or mosunetuzumab for use of embodiment 51, wherein the individual has an anti-double-stranded DNA antibody or anti-Smith antibody above ULN; or a Complement C3 level below the lower limit of normal.
53. The method, use, or mosunetuzumab for use of embodiment 51 or 52, wherein the individual has an Extrarenal SLE Disease Activity Index 2000 (SLEDAI-2K) score ≥8, excluding points for:
(a) proteinuria, pyuria, hematuria, casts, alopecia, fever, and headache;
(b) rash if Cutaneous LE Disease Area and Severity Index (CLASI; Albrecht et al., J Invest Dermatol. 125(5):889-894, 2005) activity score is <6;
(c) mucosal ulcers that do not significantly interfere with oral intake; and
(d) arthritis with <4 joints that are both swollen and tender or with <2 located in the wrist or hand.
54. The method, use, or mosunetuzumab for use of any one of embodiments 51-53, wherein the advanced therapy comprises treatment for at least 6 months with cyclophosphamide, belimumab, ustekinumab, anifrolumab, secukinumab, atacicept, obinutuzumab, rituximab, ocrelizumab, or ofatumumab.
55. The method, use, or mosunetuzumab for use of any one of embodiments 1-54, wherein the individual is administered or to be administered dexamethasone, diphenhydramine, and/or acetaminophen/paracetamol prior to administration of any dose of mosunetuzumab.
56. The method, use, or mosunetuzumab for use of embodiment 55, wherein the individual is administered or to be administered dexamethasone, diphenhydramine, and acetaminophen/paracetamol prior to administration of the C1D1, C1D2, C1D3, and C2D1 of mosunetuzumab.
57. The method, use, or mosunetuzumab for use of embodiment 55 or 56, wherein dexamethasone is administered or to be administered orally at a dose of about 20 mg; diphenhydramine is administered or to be administered orally at a dose of about 25 mg to about 50 mg; and acetaminophen/paracetamol is administered orally at a dose of about 650 mg to about 1000 mg.
58. A method of treating a population of individuals having SLE, comprising subcutaneously administering to each individual of the population mosunetuzumab according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
59. Use of mosunetuzumab for treating systemic lupus erythematosus (SLE) in a population of individuals in need thereof, wherein mosunetuzumab is to be administered subcutaneously to each individual of the population according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
60. Use of mosunetuzumab in the manufacture of a medicament for use in a method of treating systemic lupus erythematosus (SLE) in a population of individuals in need thereof, wherein mosunetuzumab is formulated for subcutaneous administration to each individual of the population according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
61. Mosunetuzumab for use in treating systemic lupus erythematosus (SLE) in a population of individuals in need thereof, wherein mosunetuzumab is to be administered subcutaneously to each individual of the population according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.
62. The method, use, or mosunetuzumab for use of any one of embodiments 58-61, wherein the first dosing cycle is a 21-day dosing cycle.
63. The method, use, or mosunetuzumab for use of any one of embodiments 58-62, wherein the C1D1, C1D2, and C1D3 are administered or to be administered on or about Days 1, 8, and 15, respectively, of the first dosing cycle.
64. The method, use, or mosunetuzumab for use of any one of embodiments 58-63, wherein the second dosing cycle is a 21-day dosing cycle.
65. The method, use, or mosunetuzumab for use of any one of embodiments 58-64, wherein the C2D1 is administered or to be administered on or about Day 1 of the second dosing cycle.
66. The method, use, or mosunetuzumab for use of any one of embodiments 52-65, wherein the method further comprises one or more additional dosing cycles.
67. The method, use, or mosunetuzumab for use of embodiment 66, wherein the method comprises one to seven additional dosing cycles.
68. The method, use, or mosunetuzumab for use of embodiment 66 or 67, wherein the method comprises seven additional dosing cycles.
69. The method, use, or mosunetuzumab for use of any one of embodiments 66-68, wherein each additional dosing cycle is a 21-day dosing cycle.
70. The method, use, or mosunetuzumab for use of any one of embodiments 66-69, wherein one or more of the additional dosing cycles comprises an additional single dose of mosunetuzumab.
71. The method, use, or mosunetuzumab for use of embodiment 70, wherein the additional single dose of mosunetuzumab is administered or to be administered on Day 1 of the respective additional dosing cycle.
72. The method, use, or mosunetuzumab for use of embodiment 70 or 71, wherein the additional single dose of mosunetuzumab is about 45 mg.
73. A method of treating a population of individuals having SLE, comprising subcutaneously administering to each individual of the population mosunetuzumab according to a dosing regimen comprising nine 21-day dosing cycles, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are administered on Days 1, 8, and 15, respectively, of the first dosing cycle; and
(b) the second to ninth dosing cycles each comprises a single dose (C2D1-C9D1) of mosunetuzumab administered on Day 1 of each respective dosing cycle, wherein each of the C2D1-C9D1 is about 45 mg.
74. Use of mosunetuzumab for treating systemic lupus erythematosus (SLE) in a population of individuals in need thereof, wherein mosunetuzumab is to be administered subcutaneously to each individual of the population according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
75. Use of mosunetuzumab in the manufacture of a medicament for use in a method of treating systemic lupus erythematosus (SLE) in a population of individuals in need thereof, wherein mosunetuzumab is formulated for subcutaneous administration to each individual of the population according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
76. Mosunetuzumab for use in treating systemic lupus erythematosus (SLE) in a population of individuals in need thereof, wherein mosunetuzumab is to be administered subcutaneously to each individual of the population according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
77. The method, use, or mosunetuzumab for use of any one of embodiments 58-76, wherein mosunetuzumab is administered or to be administered as a monotherapy.
78. The method, use, or mosunetuzumab for use of any one of embodiments 58-77, wherein prior to administration of any dose of mosunetuzumab, an individual of the population has been previously administered an oral corticosteroid, an antimalarial agent, or an immunosuppressant.
79. The method, use, or mosunetuzumab for use of any one of embodiments 58-78, wherein an individual of the population has been administered an antimalarial agent at a stable dose for at least 4 weeks prior to being administered mosunetuzumab.
80. The method, use, or mosunetuzumab for use of embodiment 79, wherein the antimalarial agent is hydroxychloroquine, chloroquine, or quinacrine.
81. The method, use, or mosunetuzumab for use of any one of embodiments 58-78, wherein an individual of the population has been administered an immunosuppressant at a stable dose for at least 4 weeks prior to being administered mosunetuzumab.
82. The method, use, or mosunetuzumab for use of embodiment 81, wherein the immunosuppressant is azathioprine, mycophenolate mofetil, mycophenolic acid, or methotrexate.
83. The method, use, or mosunetuzumab for use of any one of embodiments 58-82, wherein an individual of the population does not have a lupus-associated neuropsychiatric disease.
84. The method, use, or mosunetuzumab for use of embodiment 83, wherein the lupus-associated neuropsychiatric disease is meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, or mononeuritis multiplex.
85. The method, use, or mosunetuzumab for use of any one of embodiments 58-84, wherein an individual of the population does not have active overlap syndrome with mixed connective tissue disease or systemic sclerosis within a year prior to being administered mosunetuzumab.
86. The method, use, or mosunetuzumab for use of any one of embodiments 58-85, wherein an individual of the population does not have catastrophic or severe antiphospholipid syndrome within a year prior to being administered mosunetuzumab, unless the severe antiphospholipid syndrome has been adequately controlled by administering anticoagulant therapy to the individual at least 2 months prior to being administered mosunetuzumab.
87. The method, use, or mosunetuzumab for use of any one of embodiments 58-86, wherein an individual of the population has not been administered:
(a) at least 12 months prior to being administered mosunetuzumab:
   (i) an anti-CD19 antibody therapy, or
   (ii) an anti-CD20 monoclonal antibody therapy;
(b) at least 30 days prior to being administered mosunetuzumab:
   (i) kinase inhibitors of JAK kinase, Bruton tyrosine kinase, or tyrosine kinase 2, or
   (ii) a calcineurin inhibitor; or
(c) at least 2 months prior to being administered mosunetuzumab:
   (i) cyclophosphamide, or
   (ii) a biologic therapy.
88. The method, use, or mosunetuzumab for use of embodiment 87, wherein:
(a) the anti-CD19 therapy is blinatumomab;
(b) the anti-CD20 therapy is obinutuzumab, rituximab, ocrelizumab, or ofatumumab;
(c) the kinase inhibitors are baricitinib, tofacitinib, upadacitinib, filgotinib, ibrutinib, or fenebrutinib;
(d) the calcineurin inhibitor is tacrolimus, ciclosporin, or voclosporin; or
(e) the biologic therapy is belimumab, ustekinumab, anifrolumab, secukinumab, or atacicept.
89. The method, use, or mosunetuzumab for use of any one of embodiments 58-88, wherein an individual of the population does not have significant lupus-associated renal disease or significant renal impairment.
90 The method, use, or mosunetuzumab for use of any one of embodiments 58-89, wherein an individual of the population does not have any of the following laboratory parameters:

$$( a ) total bilirubin > 1.5 \times ULN ;$$

$$( b ) ANC < 1.5 \times {10}^{9} / L ( < 1500 / μL ) ;$$

$$( c ) platelet count < 75 \times {10}^{9} / L ( 100 , 000 / {mm}^{3} ) ;$$

$$( d ) hemoglobin < 90 g / L ;$$

$$( e ) eGFR < 30 ml / min / 1.73 {m}^{2} calculated according to CKD - EPI equation ;$$

$$or$$

$$( f ) AST or ALT > 2.5 \times ULN .$$

91. The method, use, or mosunetuzumab for use of any one of embodiments 58-90, wherein an individual of the population achieves an evaluable response after completion of the dosing regimen.
92. The method, use, or mosunetuzumab for use of embodiment 91, wherein the evaluable response is remission, a clinical response, a complete renal response (CRR), or a partial renal response.
93. The method, use, or mosunetuzumab for use of embodiment 91 or 92, wherein the evaluable response is remission, and wherein an individual of the population:
(a) maintains a clinical SLE Disease Activity Index=0 for 6 months after administration of the final dose of mosunetuzumab; and
(b) is not administered any additional therapeutic agents for treating SLE, except for an antimalarial agent, for 6 months after administration of the final dose of mosunetuzumab.
94. The method, use, or mosunetuzumab for use of embodiment 93, wherein the antimalarial agent is hydroxychloroquine, chloroquine, or quinacrine.
95. The method, use, or mosunetuzumab for use of embodiment 91 or 92, wherein the evaluable response is a clinical response, and wherein an individual of the population:
(a) maintains a clinical SLE Disease Activity Index (cSLEDAI)=0 for 6 months after administration of the final dose of mosunetuzumab; and
(b) is administered or to be administered a stable dosing of SLE therapy for 6 months after administration of the final dose of mosunetuzumab; and
(c) is administered or to be administered ≤5 mg/day prednisone or equivalent for 6 months after administration of the final dose of mosunetuzumab.
96. The method, use, or mosunetuzumab for use of embodiment 91 or 92, wherein the evaluable response is a complete renal response, wherein an individual of the population has:
(a) urine protein creatinine ratio (UPCR)<0.5 g/g; and
(b) eGFR ≥85% of baseline or ≥60 ml/min/1.73 m² calculated according to the CKD-EPI equation.
97. The method, use, or mosunetuzumab for use of embodiment 91, or 92, wherein the evaluable response is a partial renal response, wherein an individual of the population has:
(a) ≥50% reduction in UPCR from baseline;
(b) UPCR <1 g/g, or UPCR <3 g/g if baseline UPCR was ≥3 g/g; and
(c) eGFR ≥85% of baseline calculated according to the CKD-EPI equation.
98. The method, use, or mosunetuzumab for use of any one of embodiments 58-97, wherein an individual of the population is further administered or to be administered tocilizumab if the individual experiences CRS.
99. The method, use, or mosunetuzumab for use of any one of embodiments 58-76 and 78-98, further comprising administering to an individual of the population a corticosteroid or a calcineurin inhibitor.
100. The method, use, or mosunetuzumab for use of embodiment 99, wherein:
(a) the corticosteroid comprises hydrocortisone, cortisone acetate, prednisolone, dexamethasone, betamethasone, triamcinolone, beclomethasone, methylprednisolone or prednisone; or
(b) the calcineurin inhibitor comprises ciclosporin, tacrolimus, or vocolosporin.
101. The method, use, or mosunetuzumab for use of any one of embodiments 58-100, wherein an individual of the population has active Class III or IV LN, as defined by the 2018 ISN/RPS classification guidelines.
102. The method, use, or mosunetuzumab for use of embodiment 101, wherein the Class III or IV LN was determined using a renal biopsy sample.
103. The method, use, or mosunetuzumab for use of embodiment 102, wherein the renal biopsy sample was determined to have ≥3 points on the NIH activity index, excluding any contribution from interstitial activity; and <50% glomerulosclerosis and <50% tubulointerstitial fibrosis.
104. The method, use, or mosunetuzumab for use of any one of embodiments 101-103, wherein an individual of the population has:

$$( a ) UPCR \geq 1.15 g / g ;$$

(b) Complement C3 below the lower limit of normal; and/or
(c) active LN that has been treated for at least 6 months with standard nonsteroidal LN therapy prior to administration of mosunetuzumab.
105. The method, use, or mosunetuzumab for use of embodiment 104, wherein the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy.
106. The method, use, or mosunetuzumab for use of embodiment 105, wherein:
(a) the anti-CD19 therapy is blinatumomab;
(b) the anti-CD20 therapy is obinutuzumab, rituximab, ocrelizumab, or ofatumumab;
(c) the kinase inhibitor is baricitinib, tofacitinib, upadacitinib, filgotinib, ibrutinib, or fenebrutinib;
(d) the calcineurin inhibitor is tacrolimus, ciclosporin, or voclosporin; or
(e) the biologic therapy is belimumab, ustekinumab, anifrolumab, secukinumab, or atacicept.
107. The method, use, or mosunetuzumab for use of any one of embodiments 58-100, wherein an individual of the population does not have LN.
108. The method, use, or mosunetuzumab for use of embodiment 107, wherein an individual of the population has autoantibody positivity, hypocomplementemia, and high disease activity despite treatment with advanced therapy.
109. The method, use, or mosunetuzumab for use of embodiment 108, wherein an individual of the population has an anti-double-stranded DNA antibody or anti-Smith antibody above ULN; or a Complement C3 level below the lower limit of normal.
110. The method, use, or mosunetuzumab for use of embodiment 108 or 109, wherein an individual of the population has an Extrarenal SLEDAI-2K score ≥8, excluding points for:
(a) proteinuria, pyuria, hematuria, casts, alopecia, fever, and headache;
(b) rash if CLASI activity score is <6;
(c) mucosal ulcers that do not significantly interfere with oral intake; and
(d) arthritis with <4 joints that are both swollen and tender or with <2 located in the wrist or hand.
111. The method, use, or mosunetuzumab for use of any one of embodiments 108-110, wherein the advanced therapy comprises treatment for at least 6 months with cyclophosphamide, belimumab, ustekinumab, anifrolumab, secukinumab, atacicept, obinutuzumab, rituximab, ocrelizumab, or ofatumumab.
112. The method, use, or mosunetuzumab for use of any one of embodiments 58-111, wherein each individual of the population is administered or to be administered dexamethasone, diphenhydramine, and/or acetaminophen/paracetamol prior to administration of any dose of mosunetuzumab.
113. The method, use, or mosunetuzumab for use of embodiment 112, wherein an individual of the population is administered or to be administered dexamethasone, diphenhydramine, and acetaminophen/paracetamol prior to administration of the C1D1, C1D2, C1D3, and C2D1 of mosunetuzumab.
114. The method, use, or mosunetuzumab for use of embodiment 112 or 113, wherein dexamethasone is administered or to be administered orally at a dose of about 20 mg; diphenhydramine is administered or to be administered orally at a dose of about 25 mg to about 50 mg; and acetaminophen/paracetamol is administered or to be administered orally at a dose of about 650 mg to about 1000 mg.
115. The method, use, or mosunetuzumab for use of any one of embodiments 1-114, wherein mosunetuzumab is administered or to be administered to the individual or individuals of the population as a pharmaceutical composition comprising about 1 mg/ml, 10 mg/ml, or 45 mg/ml mosunetuzumab, about 10 mM L-histidine, about 240 mM sucrose, about 0.06% (w/v) PS20, and about 10 mM methionine, wherein the pharmaceutical composition has a pH of about 5.8.
116. The method, use, or mosunetuzumab for use of embodiment 115, further comprising histidine acetate.
117. The method, use, or mosunetuzumab for use of any one of embodiments 1-57, 115, and 116, wherein the individual exhibits a decrease in titer of antinuclear antibody (ANA), a decrease in titer of anti-double stranded DNA (dsDNA) antibody (IgG) titer, an increase in complement C3 levels, or an increase in complement C4 levels after completion of the dosing regimen.
118. The method, use, or mosunetuzumab for use of any one of embodiments 58-117, wherein an individual of the population exhibits a decrease in titer of antinuclear antibody (ANA), a decrease in titer of anti-double stranded DNA (dsDNA) antibody (IgG) titer, an increase in complement C3 levels, or an increase in complement C4 levels.
119. The method, use, or mosunetuzumab for use of any one of embodiments 1-106, wherein administering one or more doses of mosunetuzumab to the individual or individuals induces a B cell depletion in the individual or individuals.
120. The method, use, or mosunetuzumab for use of embodiment 119, wherein the B cell depletion is a deep-tissue B cell depletion and/or a peripheral B cell depletion.
121. The method, use, or mosunetuzumab for use of embodiment 120, wherein the deep-tissue B cell depletion is determined by tissue biopsy.
122. The method, use, or mosunetuzumab for use of embodiment 121, wherein the deep-tissue B cell depletion is determined by kidney biopsy.
123. The method, use, or mosunetuzumab for use of embodiment 120, wherein the peripheral B cell depletion is determined by blood flow cytometry.
124. The method, use, or mosunetuzumab for use of any one of embodiments 119-123, wherein the amount of B cell depletion is determined relative to a reference level of B cells.
125. The method, use, or mosunetuzumab for use of embodiment 124, wherein the reference level of B cells is determined at a time prior to administration of any dose of mosunetuzumab.
126. The method, use, or mosunetuzumab for use of any one of embodiments 119-125, further comprising discontinuing administering a background immunotherapy to the individual.
127. The method, use, or mosunetuzumab for use of embodiment 126, wherein the background immunotherapy comprises at least one selected from the group consisting of mycophenolate mofetil, azathioprine, and methotrexate.
128. A method of inducing B cell depletion in an individual, comprising subcutaneously administering to the individual mosunetuzumab according to a dosing regimen comprising nine 21-day dosing cycles, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are administered on Days 1, 8, and 15, respectively, of the first dosing cycle; and
(b) the second to ninth dosing cycles each comprises a single dose (C2D1-C9D1) of mosunetuzumab administered on Day 1 of each respective dosing cycle, wherein each of the C2D1-C9D1 is about 45 mg.
129. Use of mosunetuzumab for inducing B cell depletion in an individual, wherein mosunetuzumab is to be administered subcutaneously to the individual according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
130. Use of mosunetuzumab in the manufacture of a medicament for use in a method of inducing B cell depletion in an individual, wherein mosunetuzumab is formulated for subcutaneous administration to the individual according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
131. Mosunetuzumab for use in inducing B cell depletion in an individual, wherein mosunetuzumab is to be administered subcutaneously to the individual according to a dosing regimen comprising at least a first dosing cycle and a second dosing cycle, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are to be administered on Days 1, 8, and 15, respectively, of the first dosing cycle; and
(b) the second to ninth dosing cycles each comprises a single dose (C2D1-C9D1) of mosunetuzumab to be administered on Day 1 of each respective dosing cycle, wherein each of the C2D1-C9D1 is about 45 mg.
132. The method, use, or mosunetuzumab for use of any one of embodiments 128-131, wherein the B cell depletion is a deep-tissue B cell depletion and/or a peripheral B cell depletion.
133. The method, use, or mosunetuzumab for use of embodiment 132, wherein the deep-tissue B cell depletion is determined by tissue biopsy.
134. The method, use, or mosunetuzumab for use of embodiment 133, wherein the deep-tissue B cell depletion is determined by kidney biopsy.
135. The method, use, or mosunetuzumab for use of embodiment 132, wherein the peripheral B cell depletion is determined by blood flow cytometry.
136. The method, use, or mosunetuzumab for use of any one of embodiments 128-135, wherein the amount of B cell depletion is determined relative to a reference level of B cells.
137. The method of embodiment 136, wherein the reference level of B cells is determined at a time prior to administration of any dose of mosunetuzumab.
138. The method, use, or mosunetuzumab for use of any one of embodiments 128-137, wherein the individual has SLE.
139. The method, use, or mosunetuzumab for use of any one of embodiments 132-138, further comprising discontinuing administering a background immunotherapy to the individual.
140. The method, use, or mosunetuzumab for use of embodiment 139, wherein the background immunotherapy comprises at least one selected from the group consisting of mycophenolate mofetil, azathioprine, and methotrexate.
141. A method of treating an individual having systemic lupus erythematosus (SLE), comprising subcutaneously administering to the individual mosunetuzumab according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg, and wherein the method further comprises discontinuing administering a background immunotherapy to the individual.
142. Use of mosunetuzumab for treating an individual having systemic lupus erythematosus (SLE), wherein mosunetuzumab is to be administered subcutaneously to the individual according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg, and
wherein administration of a background immunotherapy to the individual is to be discontinued.
143. Use of mosunetuzumab in the manufacture of a medicament for use in a method of treating an individual having systemic lupus erythematosus (SLE), wherein mosunetuzumab is formulated for subcutaneous administration to the individual according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg, and wherein administration of a background immunotherapy to the individual is to be discontinued.
144. Mosunetuzumab for use in treating an individual having systemic lupus erythematosus (SLE), wherein mosunetuzumab is to be subcutaneously administered to the individual according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg, and
wherein administration of a background immunotherapy to the individual is to be discontinued.
145. The method, use, or mosunetuzumab for use of any one of embodiments 141-144, wherein the individual has B cell depletion after dosing the individual with mosunetuzumab before reducing or removing background immunotherapy to the individual.
146. The method, use, or mosunetuzumab for use of embodiment 145, wherein the B cell depletion is a deep-tissue B cell depletion and/or a peripheral B cell depletion.
147. The method, use, or mosunetuzumab for use of embodiment 146, wherein the deep-tissue B cell depletion is determined by tissue biopsy.
148. The method, use, or mosunetuzumab for use of embodiment 147, wherein the deep-tissue B cell depletion is determined by kidney biopsy.
149. The method, use, or mosunetuzumab for use of embodiment 146, wherein the peripheral B cell depletion is determined by blood flow cytometry.
150. The method, use, or mosunetuzumab for use of any one of embodiments 141-149, wherein the amount of B cell depletion is determined relative to a reference level of B cells.
151. The method, use, or mosunetuzumab for use of embodiment 150, wherein the reference level of B cells is determined at a time prior to administration of any dose of mosunetuzumab.
152. The method, use, or mosunetuzumab for use of any one of embodiments 141-151, wherein the background immunotherapy comprises at least one selected from the group consisting of mycophenolate mofetil, azathioprine, and methotrexate.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating an individual having systemic lupus erythematosus (SLE), comprising subcutaneously administering to the individual mosunetuzumab according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.

2. The method of claim 1, wherein;
(a) the first dosing cycle is a 21-day dosing cycle;
(b) the second dosing cycle is a 21-day dosing cycle; and/or
(c) the method further comprises one or more additional dosing cycles.

3. The method of claim 2, wherein;
(a) the C1D1, C1D2, and C1D3 are administered on or about Days 1, 8, and 15, respectively, of the first dosing cycle;
(b) the C2D1 is administered on or about Day 1 of the second dosing cycle;
(c) the method comprises seven additional dosing cycles;
(d) each additional dosing cycle is a 21-day dosing cycle; and/or
(e) one or more of the additional dosing cycles comprises an additional single dose of mosunetuzumab.

4-10. (canceled)

11. The method of claim 3, wherein the additional single dose of mosunetuzumab is administered on Day 1 of the respective additional dosing cycle; and/or the additional single dose of mosunetuzumab is about 45 mg.

12-13. (canceled)

14. The method of claim 1, wherein:
(a) mosunetuzumab is administered as a monotherapy;
(b) prior to administration of any dose of mosunetuzumab, the individual has been previously administered an oral corticosteroid, an antimalarial agent, or an immunosuppressant;
(c) the individual does not have a lupus-associated neuropsychiatric disease;
(d) the individual does not have active overlap syndrome with mixed connective tissue disease or systemic sclerosis within a year prior to being administered mosunetuzumab;
(e) the individual does not have catastrophic or severe antiphospholipid syndrome within a year prior to being administered mosunetuzumab, unless the severe antiphospholipid syndrome has been adequately controlled by administering anticoagulant therapy to the individual at least 2 months prior to being administered mosunetuzumab;
(f) the individual has not been administered:
(i) at least 12 months prior to being administered mosunetuzumab:
(1) an anti-CD19 antibody therapy, or
(2) an anti-CD20 monoclonal antibody therapy;
(ii) at least 30 days prior to being administered mosunetuzumab:
(1) kinase inhibitors of Janus Kinase (JAK) kinase, Bruton tyrosine kinase, or tyrosine kinase 2, or
(2) a calcineurin inhibitor; or
(iii) at least 2 months prior to being administered mosunetuzumab:
(1) cyclophosphamide, or
(2) a biologic therapy;
(g) the individual does not have significant lupus-associated renal disease or significant renal impairment; and/or
(h) the individual does not have any of the following laboratory parameters:
(i) total bilirubin >1.5×upper limit of normal (ULN);
(ii) absolute neutrophil count (ANC)<1.5×109/L (<1500/μL);
(iii) platelet count <75×109/L (100,000/mm3);
(iv) hemoglobin <90 g/L;
(v) estimated glomerular filtration rate (eGFR)<30 ml/min/1.73 m2 calculated according to the Chronic Kidney Disease Epidemiology Collaboration (CKD-EPI) equation; or
(vi) aspartate transferase (AST) or alanine transaminase (ALT) >2.5×ULN.

15. (canceled)

16. The method of claim 14, wherein:
(a) the individual has been administered an antimalarial agent at a stable dose for at least 4 weeks prior to being administered mosunetuzumab; and/or the antimalarial agent is hydroxychloroquine, chloroquine, or quinacrine;
(b) the individual has been administered an immunosuppressant at a stable dose for at least 4 weeks prior to being administered mosunetuzumab; and/or the immunosuppressant is azathioprine, mycophenolate mofetil, mycophenolic acid, or methotrexate; and/or
(c) the lupus-associated neuropsychiatric disease is meningitis, retinitis, cerebral vasculitis, myelopathy, demyelination syndromes, acute confusional state, psychosis, acute stroke or stroke syndrome, cranial neuropathy, status epilepticus or seizures, cerebellar ataxia, or mononeuritis multiplex;
(d) the anti-CD19 therapy is blinatumomab;
(e) the anti-CD20 therapy is obinutuzumab, rituximab, ocrelizumab, or ofatumumab;
(f) the kinase inhibitors are baricitinib, tofacitinib, upadacitinib, filgotinib, ibrutinib, or fenebrutinib;
(q) the calcineurin inhibitor is tacrolimus, ciclosporin, or voclosporin; or
(h) the biologic therapy is belimumab, ustekinumab, anifrolumab, secukinumab, or atacicept.

17-27. (canceled)

28. The method of claim 1, wherein the individual achieves an evaluable response after completion of the dosing regimen.

29. The method of claim 28, wherein the evaluable response is remission, a clinical response, a complete renal response (CRR), or a partial renal response.

30. The method of claim 29, wherein the evaluable response is:
(a) remission, and wherein the individual:
(i) maintains a clinical SLE Disease Activity Index=0 for 6 months after administration of the final dose of mosunetuzumab; and
(ii) is not administered any additional therapeutic agents for treating SLE, except for an antimalarial agent, for 6 months after administration of the final dose of mosunetuzumab;
(b) a clinical response, and wherein the individual;
(i) maintains a clinical SLE Disease Activity Index (cSLEDAI)=0 for 6 months after administration of the final dose of mosunetuzumab; and
(ii) is administered a stable dosing of SLE therapy for 6 months after administration of the final dose of mosunetuzumab; and
(iii) is administered ≤5 mg/day prednisone or equivalent for 6 months after administration of the final dose of mosunetuzumab
(c) a complete renal response, wherein the individual has:
(i) urine protein creatinine ratio (UPCR)<0.5 q/q; and
(ii) eGFR ≥85% of baseline or ≥60 ml/min/1.73 m2 calculated according to the CKD-EPI equation; or
(d) a partial renal response, wherein the individual has:
(i) ≥50% reduction in UPCR from baseline;
(ii) UPCR <1 q/q, or UPCR <3 q/q if baseline UPCR was ≥3 q/q; and
(iii) eGFR ≥85% of baseline calculated according to the CKD-EPI equation.

31. The method of claim 30, wherein the antimalarial agent is hydroxychloroquine, chloroquine, or quinacrine.

32-34. (canceled)

35. The method of claim 1, wherein the individual is further administered tocilizumab if the individual experiences cytokine release syndrome (CRS).

36. The method of claim 1, further comprising administering to the individual a corticosteroid or a calcineurin inhibitor.

37. The method of claim 36, wherein:
(a) the corticosteroid comprises hydrocortisone, cortisone acetate, prednisolone, dexamethasone, betamethasone, triamcinolone, beclomethasone, methylprednisolone or prednisone; or
(b) the calcineurin inhibitor comprises ciclosporin, tacrolimus, or vocolosporin.

38. The method of claim 1, wherein the individual has active Class III or IV lupus nephritis (LN), as defined by the 2018 International Society of Nephrology/Renal Pathology Society (ISN/RPS) classification guidelines (Bajema et al., Kidney International. 93(4):389-796, 2018).

39. The method of claim 38, wherein;
(a) the Class III or IV LN was determined using a renal biopsy sample; and/or
(b) the individual has:
(i) UPCR ≥1.15 q/a;
(ii) Complement C3 below the lower limit of normal; and/or
(iii) active LN that has been treated for at least 6 months with standard nonsteroidal LN therapy prior to administration of mosunetuzumab.

40. The method of claim 39, wherein;
(a) the renal biopsy sample was determined to have ≥3 points on the NIH activity index, excluding any contribution from interstitial activity; and <50% glomerulosclerosis and <50% tubulointerstitial fibrosis; and/or
(b) the standard nonsteroidal LN therapy comprises an anti-CD19 antibody therapy; an anti-CD20 monoclonal antibody therapy; a kinase inhibitor of JAK kinase, Bruton tyrosine kinase, or tyrosine kinase 2; a calcineurin inhibitor; cyclophosphamide; or a biologic therapy.

41-42. (canceled)

43. The method of claim 40, wherein:
(a) the anti-CD19 therapy is blinatumomab;
(b) the anti-CD20 therapy is obinutuzumab, rituximab, ocrelizumab, or ofatumumab;
(c) the kinase inhibitor is baricitinib, tofacitinib, upadacitinib, filgotinib, ibrutinib, or fenebrutinib;
(d) the calcineurin inhibitor is tacrolimus, ciclosporin, or voclosporin; or
(e) the biologic therapy is belimumab, ustekinumab, anifrolumab, secukinumab, or atacicept.

44. The method of claim 1, wherein the individual does not have LN.

45. The method of claim 44, wherein the individual has autoantibody positivity, hypocomplementemia, and high disease activity despite treatment with advanced therapy.

46. The method of claim 45, wherein;
(a) the individual has an anti-double-stranded DNA antibody or anti-Smith antibody above ULN; or a Complement C3 level below the lower limit of normal;
(b) the individual has an Extrarenal SLE Disease Activity Index 2000 (SLEDAI-2K) score ≥8, excluding points for:
(i) proteinuria, pyuria, hematuria, casts, alopecia, fever, and headache;
(ii) rash if Cutaneous LE Disease Area and Severity Index (CLASI; Albrecht et al., J Invest Dermatol. 125(5):889-894, 2005) activity score is <6;
(iii) mucosal ulcers that do not significantly interfere with oral intake; and
(iv) arthritis with <4 joints that are both swollen and tender or with <2 located in the wrist or hand; and/or
(c) the advanced therapy comprises treatment for at least 6 months with cyclophosphamide, belimumab, ustekinumab, anifrolumab, secukinumab, atacicept, obinutuzumab, rituximab, ocrelizumab, or ofatumumab.

47-48. (canceled)

49. The method of claim 1, wherein the individual is administered dexamethasone, diphenhydramine, and/or acetaminophen/paracetamol prior to administration of any dose of mosunetuzumab.

50. The method of claim 49, wherein;
(a) the individual is administered dexamethasone, diphenhydramine, and acetaminophen/paracetamol prior to administration of the C1D1, C1D2, C1D3, and C2D1 of mosunetuzumab; and/or
(b) dexamethasone is administered orally at a dose of about 20 ma; diphenhydramine is administered orally at a dose of about 25 ma to about 50 ma; and acetaminophen/paracetamol is administered orally at a dose of about 650 ma to about 1000 ma.

51. (canceled)

52. A method of treating a population of individuals having SLE, comprising subcutaneously administering to each individual of the population mosunetuzumab according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg.

53-63. (canceled)

64. A method of treating a population of individuals having SLE, comprising subcutaneously administering to each individual of the population mosunetuzumab according to a dosing regimen comprising nine 21-day dosing cycles, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are administered on Days 1, 8, and 15, respectively, of the first dosing cycle; and
(b) the second to ninth dosing cycles each comprises a single dose (C2D1-C9D1) of mosunetuzumab administered on Day 1 of each respective dosing cycle, wherein each of the C2D1-C9D1 is about 45 mg.

65-78. (canceled)

79. The method of claim 52, wherein an individual of the population achieves an evaluable response after completion of the dosing regimen.

80. The method of claim 79, wherein the evaluable response is remission, a clinical response, a complete renal response (CRR), or a partial renal response.

81. The method of claim 80, wherein:
(a) the evaluable response is remission, and wherein an individual of the population:
(i) maintains a clinical SLE Disease Activity Index=0 for 6 months after administration of the final dose of mosunetuzumab; and
(ii) is not administered any additional therapeutic agents for treating SLE, except for an antimalarial agent, for 6 months after administration of the final dose of mosunetuzumab;
(b) the evaluable response is a clinical response, and wherein an individual of the population:
(i) maintains a clinical SLE Disease Activity Index (cSLEDAI)=0 for 6 months after administration of the final dose of mosunetuzumab; and
(ii) is administered a stable dosing of SLE therapy for 6 months after administration of the final dose of mosunetuzumab; and
(iii) is administered ≤5 mg/day prednisone or equivalent for 6 months after administration of the final dose of mosunetuzumab;
(c) the evaluable response is a complete renal response, wherein an individual of the population has:
(i) urine protein creatinine ratio (UPCR)<0.5 q/q; and
(ii) eGFR ≥85% of baseline or ≥60 ml/min/1.73 m2 calculated according to the CKD-EPI equation; or
(d) the evaluable response is a partial renal response, wherein an individual of the population has:
(i) ≥50% reduction in UPCR from baseline;
(ii) UPCR <1 q/q, or UPCR <3 q/q if baseline UPCR was ≥3 q/q; and
(iii) eGFR ≥85% of baseline calculated according to the CKD-EPI equation.

82. The method of claim 81, wherein the antimalarial agent is hydroxychloroquine, chloroquine, or quinacrine.

83-102. (canceled)

103. The method of claim 1, wherein mosunetuzumab is administered to the individual or the individuals of the population as a pharmaceutical composition comprising about 1 mg/ml, 10 mg/ml, or 45 mg/ml mosunetuzumab, about 10 mM L-histidine, about 240 mM sucrose, about 0.06% (w/v) PS20, and about 10 mM methionine, wherein the pharmaceutical composition has a pH of about 5.8.

104. (canceled)

105. The method of claim 1, wherein the individual exhibits a decrease in titer of antinuclear antibody (ANA), a decrease in titer of anti-double stranded DNA (dsDNA) antibody (IgG) titer, an increase in complement C3 levels, or an increase in complement C4 levels after completion of the dosing regimen.

106-115. (canceled)

116. A method of inducing B cell depletion in an individual, comprising subcutaneously administering to the individual mosunetuzumab according to a dosing regimen comprising nine 21-day dosing cycles, wherein:
(a) the first dosing cycle comprises a first dose (C1D1), a second dose (C1D2), and a third dose (C1D3), wherein the C1D1 of mosunetuzumab is about 1.6 mg, the C1D2 of mosunetuzumab is about 5 mg, and the C1D3 of mosunetuzumab is about 45 mg, and wherein the C1D1, the C1D2, and the C1D3 are administered on Days 1, 8, and 15, respectively, of the first dosing cycle; and
(b) the second to ninth dosing cycles each comprises a single dose (C2D1-C9D1) of mosunetuzumab administered on Day 1 of each respective dosing cycle, wherein each of the C2D1-C9D1 is about 45 mg.

117. The method of claim 116, wherein;
(a) the B cell depletion is a deep-tissue B cell depletion and/or a peripheral B cell depletion;
(b) the amount of B cell depletion is determined relative to a reference level of B cells;
(c) the individual has SLE; and/or
(d) the method further comprises discontinuing administering a background immunotherapy to the individual.

118. The method of claim 117, wherein;
(a) the deep-tissue B cell depletion is determined by tissue biopsy; or the peripheral B cell depletion is determined by blood flow cytometry;
(b) the reference level of B cells is determined at a time prior to administration of any dose of mosunetuzumab; and/or
(c) the background immunotherapy comprises at least one selected from the group consisting of mycophenolate mofetil, azathioprine, and methotrexate.

119. The method of claim 118, wherein the deep-tissue B cell depletion is determined by kidney biopsy.

120-125. (canceled)

126. A method of treating an individual having systemic lupus erythematosus (SLE), comprising subcutaneously administering to the individual mosunetuzumab according to a dosing regimen comprising at least a first dosing cycle and a second dosing cycle, wherein:
(a) the first dosing cycle comprises a first dose (C1D1) of mosunetuzumab, a second dose (C1D2) of the mosunetuzumab, and a third dose (C1D3) of mosunetuzumab, wherein the C1D1 of mosunetuzumab is about 1.6 mg, the C1D2 of mosunetuzumab is about 5 mg, and the C1D3 of mosunetuzumab is about 45 mg; and
(b) the second dosing cycle comprises a single dose (C2D1) of mosunetuzumab, wherein the C2D1 of mosunetuzumab is about 45 mg, and
wherein the method further comprises discontinuing administering a background immunotherapy to the individual.

127-134. (canceled)
